SEQUENCE LISTING (171 sequences)

SEQ ID NO: 1 - protein, 1068 aa, Prochlorococcus marinus
MESPSSWKTSWLAAIAPDEPHKFDRRLEWDELSEENFFAALNSEPASLEEDDPCFEEALQDALEALKAAWDLPLLPVDNNLNRPFVDVWWPIRCHSAESLRQSFVSDSAGLADEIFDQLADSLLDRLCALGDQVLWEAFNKERTPGTMLLAHLGAAGDGSGPPVREHYERFIQSHRRNGLAPLLKEFPVLGRLIGTVLSLWFQGSVEMLQRICADRTVLQQCFAIPCGHHLKTVKQGLSDPHRGGRAVAVLEFADPNSTANSSMHVVYKPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAEVKQMQHLDIPFFTHLIDADALQLGGLEQELPGFIQTSGLAAAYERLRNLDTDEIAFQLRLIRGAVEARELHTTPESSPTLPPPATPEALMSSSAETSLEAAKRIAHRLLELAIRDSQGQVEWLGMDLGADGESFSFGPVGLSLYGGSIGIAHLLQRLQAQQVSLMDADAIQTAILQPLVGLVDQPSDDGRRRWWRDQPLGLSGCGGTLLALTLQGEQAMANSLLAAALPRFIEADQQLDLIGGCAGLIGSLVQLGTESALQLALRAGDHLIAQQNEEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCCGSLGLMVLLEMLSAGPWPIDNQLRSHCQDVAFQYRLQALQRCSAEPIKLRCFGTKEGLLVLPGFFTGLSGMGLALLEDDPSRAVVSQLISAGLWPTE

SEQ ID NO: 2 - protein, 1068 aa, Prochlorococcus marinus
MTSPTSWKTSWLAAIAPDEPHKFDRRLEWDELSEENFFAALNSAPTSLEEDDPCFDEALQDALEALKAAWDLPLLPVDNTVNRPFVDLWWPIRCHSAESLRQIFVSDSAGLADEIFDQLADSLLDRLCALGDQVLWEAFNKERTPGTMLLAHLGAAGDGSGPPVREHYERFIQSHRRNGLAPLLKEFPVLGRLIDTVLSLWFQGSVEMLQRICADRTVLQQGFAIPCGHHLKTVKQGLSDPHRGGRAVAVLEFADPNSTANSSMHVVYKPKDMAVDAAYQATLADLNAHSDLSPLRTLAIHNGDGYGYMEHVVHHFCANDKELTNFYFNAGRLTALLHLLGCTDCHHENLIACGDQLLLIDTETLLEADLPDHISDASSTTAQPEPSTLQRQFQRSVLRSGLLPQWMFLGESKQAIDISALGISPPNKPERIAAGWLGFNSDGMMPGRVSQPVEIPTSLPVGIADVNPFDRFLEDFCDGFSMQSKALIKLRNRWLDLNGILAHFAGLPRRIVLRATRVYFNIQRQQLEPTALRSSLAQALKLEQLARSFLLAESKPLHWPIFAAEVKQMQQLDIPFFTHLIDADALQLGGLEQELPGFIQTSGLAAAYERLRNLDSNEIAFQLRLIHGAVEARELHTTPESSPTLHPPATPEALMSASAETSLKAAKRIAHRLLELAIRDSQGQVEWLGMDLGADGESFAFGPVGLSLYGGSIGISHLLQRLQAQQVPLMDADAIQTAILQPLVGLVAQPSDDGRRRWWRDQPLGLSGCGGTLLALALQGEQAMANSLLAAALPRFIEADQQLDLIGGCAGLIGSLVQLGTESALQWALRAGDHLIAQQNEEGAWSSSSSQPALLGFSHGVAGYAAALAHLHAFSGDERYRIAAAAALAYERARFNKDAGNWPDYRSISRDSDSDEPSFMASWCHGAPGIALGRACLWGTALWDEECTKEIGIGLQTTAAVSAVSTDHLCCGSLGLMVLLEMLSKGPWPIDNQLRSHCQDVASQYRLQALQRCSAEPIKLRCFGTKEGLLVLPGFFTGLSGMGLALLEDDPSRAVVSQLISAGLWPTE

SEQ ID NO: 3 - protein, 1058 aa, Synechococcus sp.
MRENWKQQWLDIVAPDEPHKLSRRLDWDELTEEEFREHLSTSPTKANRFEGHWQNCLNECRLAIQKAWEQPLLPISPDNQQRPFEDLWRPICNEAREILKKRLSRLDGVGEGIFEQLTNSLLNRLCSIGEQVLWEKFNSERSPGVMLLAHLGSNGDGSGPPVREGYEQFIRNHRQDGLTQLLGEFPELGRLIGTIVLLWLEGSQEMLERIDQDRQTLETLFGIPKNAPLQTIHQGLSDPHRGGRAVAILGFAGPGEEQYKLVYKPKDMAVDAAYQDLLKDINRSSSDKPLKILKIHNGEGYGYMEYVSHKPARDDKELGFFYKNAGRLTAVLHLLGCTDCHHENLIACGDQLLLIDTETLLEAELPNHINNDTSEAPELSALQKRCQRSILRSGLMPQWMFLGSEKIAIDISALGISPPKQETQKTSGWLGLNSDGMMPGVIERNADLPTSLPIGIGSPNPFSLHLDTFCEGFREQNQTLIEQKDQWLKEGTFLNRFKGLRRRIVLRATRVYFAIQRQQLEPAALRSPLIQAIKLEQLSRSFLLAEQKPLHWPVFAAELEQMQQLDIPFFTHQIDSDALDLNQAQTQLPGYIETSGLSAAKERLEQLDTSEVEFQIKLIRGTCNAQKIRRGASVETSVHSAQENQYTTQTKSAADHQVAATNVMNCLADLAIEDNSGQMEWLGMDLGADGESFSFGPVGFSLYGGSSGVAILDHTLQRLNGTNSQLSAIQSGVLQSIQQLAEKASEDQRLRWWRDQSLGLSGCGGMLLTLQCLGQTSWAQSLLKSGRQRFLEADRQLDLIGGCTGLIGPLLHLKSPEALDLAIHAGKHLLEQQQEDGSWPGRAKNTPGLLGFSHGTAGHAAALAKLHQATHDNRFLEGAKAALHHERAHFDSKEENWPDFRDITQSGSFMTSWCHGAPGIGIARACLWQTELWDEQCRDEIHAAIRTTSKSIGQLRSDHLCCGKAGLAIILEMLCDGPWGVEAHVADLARERTIQIHTSLMNRTSSKMPDLICFAAKETSLVLPGFFTGLSGIGMALLRDDQSKQTTRRLLTAGLWTE

SEQ ID NO: 4 - protein, 297 aa, Prochlorococcus marinus
KPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAE

SEQ ID NO: 5 - protein, 131 aa, Prochlorococcus marinus
EEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCC

SEQ ID NO: 6 - protein, 285 aa, Prochlorococcus marinus
ATLADLNAHSDLSPLRTLAIHNGDGYGYMEHVVHHFCANDKELTNFYFNAGRLTALLHLLGCTDCHHENLIACGDQLLLIDTETLLEADLPDHISDASSTTAQPEPSTLQRQFQRSVLRSGLLPQWMFLGESKQAIDISALGISPPNKPERIAAGWLGFNSDGMMPGRVSQPVEIPTSLPVGIADVNPFDRFLEDFCDGFSMQSKALIKLRNRWLDLNGILAHFAGLPRRIVLRATRVYFNIQRQQLEPTALRSSLAQALKLEQLARSFLLAESKPLHWPIFAAE

SEQ ID NO: 7 - protein, 131 aa, Prochlorococcus marinus
EEGAWSSSSSQPALLGFSHGVAGYAAALAHLHAFSGDERYRIAAAAALAYERARFNKDAGNWPDYRSISRDSDSDEPSFMASWCHGAPGIALGRACLWGTALWDEECTKEIGIGLQTTAAVSAVSTDHLCC

SEQ ID NO: 8 - protein, 295 aa, Synechococcus sp.
KPKDMAVDAAYQDLLKDINRSSSDKPLKILKIHNGEGYGYMEYVSHKPARDDKELGFFYKNAGRLTAVLHLLGCTDCHHENLIACGDQLLLIDTETLLEAELPNHINNDTSEAPELSALQKRCQRSILRSGLMPQWMFLGSEKIAIDISALGISPPKQETQKTSGWLGLNSDGMMPGVIERNADLPTSLPIGIGSPNPFSLHLDTFCEGFREQNQTLIEQKDQWLKEGTFLNRFKGLRRRIVLRATRVYFAIQRQQLEPAALRSPLIQAIKLEQLSRSFLLAEQKPLHWPVFAAE

SEQ ID NO: 9 - protein, 109 aa, Synechococcus sp.
HGTAGHAAALAKLHQATHDNRFLEGAKAALHHERAHFDSKEENWPDFRDITQSGSFMTSWCHGAPGIGIARACLWQTELWDEQCRDEIHAAIRTTSKSIGQLRSDHLCC

SEQ ID NO: 10 - protein, 74 aa, Artificial Sequence
  Leader
MKKRLNNQSMSEEQLKAFIAKVQADTSLQEQLKAEGADVVAIAKAAGFSITTEDLEKEHRQTLSDDDLEGVAGG

SEQ ID NO: 11 - protein, 65 aa, Artificial Sequence
  Leader
MSEEQLKAFIAKVQADTSLQEQLKAEGADVVAIAKAAGFSITTEDLEKEHRQTLSDDDLEGVAGG

SEQ ID NO: 12 - protein, 67 aa, Artificial Sequence
  Leader
MSEEQLKAFIAKVQADPSLQEQLKAEGADVVSIAKAAGFSITTEDLNSHITTKLNLSEEELEGVAGA

SEQ ID NO: 13 - protein, 64 aa, Artificial Sequence
  Leader
MSEEQLKGFLSKVQSDASLQEQLKVEGADVVAIAKAAGFSITTEDLNSHRQNLSEDELEGVAGG

SEQ ID NO: 14 - protein, 64 aa, Artificial Sequence
  Leader
MSEEQLKAFLTKVQADTSLQEQLKAEGADPVAIAKAAGFAITTEDLNSHRQNLSDDELEGVAGG

SEQ ID NO: 15 - protein, 64 aa, Artificial Sequence
  Leader
MSEEQLKAFIAKVQADTSLQEQLKVEGADVVAIAKAAGFSITTEDLNTHRQNLSDDELEGLHGA

SEQ ID NO: 16 - protein, 66 aa, Artificial Sequence
  Leader
MSEEQLKAFIAKVQADTSLQEQLKVEGADVVAIAKAAGFSITTDDFERNTHRQTLSDDELEGVAGG

SEQ ID NO: 17 - protein, 76 aa, Artificial Sequence
  Leader
MKHRQLNLMSEEQLKAFIAKVQADTSLQEQLKVEGADVVAIAKASGFAITTEDLKAHQANSQKNLSDAELEGVAGG

SEQ ID NO: 18 - protein, 68 aa, Artificial Sequence
  Leader
MSEEQLKAFIAKVQADTSLQEQLKVEGADVVAIAKASGFAITTEDLKAHQANSQKNLSDAELEGVAGG

SEQ ID NO: 19 - protein, 65 aa, Artificial Sequence
  Leader
MSEEQLKAFIAKVQADSSLQEQLKAEGADVVAIAKAAGFSITTEDWDQRPVRTLSDEELEGAAGG

SEQ ID NO: 20 - protein, 64 aa, Artificial Sequence
  Leader
MSEEQLKAFIAKVQADPSLQEQLKAEGADTVAIAKAAGFSITTEDLKEHRQTLSDDELESVAGG

SEQ ID NO: 21 - protein, 62 aa, Artificial Sequence
  Leader
MSEEQLKAFLEKVKADTSLKEKLKAAKSPEDVVGIAKEHGHEFTADKISQLSEEELEGVAGG

SEQ ID NO: 22 - protein, 65 aa, Artificial Sequence
  Leader
MSEEQLKAFLAKVQADASLQEQLKAEGADVVAIAKAAGFSITTEDLNSHRQIEMTDDELEGVAGG

SEQ ID NO: 23 - protein, 65 aa, Artificial Sequence
  Leader
MSEEQLKAFIAKVQGDTSLQEQLKAEGADVVAIAKAAGFAITEAEVKAYQTRNLSDEELDEVAGG

SEQ ID NO: 24 - protein, 64 aa, Artificial Sequence
  Leader
MSEEQLKAFIAKVQADTSLQEQLKAEGADVVAIAKAAGFAISTEDLNNHRQNLSDDELEGVAGG

SEQ ID NO: 25 - protein, 64 aa, Artificial Sequence
  Leader
MSEEQLKAFIAKVQADTSLQEQLKAEGADVVAIAKAAGFSIATEDLKTHRQTLSDDDLEGVAGG

SEQ ID NO: 26 - protein, 64 aa, Artificial Sequence
  Leader
MSEEQLKAFLTKVQADTSLQEQLKIEGADVVAIAKAAGFSITTEDLNSHRQNLSDDELEGVAGG

SEQ ID NO: 27 - protein, 64 aa, Artificial Sequence
  Leader
MSEEQLKAFIAKVQADPSLQEQLKAEGADVVAIAKAAGFTITTEDLKTARQTLSDDDLEGVAGG

SEQ ID NO: 28 - protein, 64 aa, Artificial Sequence
  Leader
MSEEQLKAFIAKVQADSSLQEQLKAEGADPVSIAKAAGFTITTEDLNSHRQNLSDEELEGAAGG

SEQ ID NO: 29 - protein, 64 aa, Artificial Sequence
  Leader
MSEEQLKAFIAKVQADTSLQEQLKAEGADVVAIAKAAGFAITKEDLNSHRQTLSEDELESVAGG

SEQ ID NO: 30 - protein, 64 aa, Artificial Sequence
  Leader
MSEEQLKAFIAKVQADASLQEQLRTEGADVVAIAKAAGFSITTEDLNSHRQNLSDDELEGVAGG

SEQ ID NO: 31 - protein, 64 aa, Artificial Sequence
  Leader
MSEEQLKAFIAKVQGDSSLQEQLKAEGADVVAIAKAAGFTIKQQDLNAAASELSDEELEAASGG

SEQ ID NO: 32 - protein, 64 aa, Artificial Sequence
  Leader
MSEEQLKAFIAKVQGDTSLQEQLKAEGADVVAIAKAAGFSITTEDLNTHRQTLSDRELEGVAGG

SEQ ID NO: 33 - protein, 82 aa, Artificial Sequence
  Leader
MLLAEETIELSIPFFQMSEEQLKAFLEKVKADTSLQEKLKAAADSDAVLVIAKDAGFSISADDLKNAQSEISEEELESVAGG

SEQ ID NO: 34 - protein, 66 aa, Artificial Sequence
  Leader
MSEEQLKAFLEKVKADTSLQEKLKAAADSDAVLVIAKDAGFSISADDLKNAQSEISEEELESVAGG

SEQ ID NO: 35 - protein, 71 aa, Artificial Sequence
  Leader
MSEEQLKALIAKVQADTSLQEQLKAEGADVVAIAKAAGFSITTEDLKEHRQTLSVGRQTLSESELEGLAGG

SEQ ID NO: 36 - protein, 64 aa, Artificial Sequence
  Leader
MSEEQLKAFIAKVQADTSLQEQLKAEGADPVAIAKAAGFSITTEDLKEHRQALSDDDLEGVAGG

SEQ ID NO: 37 - protein, 64 aa, Artificial Sequence
  Leader
MSEEQLKAFIAKVQADTSLQEQLKAEGADVVAIAKAAGFTITTEDLNSHRQNLTDDELEGVAGG

SEQ ID NO: 38 - protein, 84 aa, Artificial Sequence
  Leader
MHSSALGWAKIRKSDATKMSEEQLKAFLEKVKGDTSLQEKLQAAADSDAVLAIAKEAGFMISADELKKAQSEISEEELESAAGG

SEQ ID NO: 39 - protein, 66 aa, Artificial Sequence
  Leader
MSEEQLKAFLEKVKGDTSLQEKLQAAADSDAVLAIAKEAGFMISADELKKAQSEISEEELESAAGG

SEQ ID NO: 40 - protein, 82 aa, Artificial Sequence; identical to SEQ ID NO: 33
  Leader

SEQ ID NO: 41 - protein, 66 aa, Artificial Sequence
  Leader
MSEEQLKAFLEKVKADTSLQEKLKAAAGSDAVLAIAKAAGLMISADDLTKAQSEISDAELEDAAGG

SEQ ID NO: 42 - protein, 66 aa, Artificial Sequence
  Leader
MSEEQLKAFIEKVKGDTSLQEKLKAAADTNAVSAIAKEAGFSISADDLTKAQSEISDEELEGVAGG

SEQ ID NO: 43 - protein, 66 aa, Artificial Sequence
  Leader
MSEEQLKAFLEKVKKDTNLQEKLKAAADSYAVLAIAKEAGFMISADDLKNAQSELSDQELEGAAGG

SEQ ID NO: 44 - protein, 66 aa, Artificial Sequence
  Leader
MSEEQLKAFLEKVKADTSLQEKLKAAADANAVAAIAKESGFSISVEDINKAQSELSDKELEGVSGG

SEQ ID NO: 45 - protein, 64 aa, Artificial Sequence
  Leader
MSEEQLKAFLAKVQADASLQEQLKAEGSDVIAIAKAAGFVITKEDLKAHRQNLSEDELEGVAGG

SEQ ID NO: 46 - protein, 66 aa, Artificial Sequence
  Leader
MSEEQLKAFIEKVQGDTSLQEKLKAVADADAVAAIAKEAGFSISADDLTKDQSELSDEELEGVAGG

SEQ ID NO: 47 - protein, 64 aa, Artificial Sequence
  Leader
MSEEQLKAFIAKVQADTSLQEQLKAEDADPVAIAKAAGFSITTEDLKEHRQNLSEKDLEGVAGG

SEQ ID NO: 48 - protein, 64 aa, Artificial Sequence
  Leader
MSEEQLKAFIAKVQADSSLQEQLKAEGADVVAIAKAAGFSITTEDLNTHRQNLSDDELEDVAGG

SEQ ID NO: 49 - protein, 64 aa, Artificial Sequence
  Leader
MSEEQLKAFIAKVQADSSLQEQLKAEGADPVAIAKAAGFAITTEDLQANQQNLSDQELEGAAGG

SEQ ID NO: 50 - protein, 72 aa, Artificial Sequence
  Leader
MKHRQLNLMSEEQLKAFIAKVQADASLQEQLKAEGADVVVIAKAAGFAITKEDLNTHRQTLSDNELEGVAGG

SEQ ID NO: 51 - protein, 64 aa, Artificial Sequence
  Leader
MSEEQLKAFIAKVQADASLQEQLKAEGADVVVIAKAAGFAITKEDLNTHRQTLSDNELEGVAGG

SEQ ID NO: 52 - protein, 66 aa, Artificial Sequence
  Leader
MSEEQLKAFLEKVQGDTSLQEQLKAAADTDAVAAIAKEAGFSISAEDLTQAQLELSEEELESVAGG

SEQ ID NO: 53 - protein, 64 aa, Artificial Sequence
  Leader
MSEEQLKAFIAKVQADSSLQEQLKAEGADPVAIAKAAGFTFTTEDLTLYRQNLSDDELEGVAGG

SEQ ID NO: 54 - protein, 68 aa, Artificial Sequence
  Leader
MSEEQLKAFIAKVQADASLQEQLKAEGADPIAIAKVAGFSITQADLEANWKESSRELSDADLENVSGG

SEQ ID NO: 55 - protein, 43 aa, Artificial Sequence
  Leader
MKITDADSIASIAKEAGFEITADEVKKAQSTLPDEPLDLDAGG

SEQ ID NO: 56 - protein, 66 aa, Artificial Sequence
  Leader
MSEEQLKAFLEKVKADTSLQEKLKAASDADAVVAIAKAAGFTITTEDLNSHRQNLTEDELEGVAGG

SEQ ID NO: 57 - protein, 66 aa, Artificial Sequence
  Leader
MSEEQLKAFLEKVKADTSVQEKLKAASDADAVVAIAKAAGFTITTEDLNSHRQNLTEDELEGVAGG

SEQ ID NO: 58 - protein, 66 aa, Artificial Sequence
  Leader
MSEEQLKAFLEKVKGDTSLQEKLKAAADSDAVLAIAKAAGFTITTEDLNSHRQNLTEDELEGVAGG

SEQ ID NO: 59 - protein, 66 aa, Artificial Sequence
  Leader
MSEEQLKAFLEKVKGDSSLQEKLKAAADNDAVTAIAKEAGFSISADDLKNAQSELSDEELEGVAGG

SEQ ID NO: 60 - protein, 66 aa, Artificial Sequence; identical to SEQ ID NO: 59
  Leader - Ident SEQ ID 59

SEQ ID NO: 61 - protein, 66 aa, Artificial Sequence
  Leader
MSEEQLKAFLEKVKADTTLQEKLKAAADSDAVLAIAKEAGFSISADDLKNAQSEVSEEELEGVAGG

SEQ ID NO: 62 - protein, 66 aa, Artificial Sequence
  Leader
MSEEQLKAFLEKVKGDTSLQEKLKAAADADAVTAIANEAGFSISANDLTKLQSELSDEELEGVAGG

SEQ ID NO: 63 - protein, 65 aa, Artificial Sequence
  Leader
MQKRLEAFLEKVKADTSLQEKLKAAADTNAVVAIAKDAGFSISADDLTKAQPTLSEEELEGVAGG

SEQ ID NO: 64 - protein, 66 aa, Artificial Sequence
  Leader
MSEEQLKAFLEKVKADTGLQEKLKAASDANAVAAIAQEAGFSISADDLKKAESEIVEEVLEGVAGG

SEQ ID NO: 65 - protein, 67 aa, Artificial Sequence
  Leader
MSEEQLKAFLEKVKADTSLQEKLKAASDANAASAIAKEAGFSISADEFTKAPSTELSAEELEGVAGG

SEQ ID NO: 66 - protein, 66 aa, Artificial Sequence
  Leader
MSEEQLKAFLEILKNDTSLQEKLKAAADADAVLAIAKEAGFIVSPDDLFKAQSELSDQDMEGVAGG

SEQ ID NO: 67 - protein, 2 aa, Artificial Sequence
  Linker
GS

SEQ ID NO: 68 - protein, 10 aa, Artificial Sequence
  Linker
GSGSGSGSGS

SEQ ID NO: 69 - protein, 20 aa, Artificial Sequence
  Linker
GSGSGSGSGSGSGSGSGSGS

SEQ ID NO: 70 - protein, 30 aa, Artificial Sequence
  Linker
GSGSGSGSGSGSGSGSGSGSGSGSGSGSGS

SEQ ID NO: 71 - protein, 5 aa, Artificial Sequence
  Linker
GGGGS

SEQ ID NO: 72 - protein, 10 aa, Artificial Sequence
  Linker
GGGGGGGGGS

SEQ ID NO: 73 - protein, 15 aa, Artificial Sequence
  Linker
GGGGGGGGGGGGGGS

SEQ ID NO: 74 - protein, 324 aa, Planktothrix agardhii
MSLSRDVVLLITHSGDFFTIDRVIEALLKKGVQPFRFDTDQFPLNVQLTANFGKSNSDHQLEYNGNSISTKQVQAVWLRRIWEPDFQELDPQYQQACAKESKATLNGFWDSLRDANWVDHLAQIEAASNKLLQLRLASEVGFTIPQTLITNKAEAAREFFQQVNGKMVSKLLTALSRSMESTSFFLYTSLVKEEYLQDAESLRYCPMVFQEQIFKQQELRVIYVNGKVFVGALNASVYEDSTVDWRAPGIEVGAWQNHQLPEELIRRLQTFMGELGLLFGAFDFILTPSGDYVFLEVNPGGEWGMLERDLDLPISQAIADTLIP

SEQ ID NO: 75 - protein, 330 aa, Planktothrix agardhii
MTVLIFTFSNDNESIPLVVKAIEAKGEKAFRFDTDRFPTEVKLDIYSGDFEGGIVTDGEHKLDLSEVSSVWYRRMRYGAKLPDSMDQQFREASLKECRLTVRGLIASLPGFHFDPMINVDRANNKQLQLQIARQLGLLTPRTLTSNNPEAVKQFAQDCKEQGIVTKMLSQFAIYGENKEENVVFTSPVTDDDLDNLEGLSFCPMTFQENVPKALELRTIIVGKQVFTAAINSQQLDGATYDWRKEGRALHEKWQAYNLPEDIEKKLLQLTAYFGLNYGAIDIIVTPDGRHVFLEINPVGEFFWLELFAPYFPISQAIAEILLTPTNQKPF

SEQ ID NO: 76 - protein, 34 aa, Planktothrix agardhii
MSKNVKVSAPKAVPFFARFLAEQAVEANNSNSAP

SEQ ID NO: 77 - protein, 324 aa, Planktothrix prolifica
MSLSRDVVLLITHSGDFFTIDRVIEALLKKGVQPFRFDTDQFPLNVQLTANFGKLKSDHQLEYNGHSISTKQVQAVWLRRIWEPDFQELDPQYQQACAKESKATLNGFWDSLREANWVDHLAKIEAASNKLLQLRLASELGFTIPQTLITNKAEAAREFFQQVNGKMVSKLLTALSRSMESTSFFLYTSLVKEEDLQDAESLRYCPMVFQEQISKQQELRVIYVNGKVFVGALNASVYEDSTVDWRSPGIEVGAWQNHQLPEELIRRLQTFMGELGLLFGAFDFILTPSGDYVFLEVNPGGEWGMLERDLNLPISQAIADTLIP

SEQ ID NO: 78 - protein, 330 aa, Planktothrix prolifica
MTVLIFTFSNDNESIPLVVKAIEAKGEKAFRFDTDRFPTEVKLDIYSGDFEGGIVTDGEQKLDLSEVSSVWYRRMRYGAKLPDSMDQQFREASLKECRLTVRGLIASLPGFHFDPMINVDRANNKQLQLQIARQLGLLTPRTLTSNNPEAVKQFAQECKEQGIVTKMLSQFAIYGENKEENVVFTSPVTDDDLDNLEGLSFCPMTFQENVPKALELRTIIVGKQVFTAAINSQQLDGATYDWRKEGRALTEKWQTYNLPEDIQKKLLQLTAYFGLNYGAIDIIVTPDGRHVFLEINPVGEFFWLELFSPYFPISQAIAEILLTPTNHLSD

SEQ ID NO: 79 - protein, 36 aa, Planktothrix prolifica
MSKNIKVSTGSAVPFFARFLSEQDTETGDSTSTDIP

SEQ ID NO: 80 - protein, 325 aa, Microcystis aeruginosa
  misc_feature (250)..(250): Xaa can be any naturally occurring amino acid
MKESPKVVLLLTHSGDFFTIDRVAEAIEKKGATPFRLDTDKFPLEVQLAAQFNGKKSFYQLTYNHQSIDSEQVQSVWTRRIWQPEITGDLDPQFREACVRESQTTLAGFWDSLRSARWLDNLAQIERAKNKLLQLRLASEVGLIIPPTLVTNNPDAAREFFSQVQGRMVSKLLTAIARSMESPEFFLYTSRVKAEDLEEAESLRYCPMVFQAEIPKQLELRVVVVNGQTFVGALESSQYNNSAVDWRRPXIDPGAWQHHTLPDSLLQQLQIFMANLGLNFGAFDFILTPGGEYVFLEVNPGGEWGMLERDLDLPISNAIADFLVC

SEQ ID NO: 81 - protein, 324 aa, Microcystis aeruginosa
MTVLIVTFSHDNESIPLVIKAIETMGKKAFRFDTDRFPTEVKVDLYSGGQKGGIITDGDQKLELKEVSAVWYRRMRYGLKLPDGMDSQFREASLKECRLSIRGMIASLSGFHLDPIAKVDHANHKQLQLQVAQQLGLLIPGTLTSNNPEAVKQFAQEFEATGIVTKMLSQFAIYGDKQEEMVVFTSPVTKEDLDNLEGLQFCPMTFQENIPKALELRITIVGEQIFTAAINSQQLDGAIYDWRKEGRALHQQWQPYDLPKTIEKQLLELVKYFGLNYGAIDMIVTPDERYIFLEINPVGEFFWLELYPPYFPISQAIAEVLVNS

SEQ ID NO: 82 - protein, 35 aa, Microcystis aeruginosa
AYPNDQQGKALPFFARFLSVSKEESSIKSPSPEPT

SEQ ID NO: 83 - protein, 1174 aa, Artificial Sequence
  Enzyme-Leader-Fusion
MKHRQLNLMSEEQLKAFIAKVQADTSLQEQLKVEGADVVAIAKASGFAITTEDLKAHQANSQKNLSDAELEGVAGGGSGSGSGSGSGSGSGSGSGSGSGSGSGSGSMESPSSWKTSWLAAIAPDEPHKFDRRLEWDELSEENFFAALNSEPASLEEDDPCFEEALQDALEALKAAWDLPLLPVDNNLNRPFVDVWWPIRCHSAESLRQSFVSDSAGLADEIFDQLADSLLDRLCALGDQVLWEAFNKERTPGTMLLAHLGAAGDGSGPPVREHYERFIQSHRRNGLAPLLKEFPVLGRLIGTVLSLWFQGSVEMLQRICADRTVLQQCFAIPCGHHLKTVKQGLSDPHRGGRAVAVLEFADPNSTANSSMHVVYKPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAEVKQMQHLDIPFFTHLIDADALQLGGLEQELPGFIQTSGLAAAYERLRNLDTDEIAFQLRLIRGAVEARELHTTPESSPTLPPPATPEALMSSSAETSLEAAKRIAHRLLELAIRDSQGQVEWLGMDLGADGESFSFGPVGLSLYGGSIGIAHLLQRLQAQQVSLMDADAIQTAILQPLVGLVDQPSDDGRRRWWRDQPLGLSGCGGTLLALTLQGEQAMANSLLAAALPRFIEADQQLDLIGGCAGLIGSLVQLGTESALQLALRAGDHLIAQQNEEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCCGSLGLMVLLEMLSAGPWPIDNQLRSHCQDVAFQYRLQALQRCSAEPIKLRCFGTKEGLLVLPGFFTGLSGMGLALLEDDPSRAVVSQLISAGLWPTE

SEQ ID NO: 84 - protein, 1174 aa, Artificial Sequence
  Enzyme-Leader-Fusion
MESPSSWKTSWLAAIAPDEPHKFDRRLEWDELSEENFFAALNSEPASLEEDDPCFEEALQDALEALKAAWDLPLLPVDNNLNRPFVDVWWPIRCHSAESLRQSFVSDSAGLADEIFDQLADSLLDRLCALGDQVLWEAFNKERTPGTMLLAHLGAAGDGSGPPVREHYERFIQSHRRNGLAPLLKEFPVLGRLIGTVLSLWFQGSVEMLQRICADRTVLQQCFAIPCGHHLKTVKQGLSDPHRGGRAVAVLEFADPNSTANSSMHVVYKPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAEVKQMQHLDIPFFTHLIDADALQLGGLEQELPGFIQTSGLAAAYERLRNLDTDEIAFQLRLIRGAVEARELHTTPESSPTLPPPATPEALMSSSAETSLEAAKRIAHRLLELAIRDSQGQVEWLGMDLGADGESFSFGPVGLSLYGGSIGIAHLLQRLQAQQVSLMDADAIQTAILQPLVGLVDQPSDDGRRRWWRDQPLGLSGCGGTLLALTLQGEQAMANSLLAAALPRFIEADQQLDLIGGCAGLIGSLVQLGTESALQLALRAGDHLIAQQNEEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCCGSLGLMVLLEMLSAGPWPIDNQLRSHCQDVAFQYRLQALQRCSAEPIKLRCFGTKEGLLVLPGFFTGLSGMGLALLEDDPSRAVVSQLISAGLWPTEGSGSGSGSGSGSGSGSGSGSGSGSGSGSGSMKHRQLNLMSEEQLKAFIAKVQADTSLQEQLKVEGADVVAIAKASGFAITTEDLKAHQANSQKNLSDAELEGVAGG

SEQ ID NO: 85 - protein, 1174 aa, Artificial Sequence
  Enzyme-Leader-Fusion
MESPSSWKTSWLAAIAPDEPHKFDRRLEWDELSEENFFAALNSEPASLEEDDPCFEEALQDALEALKAAWDLPLLPVDNNLNRPFVDVWWPIRCHSAESLRQSFVSDSAGLADEIFDQLADSLLDRLCALGDQVLWEAFNKERTPGTMLLAHLGAAGDGSGPPVREHYERFIQSHRRNGLAPLLKEFPVLGRLIGTVLSLWFQGSVEMLQRICADRTVLQQCFAIPCGHHLKTVKQGLSDPHRGGRAVAVLEFADPNSTANSSMHVVYKPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAEVKQMQHLDIPFFTHLIDADALQLGGLEQELPGFIQTSGLAAAYERLRNLDTDEIAFQLRLIRGAVEARELHTTPESSPTLPPPATPEALMSSSAETSLEAAKRIAHRLLELAIRDSQGQVEWLGMDLGADGESFSFGPVGLSLYGGSGSGSGSGSGSGSGSGSGSGSGSGSGSGSMKHRQLNLMSEEQLKAFIAKVQADTSLQEQLKVEGADVVAIAKASGFAITTEDLKAHQANSQKNLSDAELEGVAGGGSIGIAHLLQRLQAQQVSLMDADAIQTAILQPLVGLVDQPSDDGRRRWWRDQPLGLSGCGGTLLALTLQGEQAMANSLLAAALPRFIEADQQLDLIGGCAGLIGSLVQLGTESALQLALRAGDHLIAQQNEEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCCGSLGLMVLLEMLSAGPWPIDNQLRSHCQDVAFQYRLQALQRCSAEPIKLRCFGTKEGLLVLPGFFTGLSGMGLALLEDDPSRAVVSQLISAGLWPTE

SEQ ID NO: 86 - protein, 1162 aa, Artificial Sequence
  Enzyme-Leader-Fusion
MSEEQLKAFIAKVQADSSLQEQLKAEGADVVAIAKAAGFSITTEDLNTHRQNLSDDELEDVAGGGSGSGSGSGSGSGSGSGSGSGSGSGSGSGSMTSPTSWKTSWLAAIAPDEPHKFDRRLEWDELSEENFFAALNSAPTSLEEDDPCFDEALQDALEALKAAWDLPLLPVDNTVNRPFVDLWWPIRCHSAESLRQIFVSDSAGLADEIFDQLADSLLDRLCALGDQVLWEAFNKERTPGTMLLAHLGAAGDGSGPPVREHYERFIQSHRRNGLAPLLKEFPVLGRLIDTVLSLWFQGSVEMLQRICADRTVLQQGFAIPCGHHLKTVKQGLSDPHRGGRAVAVLEFADPNSTANSSMHVVYKPKDMAVDAAYQATLADLNAHSDLSPLRTLAIHNGDGYGYMEHVVHHFCANDKELTNFYFNAGRLTALLHLLGCTDCHHENLIACGDQLLLIDTETLLEADLPDHISDASSTTAQPEPSTLQRQFQRSVLRSGLLPQWMFLGESKQAIDISALGISPPNKPERIAAGWLGFNSDGMMPGRVSQPVEIPTSLPVGIADVNPFDRFLEDFCDGFSMQSKALIKLRNRWLDLNGILAHFAGLPRRIVLRATRVYFNIQRQQLEPTALRSSLAQALKLEQLARSFLLAESKPLHWPIFAAEVKQMQQLDIPFFTHLIDADALQLGGLEQELPGFIQTSGLAAAYERLRNLDSNEIAFQLRLIHGAVEARELHTTPESSPTLHPPATPEALMSASAETSLKAAKRIAHRLLELAIRDSQGQVEWLGMDLGADGESFAFGPVGLSLYGGSIGISHLLQRLQAQQVPLMDADAIQTAILQPLVGLVAQPSDDGRRRWWRDQPLGLSGCGGTLLALALQGEQAMANSLLAAALPRFIEADQQLDLIGGCAGLIGSLVQLGTESALQWALRAGDHLIAQQNEEGAWSSSSSQPALLGFSHGVAGYAAALAHLHAFSGDERYRIAAAAALAYERARFNKDAGNWPDYRSISRDSDSDEPSFMASWCHGAPGIALGRACLWGTALWDEECTKEIGIGLQTTAAVSAVSTDHLCCGSLGLMVLLEMLSKGPWPIDNQLRSHCQDVASQYRLQALQRCSAEPIKLRCFGTKEGLLVLPGFFTGLSGMGLALLEDDPSRAVVSQLISAGLWPTE

SEQ ID NO: 87 - protein, 1162 aa, Artificial Sequence
  Enzyme-Leader-Fusion
MTSPTSWKTSWLAAIAPDEPHKFDRRLEWDELSEENFFAALNSAPTSLEEDDPCFDEALQDALEALKAAWDLPLLPVDNTVNRPFVDLWWPIRCHSAESLRQIFVSDSAGLADEIFDQLADSLLDRLCALGDQVLWEAFNKERTPGTMLLAHLGAAGDGSGPPVREHYERFIQSHRRNGLAPLLKEFPVLGRLIDTVLSLWFQGSVEMLQRICADRTVLQQGFAIPCGHHLKTVKQGLSDPHRGGRAVAVLEFADPNSTANSSMHVVYKPKDMAVDAAYQATLADLNAHSDLSPLRTLAIHNGDGYGYMEHVVHHFCANDKELTNFYFNAGRLTALLHLLGCTDCHHENLIACGDQLLLIDTETLLEADLPDHISDASSTTAQPEPSTLQRQFQRSVLRSGLLPQWMFLGESKQAIDISALGISPPNKPERIAAGWLGFNSDGMMPGRVSQPVEIPTSLPVGIADVNPFDRFLEDFCDGFSMQSKALIKLRNRWLDLNGILAHFAGLPRRIVLRATRVYFNIQRQQLEPTALRSSLAQALKLEQLARSFLLAESKPLHWPIFAAEVKQMQQLDIPFFTHLIDADALQLGGLEQELPGFIQTSGLAAAYERLRNLDSNEIAFQLRLIHGAVEARELHTTPESSPTLHPPATPEALMSASAETSLKAAKRIAHRLLELAIRDSQGQVEWLGMDLGADGESFAFGPVGLSLYGGSIGISHLLQRLQAQQVPLMDADAIQTAILQPLVGLVAQPSDDGRRRWWRDQPLGLSGCGGTLLALALQGEQAMANSLLAAALPRFIEADQQLDLIGGCAGLIGSLVQLGTESALQWALRAGDHLIAQQNEEGAWSSSSSQPALLGFSHGVAGYAAALAHLHAFSGDERYRIAAAAALAYERARFNKDAGNWPDYRSISRDSDSDEPSFMASWCHGAPGIALGRACLWGTALWDEECTKEIGIGLQTTAAVSAVSTDHLCCGSLGLMVLLEMLSKGPWPIDNQLRSHCQDVASQYRLQALQRCSAEPIKLRCFGTKEGLLVLPGFFTGLSGMGLALLEDDPSRAVVSQLISAGLWPTEGSGSGSGSGSGSGSGSGSGSGSGSGSGSGSMSEEQLKAFIAKVQADSSLQEQLKAEGADVVAIAKAAGFSITTEDLNTHRQNLSDDELEDVAGG

SEQ ID NO: 88 - protein, 1162 aa, Artificial Sequence
  Enzyme-Leader-Fusion
MTSPTSWKTSWLAAIAPDEPHKFDRRLEWDELSEENFFAALNSAPTSLEEDDPCFDEALQDALEALKAAWDLPLLPVDNTVNRPFVDLWWPIRCHSAESLRQIFVSDSAGLADEIFDQLADSLLDRLCALGDQVLWEAFNKERTPGTMLLAHLGAAGDGSGPPVREHYERFIQSHRRNGLAPLLKEFPVLGRLIDTVLSLWFQGSVEMLQRICADRTVLQQGFAIPCGHHLKTVKQGLSDPHRGGRAVAVLEFADPNSTANSSMHVVYKPKDMAVDAAYQATLADLNAHSDLSPLRTLAIHNGDGYGYMEHVVHHFCANDKELTNFYFNAGRLTALLHLLGCTDCHHENLIACGDQLLLIDTETLLEADLPDHISDASSTTAQPEPSTLQRQFQRSVLRSGLLPQWMFLGESKQAIDISALGISPPNKPERIAAGWLGFNSDGMMPGRVSQPVEIPTSLPVGIADVNPFDRFLEDFCDGFSMQSKALIKLRNRWLDLNGILAHFAGLPRRIVLRATRVYFNIQRQQLEPTALRSSLAQALKLEQLARSFLLAESKPLHWPIFAAEVKQMQQLDIPFFTHLIDADALQLGGLEQELPGFIQTSGLAAAYERLRNLDSNEIAFQLRLIHGAVEARELHTTPESSPTLHPPATPEALMSASAETSLKAAKRIAHRLLELAIRDSQGQVEWLGMDLGADGESFAFGPVGLSLYGGSGSGSGSGSGSGSGSGSGSGSGSGSGSGSMSEEQLKAFIAKVQADSSLQEQLKAEGADVVAIAKAAGFSITTEDLNTHRQNLSDDELEDVAGGGSIGISHLLQRLQAQQVPLMDADAIQTAILQPLVGLVAQPSDDGRRRWWRDQPLGLSGCGGTLLALALQGEQAMANSLLAAALPRFIEADQQLDLIGGCAGLIGSLVQLGTESALQWALRAGDHLIAQQNEEGAWSSSSSQPALLGFSHGVAGYAAALAHLHAFSGDERYRIAAAAALAYERARFNKDAGNWPDYRSISRDSDSDEPSFMASWCHGAPGIALGRACLWGTALWDEECTKEIGIGLQTTAAVSAVSTDHLCCGSLGLMVLLEMLSKGPWPIDNQLRSHCQDVASQYRLQALQRCSAEPIKLRCFGTKEGLLVLPGFFTGLSGMGLALLEDDPSRAVVSQLISAGLWPTE

SEQ ID NO: 89 - protein, 1154 aa, Artificial Sequence
  Enzyme-Leader-Fusion
MSEEQLKAFLEKVKGDSSLQEKLKAAADNDAVTAIAKEAGFSISADDLKNAQSELSDEELEGVAGGGSGSGSGSGSGSGSGSGSGSGSGSGSGSGSMRENWKQQWLDIVAPDEPHKLSRRLDWDELTEEEFREHLSTSPTKANRFEGHWQNCLNECRLAIQKAWEQPLLPISPDNQQRPFEDLWRPICNEAREILKKRLSRLDGVGEGIFEQLTNSLLNRLCSIGEQVLWEKFNSERSPGVMLLAHLGSNGDGSGPPVREGYEQFIRNHRQDGLTQLLGEFPELGRLIGTIVLLWLEGSQEMLERIDQDRQTLETLFGIPKNAPLQTIHQGLSDPHRGGRAVAILGFAGPGEEQYKLVYKPKDMAVDAAYQDLLKDINRSSSDKPLKILKIHNGEGYGYMEYVSHKPARDDKELGFFYKNAGRLTAVLHLLGCTDCHHENLIACGDQLLLIDTETLLEAELPNHINNDTSEAPELSALQKRCQRSILRSGLMPQWMFLGSEKIAIDISALGISPPKQETQKTSGWLGLNSDGMMPGVIERNADLPTSLPIGIGSPNPFSLHLDTFCEGFREQNQTLIEQKDQWLKEGTFLNRFKGLRRRIVLRATRVYFAIQRQQLEPAALRSPLIQAIKLEQLSRSFLLAEQKPLHWPVFAAELEQMQQLDIPFFTHQIDSDALDLNQAQTQLPGYIETSGLSAAKERLEQLDTSEVEFQIKLIRGTCNAQKIRRGASVETSVHSAQENQYTTQTKSAADHQVAATNVMNCLADLAIEDNSGQMEWLGMDLGADGESFSFGPVGFSLYGGSSGVAILDHTLQRLNGTNSQLSAIQSGVLQSIQQLAEKASEDQRLRWWRDQSLGLSGCGGMLLTLQCLGQTSWAQSLLKSGRQRFLEADRQLDLIGGCTGLIGPLLHLKSPEALDLAIHAGKHLLEQQQEDGSWPGRAKNTPGLLGFSHGTAGHAAALAKLHQATHDNRFLEGAKAALHHERAHFDSKEENWPDFRDITQSGSFMTSWCHGAPGIGIARACLWQTELWDEQCRDEIHAAIRTTSKSIGQLRSDHLCCGKAGLAIILEMLCDGPWGVEAHVADLARERTIQIHTSLMNRTSSKMPDLICFAAKETSLVLPGFFTGLSGIGMALLRDDQSKQTTRRLLTAGLWTE

SEQ ID NO: 90 - protein, 1154 aa, Artificial Sequence
  Enzyme-Leader-Fusion
MRENWKQQWLDIVAPDEPHKLSRRLDWDELTEEEFREHLSTSPTKANRFEGHWQNCLNECRLAIQKAWEQPLLPISPDNQQRPFEDLWRPICNEAREILKKRLSRLDGVGEGIFEQLTNSLLNRLCSIGEQVLWEKFNSERSPGVMLLAHLGSNGDGSGPPVREGYEQFIRNHRQDGLTQLLGEFPELGRLIGTIVLLWLEGSQEMLERIDQDRQTLETLFGIPKNAPLQTIHQGLSDPHRGGRAVAILGFAGPGEEQYKLVYKPKDMAVDAAYQDLLKDINRSSSDKPLKILKIHNGEGYGYMEYVSHKPARDDKELGFFYKNAGRLTAVLHLLGCTDCHHENLIACGDQLLLIDTETLLEAELPNHINNDTSEAPELSALQKRCQRSILRSGLMPQWMFLGSEKIAIDISALGISPPKQETQKTSGWLGLNSDGMMPGVIERNADLPTSLPIGIGSPNPFSLHLDTFCEGFREQNQTLIEQKDQWLKEGTFLNRFKGLRRRIVLRATRVYFAIQRQQLEPAALRSPLIQAIKLEQLSRSFLLAEQKPLHWPVFAAELEQMQQLDIPFFTHQIDSDALDLNQAQTQLPGYIETSGLSAAKERLEQLDTSEVEFQIKLIRGTCNAQKIRRGASVETSVHSAQENQYTTQTKSAADHQVAATNVMNCLADLAIEDNSGQMEWLGMDLGADGESFSFGPVGFSLYGGSSGVAILDHTLQRLNGTNSQLSAIQSGVLQSIQQLAEKASEDQRLRWWRDQSLGLSGCGGMLLTLQCLGQTSWAQSLLKSGRQRFLEADRQLDLIGGCTGLIGPLLHLKSPEALDLAIHAGKHLLEQQQEDGSWPGRAKNTPGLLGFSHGTAGHAAALAKLHQATHDNRFLEGAKAALHHERAHFDSKEENWPDFRDITQSGSFMTSWCHGAPGIGIARACLWQTELWDEQCRDEIHAAIRTTSKSIGQLRSDHLCCGKAGLAIILEMLCDGPWGVEAHVADLARERTIQIHTSLMNRTSSKMPDLICFAAKETSLVLPGFFTGLSGIGMALLRDDQSKQTTRRLLTAGLWTEGSGSGSGSGSGSGSGSGSGSGSGSGSGSGSMSEEQLKAFLEKVKGDSSLQEKLKAAADNDAVTAIAKEAGFSISADDLKNAQSELSDEELEGVAGG

SEQ ID NO: 91 - protein, 1154 aa, Artificial Sequence
  Enzyme-Leader-Fusion
MRENWKQQWLDIVAPDEPHKLSRRLDWDELTEEEFREHLSTSPTKANRFEGHWQNCLNECRLAIQKAWEQPLLPISPDNQQRPFEDLWRPICNEAREILKKRLSRLDGVGEGIFEQLTNSLLNRLCSIGEQVLWEKFNSERSPGVMLLAHLGSNGDGSGPPVREGYEQFIRNHRQDGLTQLLGEFPELGRLIGTIVLLWLEGSQEMLERIDQDRQTLETLFGIPKNAPLQTIHQGLSDPHRGGRAVAILGFAGPGEEQYKLVYKPKDMAVDAAYQDLLKDINRSSSDKPLKILKIHNGEGYGYMEYVSHKPARDDKELGFFYKNAGRLTAVLHLLGCTDCHHENLIACGDQLLLIDTETLLEAELPNHINNDTSEAPELSALQKRCQRSILRSGLMPQWMFLGSEKIAIDISALGISPPKQETQKTSGWLGLNSDGMMPGVIERNADLPTSLPIGIGSPNPFSLHLDTFCEGFREQNQTLIEQKDQWLKEGTFLNRFKGLRRRIVLRATRVYFAIQRQQLEPAALRSPLIQAIKLEQLSRSFLLAEQKPLHWPVFAAELEQMQQLDIPFFTHQIDSDALDLNQAQTQLPGYIETSGLSAAKERLEQLDTSEVEFQIKLIRGTCNAQKIRRGASVETSVHSAQENQYTTQTKSAADHQVAATNVMNCLADLAIEDNSGQMEWLGMDLGADGESFSFGPVGFSLYGGSGSGSGSGSGSGSGSGSGSGSGSGSGSGSMSEEQLKAFLEKVKGDSSLQEKLKAAADNDAVTAIAKEAGFSISADDLKNAQSELSDEELEGVAGGGSSGVAILDHTLQRLNGTNSQLSAIQSGVLQSIQQLAEKASEDQRLRWWRDQSLGLSGCGGMLLTLQCLGQTSWAQSLLKSGRQRFLEADRQLDLIGGCTGLIGPLLHLKSPEALDLAIHAGKHLLEQQQEDGSWPGRAKNTPGLLGFSHGTAGHAAALAKLHQATHDNRFLEGAKAALHHERAHFDSKEENWPDFRDITQSGSFMTSWCHGAPGIGIARACLWQTELWDEQCRDEIHAAIRTTSKSIGQLRSDHLCCGKAGLAIILEMLCDGPWGVEAHVADLARERTIQIHTSLMNRTSSKMPDLICFAAKETSLVLPGFFTGLSGIGMALLRDDQSKQTTRRLLTAGLWTE

SEQ ID NO: 92 - protein, 388 aa, Artificial Sequence
  Enzyme-Leader-Fusion
MSKNVKVSAPKAVPFFARFLAEQAVEANNSNSAPGSGSGSGSGSGSGSGSGSGSGSGSGSGSGSMSLSRDVVLLITHSGDFFTIDRVIEALLKKGVQPFRFDTDQFPLNVQLTANFGKSNSDHQLEYNGNSISTKQVQAVWLRRIWEPDFQELDPQYQQACAKESKATLNGFWDSLRDANWVDHLAQIEAASNKLLQLRLASEVGFTIPQTLITNKAEAAREFFQQVNGKMVSKLLTALSRSMESTSFFLYTSLVKEEYLQDAESLRYCPMVFQEQIFKQQELRVIYVNGKVFVGALNASVYEDSTVDWRAPGIEVGAWQNHQLPEELIRRLQTFMGELGLLFGAFDFILTPSGDYVFLEVNPGGEWGMLERDLDLPISQAIADTLIP

SEQ ID NO: 93 - protein, 388 aa, Artificial Sequence
  Enzyme-Leader-Fusion
MSLSRDVVLLITHSGDFFTIDRVIEALLKKGVQPFRFDTDQFPLNVQLTANFGKSNSDHQLEYNGNSISTKQVQAVWLRRIWEPDFQELDPQYQQACAKESKATLNGFWDSLRDANWVDHLAQIEAASNKLLQLRLASEVGFTIPQTLITNKAEAAREFFQQVNGKMVSKLLTALSRSMESTSFFLYTSLVKEEYLQDAESLRYCPMVFQEQIFKQQELRVIYVNGKVFVGALNASVYEDSTVDWRAPGIEVGAWQNHQLPEELIRRLQTFMGELGLLFGAFDFILTPSGDYVFLEVNPGGEWGMLERDLDLPISQAIADTLIPGSGSGSGSGSGSGSGSGSGSGSGSGSGSGSMSKNVKVSAPKAVPFFARFLAEQAVEANNSNSAP

SEQ ID NO: 94 - protein, 388 aa, Artificial Sequence
  Enzyme-Leader-Fusion
MSLSRDVVLLITHSGDFFTIDRVIEALLKKGVQPFRFDTDQFPLNVQLTANFGKSNSDHQLEYNGNSISTKQVQAVWLRRIWEPDFQELDPQYQQACAKESKATLNGFWDSLRDANWVDHLAQIEAGSGSGSGSGSGSGSGSGSGSGSGSGSGSGSMSKNVKVSAPKAVPFFARFLAEQAVEANNSNSAPASNKLLQLRLASEVGFTIPQTLITNKAEAAREFFQQVNGKMVSKLLTALSRSMESTSFFLYTSLVKEEYLQDAESLRYCPMVFQEQIFKQQELRVIYVNGKVFVGALNASVYEDSTVDWRAPGIEVGAWQNHQLPEELIRRLQTFMGELGLLFGAFDFILTPSGDYVFLEVNPGGEWGMLERDLDLPISQAIADTLIP

SEQ ID NO: 95 - protein, 394 aa, Artificial Sequence
  Enzyme-Leader-Fusion
MSKNVKVSAPKAVPFFARFLAEQAVEANNSNSAPGSGSGSGSGSGSGSGSGSGSGSGSGSGSGSMTVLIFTFSNDNESIPLVVKAIEAKGEKAFRFDTDRFPTEVKLDIYSGDFEGGIVTDGEHKLDLSEVSSVWYRRMRYGAKLPDSMDQQFREASLKECRLTVRGLIASLPGFHFDPMINVDRANNKQLQLQIARQLGLLTPRTLTSNNPEAVKQFAQDCKEQGIVTKMLSQFAIYGENKEENVVFTSPVTDDDLDNLEGLSFCPMTFQENVPKALELRTIIVGKQVFTAAINSQQLDGATYDWRKEGRALHEKWQAYNLPEDIEKKLLQLTAYFGLNYGAIDIIVTPDGRHVFLEINPVGEFFWLELFAPYFPISQAIAEILLTPTNQKPF

SEQ ID NO: 96 - protein, 394 aa, Artificial Sequence
  Enzyme-Leader-Fusion
MTVLIFTFSNDNESIPLVVKAIEAKGEKAFRFDTDRFPTEVKLDIYSGDFEGGIVTDGEHKLDLSEVSSVWYRRMRYGAKLPDSMDQQFREASLKECRLTVRGLIASLPGFHFDPMINVDRANNKQLQLQIARQLGLLTPRTLTSNNPEAVKQFAQDCKEQGIVTKMLSQFAIYGENKEENVVFTSPVTDDDLDNLEGLSFCPMTFQENVPKALELRTIIVGKQVFTAAINSQQLDGATYDWRKEGRALHEKWQAYNLPEDIEKKLLQLTAYFGLNYGAIDIIVTPDGRHVFLEINPVGEFFWLELFAPYFPISQAIAEILLTPTNQKPFGSGSGSGSGSGSGSGSGSGSGSGSGSGSGSMSKNVKVSAPKAVPFFARFLAEQAVEANNSNSAP

SEQ ID NO: 97 - protein, 394 aa, Artificial Sequence
  Enzyme-Leader-Fusion
MTVLIFTFSNDNESIPLVVKAIEAKGEKAFRFDTDRFPTEVKLDIYSGDFEGGIVTDGEHKLDLSEVSSVWYRRMRYGAKLPDSMDQQFREASLKECRLTVRGLIASLPGFHFDPMINVDRGSGSGSGSGSGSGSGSGSGSGSGSGSGSGSMSKNVKVSAPKAVPFFARFLAEQAVEANNSNSAPANNKQLQLQIARQLGLLTPRTLTSNNPEAVKQFAQDCKEQGIVTKMLSQFAIYGENKEENVVFTSPVTDDDLDNLEGLSFCPMTFQENVPKALELRTIIVGKQVFTAAINSQQLDGATYDWRKEGRALHEKWQAYNLPEDIEKKLLQLTAYFGLNYGAIDIIVTPDGRHVFLEINPVGEFFWLELFAPYFPISQAIAEILLTPTNQKPF

SEQ ID NO: 98 - protein, 22 aa, Artificial Sequence
  Peptide
TIGGTIVSITCETCDLLVGKMC

SEQ ID NO: 99 - protein, 13 aa, Artificial Sequence
  Peptide
  misc_feature (2)..(2): X=X(n), wherein X is any amino acid and n=3
  misc_feature (4)..(4): X=X(n), wherein X is any amino acid and n=2
  misc_feature (6)..(6): X is any amino acid
  misc_feature (9)..(9): X is any amino acid
  misc_feature (12)..(12): X=X(n), wherein X is any amino acid and n=7
TXTXSXTCXTCXC

SEQ ID NO: 100 - protein, 14 aa, Artificial Sequence
  Peptide
  misc_feature (2)..(2): X=(X)n, wherein X is any amino acid and n=1-100
  misc_feature (4)..(4): X=(X)n, wherein X is any amino acid and n=1-100
  misc_feature (6)..(6): X=(X)n, wherein X is any amino acid and n=1-100
  misc_feature (9)..(9): X=(X)n, wherein X is any amino acid and n=1-100
  misc_feature (12)..(12): X=(X)n, wherein X is any amino acid and n=1-100
  misc_feature (14)..(14): X=(X)n, wherein X is any amino acid and n=1-100
TXTXSXTCXTCXCX

SEQ ID NO: 101 - protein, 19 aa, Artificial Sequence
  Peptide
AACHNHAPSMPPSYWEGEC

SEQ ID NO: 102 - protein, 9 aa, Artificial Sequence
  Peptide
  misc_feature (1)..(1): X=X(n), wherein X is is any amino acid and n=0-100
  misc_feature (3)..(3): X=X(n), wherein X is is any amino acid and n=1-10
  misc_feature (5)..(5): X=X(n), wherein X is is any amino acid and n=0-10
  misc_feature (7)..(7): X=X(n), wherein X is is any amino acid and n=1-10
  misc_feature (9)..(9): X=X(n), wherein X is is any amino acid and n=0-100
XCXSXSXCX

SEQ ID NO: 103 - DNA, 82 bp, Artificial Sequence
  Oligo
ctaaacaactttcaacagtttctgcggccgccccgtgcaccgccatggccggctgggccgcatagaaaggaacaactaaagg

SEQ ID NO: 104 - DNA, 20 bp, Artificial Sequence
  Primer
gcgatggttgttgtcattgt

SEQ ID NO: 105 - DNA, 20 bp, Artificial Sequence
  Primer
attccacagacagccctcat

SEQ ID NO: 106 - protein, 65 aa, Artificial Sequence
  Oligo
CTGCGGCCGCGCCTGCGCASNNSNNSNNGGWCGGAGWSNNSNNSNNGCAGGCCGCGCCGCCCGCC

SEQ ID NO: 107 - protein, 13 aa, Artificial Sequence
  Peptide
  MISC_FEATURE (4)..(6): X is any amino acid
  MISC_FEATURE (7)..(7): X is Dha or Dhb
  MISC_FEATURE (9)..(9): X is Dha or Dhb
  MISC_FEATURE (10)..(12): X is any amino acid
AACXXXXPXXXXC

SEQ ID NO: 108 - protein, 13 aa, Artificial Sequence
  Peptide
AACKGPSPTPNNC

SEQ ID NO: 109 - protein, 13 aa, Artificial Sequence
  Peptide
AACSRSSPSRTYC

SEQ ID NO: 110 - protein, 13 aa, Artificial Sequence
  Peptide
AACTNSSPTQVTC

SEQ ID NO: 111 - protein, 13 aa, Artificial Sequence
  Peptide
AACNDASPSPNMC

SEQ ID NO: 112 - protein, 13 aa, Artificial Sequence
  Peptide
AACKLRTPSNQQC

SEQ ID NO: 113 - protein, 13 aa, Artificial Sequence
  Peptide
AACHIGSPSINQC

SEQ ID NO: 114 - protein, 13 aa, Artificial Sequence
  Peptide
AACGPTTPTSPPC

SEQ ID NO: 115 - protein, 13 aa, Artificial Sequence
  Peptide
AACPGPTPTNMPC

SEQ ID NO: 116 - protein, 13 aa, Artificial Sequence
  Peptide
AACMYLTPTRSLC

SEQ ID NO: 117 - protein, 13 aa, Artificial Sequence
  Peptide
AACTIFTPSPFFC

SEQ ID NO: 118 - protein, 13 aa, Artificial Sequence
  Peptide
AACTAPSPSVTQC

SEQ ID NO: 119 - protein, 13 aa, Artificial Sequence
  Peptide
AACLKPTPTQSTC

SEQ ID NO: 120 - protein, 13 aa, Artificial Sequence
  Peptide
AACSKGSPSPSHC

SEQ ID NO: 121 - protein, 13 aa, Artificial Sequence
  Peptide
AACTSHTPTHKNC

SEQ ID NO: 122 - protein, 13 aa, Artificial Sequence
  Peptide
AACVQTTPTLIGC

SEQ ID NO: 123 - protein, 13 aa, Artificial Sequence
  Peptide
AACPVSTPTLTSC

SEQ ID NO: 124 - protein, 13 aa, Artificial Sequence
  Peptide
AACSFQSPSLTHC

SEQ ID NO: 125 - protein, 13 aa, Artificial Sequence
  Peptide
AACSNTTPTPDYC

SEQ ID NO: 126 - protein, 13 aa, Artificial Sequence
  Peptide
AACQSNTPTRTPC

SEQ ID NO: 127 - protein, 13 aa, Artificial Sequence
  Peptide
AACLSTSPSKIPC

SEQ ID NO: 128 - protein, 13 aa, Artificial Sequence
  Peptide
AACTMATPSKNGC

SEQ ID NO: 129 - protein, 13 aa, Artificial Sequence
  Peptide
AACQPNSPSKKIC

SEQ ID NO: 130 - protein, 13 aa, Artificial Sequence
  Peptide
AACMPWTPSLFRC

SEQ ID NO: 131 - protein, 13 aa, Artificial Sequence
  Peptide
AACTFLSPSTPIC

SEQ ID NO: 132 - protein, 13 aa, Artificial Sequence
  Peptide
AACTSNTPTTRTC

SEQ ID NO: 133 - protein, 13 aa, Artificial Sequence
  Peptide
AACTIGSPSLRHC

SEQ ID NO: 134 - protein, 13 aa, Artificial Sequence
  Peptide
AACLFRTPTPFSC

SEQ ID NO: 135 - protein, 13 aa, Artificial Sequence
  Peptide
AACKDMTPSPKTC

SEQ ID NO: 136 - protein, 13 aa, Artificial Sequence
  Peptide
AACKLGTPTDKTC

SEQ ID NO: 137 - protein, 13 aa, Artificial Sequence
  Peptide
AACMDTSPTPPSC

SEQ ID NO: 138 - protein, 13 aa, Artificial Sequence
  Peptide
AACAFWSPSIMSC

SEQ ID NO: 139 - protein, 13 aa, Artificial Sequence
  Peptide
AACPPVSPSNDRC

SEQ ID NO: 140 - protein, 13 aa, Artificial Sequence
  Peptide
AACTMGTPTQSGC

SEQ ID NO: 141 - protein, 13 aa, Artificial Sequence
  Peptide
AACTNLSPTQDQC

SEQ ID NO: 142 - protein, 13 aa, Artificial Sequence
  Peptide
AACHKSSPTQVYC

SEQ ID NO: 143 - protein, 13 aa, Artificial Sequence
  Peptide
AACFIRTPSFQRC

SEQ ID NO: 144 - protein, 13 aa, Artificial Sequence
  Peptide
AACPKPTPSALKC

SEQ ID NO: 145 - protein, 13 aa, Artificial Sequence
  Peptide
AACLFRSPTPFSC

SEQ ID NO: 146 - protein, 13 aa, Artificial Sequence
  Peptide
AACHTPTPTTLSC

SEQ ID NO: 147 - protein, 13 aa, Artificial Sequence
  Peptide
AACSSRTPTTTEC

SEQ ID NO: 148 - protein, 13 aa, Artificial Sequence
  Peptide
AACNSSTPTPDTC

SEQ ID NO: 149 - protein, 13 aa, Artificial Sequence
  Peptide
AACNYWSPTYEDC

SEQ ID NO: 150 - protein, 13 aa, Artificial Sequence
  Peptide
AACKGASPSLPKC

SEQ ID NO: 151 - protein, 19 aa, Artificial Sequence
  Enzyme-Leader-Fusion
KPTTVKPPSDFEEWTINVC

SEQ ID NO: 152 - protein, 64 aa, Artificial Sequence; identical to SEQ ID NO: 37
  Leader

SEQ ID NO: 153 - protein, 1068 aa, Prochlorococcus marinus; identical to SEQ ID NO: 1

SEQ ID NO: 154 - protein, 1142 aa, Artificial Sequence
  Enzyme-Leader-Fusion
MSEEQLKAFIAKVQADTSLQEQLKAEGADVVAIAKAAGFTITTEDLNSHRQNLTDDELEGVAGGSASGSGSGSAMESPSSWKTSWLAAIAPDEPHKFDRRLEWDELSEENFFAALNSEPASLEEDDPCFEEALQDALEALKAAWDLPLLPVDNNLNRPFVDVWWPIRCHSAESLRQSFVSDSAGLADEIFDQLADSLLDRLCALGDQVLWEAFNKERTPGTMLLAHLGAAGDGSGPPVREHYERFIQSHRRNGLAPLLKEFPVLGRLIGTVLSLWFQGSVEMLQRICADRTVLQQCFAIPCGHHLKTVKQGLSDPHRGGRAVAVLEFADPNSTANSSMHVVYKPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAEVKQMQHLDIPFFTHLIDADALQLGGLEQELPGFIQTSGLAAAYERLRNLDTDEIAFQLRLIRGAVEARELHTTPESSPTLPPPATPEALMSSSAETSLEAAKRIAHRLLELAIRDSQGQVEWLGMDLGADGESFSFGPVGLSLYGGSIGIAHLLQRLQAQQVSLMDADAIQTAILQPLVGLVDQPSDDGRRRWWRDQPLGLSGCGGTLLALTLQGEQAMANSLLAAALPRFIEADQQLDLIGGCAGLIGSLVQLGTESALQLALRAGDHLIAQQNEEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCCGSLGLMVLLEMLSAGPWPIDNQLRSHCQDVAFQYRLQALQRCSAEPIKLRCFGTKEGLLVLPGFFTGLSGMGLALLEDDPSRAVVSQLISAGLWPTE

SEQ ID NO: 155 - protein, 1154 aa, Artificial Sequence
  Enzyme-Leader-Fusion
MSEEQLKAFIAKVQADTSLQEQLKAEGADVVAIAKAAGFTITTEDLNSHRQNLTDDELEGVAGGSGSGSGSGSGSGSGSGSGSGSAMESPSSWKTSWLAAIAPDEPHKFDRRLEWDELSEENFFAALNSEPASLEEDDPCFEEALQDALEALKAAWDLPLLPVDNNLNRPFVDVWWPIRCHSAESLRQSFVSDSAGLADEIFDQLADSLLDRLCALGDQVLWEAFNKERTPGTMLLAHLGAAGDGSGPPVREHYERFIQSHRRNGLAPLLKEFPVLGRLIGTVLSLWFQGSVEMLQRICADRTVLQQCFAIPCGHHLKTVKQGLSDPHRGGRAVAVLEFADPNSTANSSMHVVYKPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAEVKQMQHLDIPFFTHLIDADALQLGGLEQELPGFIQTSGLAAAYERLRNLDTDEIAFQLRLIRGAVEARELHTTPESSPTLPPPATPEALMSSSAETSLEAAKRIAHRLLELAIRDSQGQVEWLGMDLGADGESFSFGPVGLSLYGGSIGIAHLLQRLQAQQVSLMDADAIQTAILQPLVGLVDQPSDDGRRRWWRDQPLGLSGCGGTLLALTLQGEQAMANSLLAAALPRFIEADQQLDLIGGCAGLIGSLVQLGTESALQLALRAGDHLIAQQNEEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCCGSLGLMVLLEMLSAGPWPIDNQLRSHCQDVAFQYRLQALQRCSAEPIKLRCFGTKEGLLVLPGFFTGLSGMGLALLEDDPSRAVVSQLISAGLWPTE

SEQ ID NO: 156 - protein, 1164 aa, Artificial Sequence
  Enzyme-Leader-Fusion
MSEEQLKAFIAKVQADTSLQEQLKAEGADVVAIAKAAGFTITTEDLNSHRQNLTDDELEGVAGGSGSGSGSGSGSGSGSGSGSGSGSGSGSGSGSAMESPSSWKTSWLAAIAPDEPHKFDRRLEWDELSEENFFAALNSEPASLEEDDPCFEEALQDALEALKAAWDLPLLPVDNNLNRPFVDVWWPIRCHSAESLRQSFVSDSAGLADEIFDQLADSLLDRLCALGDQVLWEAFNKERTPGTMLLAHLGAAGDGSGPPVREHYERFIQSHRRNGLAPLLKEFPVLGRLIGTVLSLWFQGSVEMLQRICADRTVLQQCFAIPCGHHLKTVKQGLSDPHRGGRAVAVLEFADPNSTANSSMHVVYKPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAEVKQMQHLDIPFFTHLIDADALQLGGLEQELPGFIQTSGLAAAYERLRNLDTDEIAFQLRLIRGAVEARELHTTPESSPTLPPPATPEALMSSSAETSLEAAKRIAHRLLELAIRDSQGQVEWLGMDLGADGESFSFGPVGLSLYGGSIGIAHLLQRLQAQQVSLMDADAIQTAILQPLVGLVDQPSDDGRRRWWRDQPLGLSGCGGTLLALTLQGEQAMANSLLAAALPRFIEADQQLDLIGGCAGLIGSLVQLGTESALQLALRAGDHLIAQQNEEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCCGSLGLMVLLEMLSAGPWPIDNQLRSHCQDVAFQYRLQALQRCSAEPIKLRCFGTKEGLLVLPGFFTGLSGMGLALLEDDPSRAVVSQLISAGLWPTE

SEQ ID NO: 157 - protein, 1216 aa, Artificial Sequence
  Enzyme-Leader-Fusion
MSEEQLKAFIAKVQADTSLQEQLKAEGADVVAIAKAAGFTITTEDLNSHRQNLTDDELEGVAGGSASGSGSGSAMESPSSWKTSWLAAIAPDEPHKFDRRLEWDELSEENFFAALNSEPASLEEDDPCFEEALQDALEALKAAWDLPLLPVDNNLNRPFVDVWWPIRCHSAESLRQSFVSDSAGLADEIFDQLADSLLDRLCALGDQVLWEAFNKERTPGTMLLAHLGAAGDGSGPPVREHYERFIQSHRRNGLAPLLKEFPVLGRLIGTVLSLWFQGSVEMLQRICADRTVLQQCFAIPCGHHLKTVKQGLSDPHRGGRAVAVLEFADPNSTANSSMHVVYKPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAEVKQMQHLDIPFFTHLIDADALQLGGLEQELPGFIQTSGLAAAYERLRNLDTDEIAFQLRLIRGAVEARELHTTPESSPTLPPPATPEALMSSSAETSLEAAKRIAHRLLELAIRDSQGQVEWLGMDLGADGESFSFGPVGLSLYGGSIGIAHLLQRLQAQQVSLMDADAIQTAILQPLVGLVDQPSDDGRRRWWRDQPLGLSGCGGTLLALTLQGEQAMANSLLAAALPRFIEADQQLDLIGGCAGLIGSLVQLGTESALQLALRAGDHLIAQQNEEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCCGSLGLMVLLEMLSAGPWPIDNQLRSHCQDVAFQYRLQALQRCSAEPIKLRCFGTKEGLLVLPGFFTGLSGMGLALLEDDPSRAVVSQLISAGLWPTESASGSGSGSAMSEEQLKAFIAKVQADTSLQEQLKAEGADVVAIAKAAGFTITTEDLNSHRQNLTDDELEGVAGG

SEQ ID NO: 158 - protein, 1226 aa, Artificial Sequence
  Enzyme-Leader-Fusion
MSEEQLKAFIAKVQADTSLQEQLKAEGADVVAIAKAAGFTITTEDLNSHRQNLTDDELEGVAGGSASGSGSGSAMESPSSWKTSWLAAIAPDEPHKFDRRLEWDELSEENFFAALNSEPASLEEDDPCFEEALQDALEALKAAWDLPLLPVDNNLNRPFVDVWWPIRCHSAESLRQSFVSDSAGLADEIFDQLADSLLDRLCALGDQVLWEAFNKERTPGTMLLAHLGAAGDGSGPPVREHYERFIQSHRRNGLAPLLKEFPVLGRLIGTVLSLWFQGSVEMLQRICADRTVLQQCFAIPCGHHLKTVKQGLSDPHRGGRAVAVLEFADPNSTANSSMHVVYKPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAEVKQMQHLDIPFFTHLIDADALQLGGLEQELPGFIQTSGLAAAYERLRNLDTDEIAFQLRLIRGAVEARELHTTPESSPTLPPPATPEALMSSSAETSLEAAKRIAHRLLELAIRDSQGQVEWLGMDLGADGESFSFGPVGLSLYGGSIGIAHLLQRLQAQQVSLMDADAIQTAILQPLVGLVDQPSDDGRRRWWRDQPLGLSGCGGTLLALTLQGEQAMANSLLAAALPRFIEADQQLDLIGGCAGLIGSLVQLGTESALQLALRAGDHLIAQQNEEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCCGSLGLMVLLEMLSAGPWPIDNQLRSHCQDVAFQYRLQALQRCSAEPIKLRCFGTKEGLLVLPGFFTGLSGMGLALLEDDPSRAVVSQLISAGLWPTESASGSGSGSGSTSGSGSGSAMSEEQLKAFIAKVQADTSLQEQLKAEGADVVAIAKAAGFTITTEDLNSHRQNLTDDELEGVAGG

SEQ ID NO: 159 - protein, 1236 aa, Artificial Sequence
  Enzyme-Leader-Fusion
MSEEQLKAFIAKVQADTSLQEQLKAEGADVVAIAKAAGFTITTEDLNSHRQNLTDDELEGVAGGSASGSGSGSAMESPSSWKTSWLAAIAPDEPHKFDRRLEWDELSEENFFAALNSEPASLEEDDPCFEEALQDALEALKAAWDLPLLPVDNNLNRPFVDVWWPIRCHSAESLRQSFVSDSAGLADEIFDQLADSLLDRLCALGDQVLWEAFNKERTPGTMLLAHLGAAGDGSGPPVREHYERFIQSHRRNGLAPLLKEFPVLGRLIGTVLSLWFQGSVEMLQRICADRTVLQQCFAIPCGHHLKTVKQGLSDPHRGGRAVAVLEFADPNSTANSSMHVVYKPKDMAVDAAYQATLADLNTHSDLSPLRTLAIHNGNGYGYMEHVVHHLCANDKELTNFYFNAGRLTALLHLLGCTDCHHENLIACGDQLLLIDTETLLEADLPDHISDASSTTAQPKPSSLQKQFQRSVLRSGLLPQWMFLGESKLAIDISALGMSPPNKPERIALGWLGFNSDGMMPGRVSQPVEIPTSLPVGIGEVNPFDRFLEDFCDGFSMQSEALIKLRNRWLDVNGVLAHFAGLPRRIVLRATRVYFTIQRQQLEPTALRSPLAQALKLEQLTRSFLLAESKPLHWPIFAAEVKQMQHLDIPFFTHLIDADALQLGGLEQELPGFIQTSGLAAAYERLRNLDTDEIAFQLRLIRGAVEARELHTTPESSPTLPPPATPEALMSSSAETSLEAAKRIAHRLLELAIRDSQGQVEWLGMDLGADGESFSFGPVGLSLYGGSIGIAHLLQRLQAQQVSLMDADAIQTAILQPLVGLVDQPSDDGRRRWWRDQPLGLSGCGGTLLALTLQGEQAMANSLLAAALPRFIEADQQLDLIGGCAGLIGSLVQLGTESALQLALRAGDHLIAQQNEEGAWSSSSSQPGLLGFSHGTAGYAAALAHLHAFSADERYRTAAAAALAYERARFNKDAGNWPDYRSIGRDSDSDEPSFMASWCHGAPGIALGRACLWGTALWDEECTKEIGIGLQTTAAVSSVSTDHLCCGSLGLMVLLEMLSAGPWPIDNQLRSHCQDVAFQYRLQALQRCSAEPIKLRCFGTKEGLLVLPGFFTGLSGMGLALLEDDPSRAVVSQLISAGLWPTESASGSGSGSGSGSGSGSGSGSTSGSGSGSAMSEEQLKAFIAKVQADTSLQEQLKAEGADVVAIAKAAGFTITTEDLNSHRQNLTDDELEGVAGG

SEQ ID NO: 160 - protein, 34 aa, Planktothrix agardhii; identical to SEQ ID NO: 76

SEQ ID NO: 161 - protein, 374 aa, Artificial Sequence
  Enzyme-Leader-Fusion
MSKNVKVSAPKAVPFFARFLAEQAVEANNSNSAPSASGSGSGSAMTVLIFTFSNDNESIPLVVKAIEAKGEKAFRFDTDRFPTEVKLDIYSGDFEGGIVTDGEHKLDLSEVSSVWYRRMRYGAKLPDSMDQQFREASLKECRLTVRGLIASLPGFHFDPMINVDRANNKQLQLQIARQLGLLTPRTLTSNNPEAVKQFAQDCKEQGIVTKMLSQFAIYGENKEENVVFTSPVTDDDLDNLEGLSFCPMTFQENVPKALELRTIIVGKQVFTAAINSQQLDGATYDWRKEGRALHEKWQAYNLPEDIEKKLLQLTAYFGLNYGAIDIIVTPDGRHVFLEINPVGEFFWLELFAPYFPISQAIAEILLTPTNQKPF

SEQ ID NO: 162 - protein, 384 aa, Artificial Sequence
  Enzyme-Leader-Fusion
MSKNVKVSAPKAVPFFARFLAEQAVEANNSNSAPSASGSGSGSGSTSGSGSGSAMTVLIFTFSNDNESIPLVVKAIEAKGEKAFRFDTDRFPTEVKLDIYSGDFEGGIVTDGEHKLDLSEVSSVWYRRMRYGAKLPDSMDQQFREASLKECRLTVRGLIASLPGFHFDPMINVDRANNKQLQLQIARQLGLLTPRTLTSNNPEAVKQFAQDCKEQGIVTKMLSQFAIYGENKEENVVFTSPVTDDDLDNLEGLSFCPMTFQENVPKALELRTIIVGKQVFTAAINSQQLDGATYDWRKEGRALHEKWQAYNLPEDIEKKLLQLTAYFGLNYGAIDIIVTPDGRHVFLEINPVGEFFWLELFAPYFPISQAIAEILLTPTNQKPF

SEQ ID NO: 163 - protein, 394 aa, Artificial Sequence
  Enzyme-Leader-Fusion
MSKNVKVSAPKAVPFFARFLAEQAVEANNSNSAPSASGSGSGSGSGSGSGSGSGSTSGSGSGSAMTVLIFTFSNDNESIPLVVKAIEAKGEKAFRFDTDRFPTEVKLDIYSGDFEGGIVTDGEHKLDLSEVSSVWYRRMRYGAKLPDSMDQQFREASLKECRLTVRGLIASLPGFHFDPMINVDRANNKQLQLQIARQLGLLTPRTLTSNNPEAVKQFAQDCKEQGIVTKMLSQFAIYGENKEENVVFTSPVTDDDLDNLEGLSFCPMTFQENVPKALELRTIIVGKQVFTAAINSQQLDGATYDWRKEGRALHEKWQAYNLPEDIEKKLLQLTAYFGLNYGAIDIIVTPDGRHVFLEINPVGEFFWLELFAPYFPISQAIAEILLTPTNQKPF

SEQ ID NO: 164 - protein, 378 aa, Artificial Sequence
  Enzyme-Leader-Fusion
MTVLIFTFSNDNESIPLVVKAIEAKGEKAFRFDTDRFPTEVKLDIYSGDFEGGIVTDGEHKLDLSEVSSVWYRRMRYGAKLPDSMDQQFREASLKECRLTVRGLIASLPGFHFDPMINVDRANNKQLQLQIARQLGLLTPRTLTSNNPEAVKQFAQDCKEQGIVTKMLSQFAIYGENKEENVVFTSPVTDDDLDNLEGLSFCPMTFQENVPKALELRTIIVGKQVFTAAINSQQLDGATYDWRKEGRALHEKWQAYNLPEDIEKKLLQLTAYFGLNYGAIDIIVTPDGRHVFLEINPVGEFFWLELFAPYFPISQAIAEILLTPTNQKPFSASGSGSGSAMSKNVKVSAPKAVPFFARFLAEQAVEANNSNSAPEFAL

SEQ ID NO: 165 - protein, 388 aa, Artificial Sequence
  Enzyme-Leader-Fusion
MTVLIFTFSNDNESIPLVVKAIEAKGEKAFRFDTDRFPTEVKLDIYSGDFEGGIVTDGEHKLDLSEVSSVWYRRMRYGAKLPDSMDQQFREASLKECRLTVRGLIASLPGFHFDPMINVDRANNKQLQLQIARQLGLLTPRTLTSNNPEAVKQFAQDCKEQGIVTKMLSQFAIYGENKEENVVFTSPVTDDDLDNLEGLSFCPMTFQENVPKALELRTIIVGKQVFTAAINSQQLDGATYDWRKEGRALHEKWQAYNLPEDIEKKLLQLTAYFGLNYGAIDIIVTPDGRHVFLEINPVGEFFWLELFAPYFPISQAIAEILLTPTNQKPFSASGSGSGSGSTSGSGSGSAMSKNVKVSAPKAVPFFARFLAEQAVEANNSNSAPEFAL

SEQ ID NO: 166 - protein, 398 aa, Artificial Sequence
  Enzyme-Leader-Fusion
MTVLIFTFSNDNESIPLVVKAIEAKGEKAFRFDTDRFPTEVKLDIYSGDFEGGIVTDGEHKLDLSEVSSVWYRRMRYGAKLPDSMDQQFREASLKECRLTVRGLIASLPGFHFDPMINVDRANNKQLQLQIARQLGLLTPRTLTSNNPEAVKQFAQDCKEQGIVTKMLSQFAIYGENKEENVVFTSPVTDDDLDNLEGLSFCPMTFQENVPKALELRTIIVGKQVFTAAINSQQLDGATYDWRKEGRALHEKWQAYNLPEDIEKKLLQLTAYFGLNYGAIDIIVTPDGRHVFLEINPVGEFFWLELFAPYFPISQAIAEILLTPTNQKPFSASGSGSGSGSGSGSGSGSGSTSGSGSGSAMSKNVKVSAPKAVPFFARFLAEQAVEANNSNSAPEFAL

SEQ ID NO: 167 - protein, 10 aa, Artificial Sequence
  Linker
SASGSGSGSA

SEQ ID NO: 168 - protein, 22 aa, Artificial Sequence
  Linker
SGSGSGSGSGSGSGSGSGSGSA

SEQ ID NO: 169 - protein, 32 aa, Artificial Sequence
  Linker
SGSGSGSGSGSGSGSGSGSGSGSGSGSGSGSA

SEQ ID NO: 170 - protein, 20 aa, Artificial Sequence
  Linker
SASGSGSGSGSTSGSGSGSA

SEQ ID NO: 171 - protein, 30 aa, Artificial Sequence
  Linker
SASGSGSGSGSGSGSGSGSGSTSGSGSGSA

(19) United States
(12) Patent Application Publication
(10) Pub. No.: US 2017/0240883 A1
(43) Pub. Date: Aug. 24, 2017

(54) CYCLIC PEPTIDES EXPRESSED BY A GENETIC PACKAGE

(71) Applicant: Miti Biosystems GmbH, Wien (AT)

(72) Inventors: Guntram CHRISTIANSEN, Vienna (AT); WALKER Edward, Melbourn (GB)

(21) Appl. No.: 15/501,823

(22) PCT Filed: Aug. 11, 2015
(86) PCT No.: PCT/EP2015/068442

§ 371 Date: Feb. 3, 2017

(30) Foreign Application Priority Data
Aug. 11, 2014 (EP) 14180574.7
Aug. 11, 2014 (EP) 14180575.4

(51) Int. Cl. C12N 15/10; C07K 14/405

(52) U.S. Cl. CPC C12N 15/1037; C07K 14/405; C07K 2319/00

(57) ABSTRACT

The invention provides a method of biosynthesis of a cyclic peptide by enzymatically transforming a substrate peptide into the cyclic peptide, wherein the substrate peptide is expressed by a displaying genetic package and comprises at least one Ser or Thr residue and at least one Cys residue, and the enzyme is a post-translationally modifying enzyme (PTME) which is a bifunctional thioether bridge forming dehydratase and cyclase, thereby obtaining the displaying genetic package carrying the cyclic peptide comprising a thioether bridge crosslinking the at least one Ser or Thr to Cys; and further a library of immobilised cyclic peptides, each with a length of at least 10 amino acids, comprising a variety of at least 10⁶ library members, which variety comprises at least one of a) a different position of the thioether bridge forming a loop within the substrate peptide; or b) a different number of loops within the substrate peptide.

The invention relates to the biosynthesis of a cyclic peptide by enzymatically transforming a substrate peptide which is encoded by a displaying genetic package, into the cyclic peptide which is expressed by the genetic package, using a post-translationally modifying enzyme (PTME), and a library of cyclic peptides expressed by the genetic package.

### BACKGROUND

The search for new compounds in many cases makes use of large libraries of compounds to screen and identify a compound that has a desired activity or characteristic. Combinatorial peptide library technology is a valuable resource for drug discovery and development. Recombinant peptide libraries displayed on phage or other viral particles have proven especially useful in such screens. Numerous groups are working to develop biologically active peptides obtained from peptide libraries in the search of novel treatments for many human diseases and illnesses.

Phage-displayed peptide library technology has been widely used in the search of novel treatments for many human diseases and illnesses. Peptide libraries displayed on filamentous phage have been used as a screening resource for identifying peptides bound to any given target thereby showing pharmacologic effects. Peptides so identified can subsequently be synthesized in bulk using conventional synthetic chemistry methods.

The bacteriophage M13 is a non-enveloped filamentous Escherichia coli phage of the Inoviridae family with a single stranded (ss)DNA genome. The nucleocapsid consists of four bacteriophage proteins with different copy numbers: pVIII approximately 2700 copies while pIII, pVI, pVII and pIX are present with up to 5 copies. Among other, bacteriophages like T4, T7, fd and lambda, M13 has been successfully used for phage display for use in a biotechnological screening method. In such a screening approach a random library of peptides is presented on the surface of the nucleocapsid of the phage M13 to study interaction of the different phages with a binding partner (protein-protein, protein-DNA, etc). Usually synthetic oligonucleotides are cloned into genes coding for proteins which constitute the nucleocapsid and thereby the peptide of interest (or a library of different peptides) is presented on the surface of the phage M13 nucleocapsid for subsequent binding studies.

The phage display methods typically involve the insertion of random oligonucleotides into a phage genome such that they direct a bacterial host to express peptide libraries fused to phage coat proteins (e. g., filamentous phage pIII, pVI or pVIII). The advantages of this technique are in the small dimension of the phage allowing to handle libraries with 10¹² different individuals and in the physical linkage of the displayed peptides with the genetic information that encode them.

The basic phage display technology has been expanded to include peptide libraries that are displayed from genetic packages other than phage, such as eukaryotic viruses, bacteria and yeast cells, but also by in vitro display technologies, e.g. employing ribosome-display, RNA/DNA-peptide fusion molecules, immobilised peptide display, emulsion compartmentalization and display, plasmid display, covalent display, solid phase display, microarray display and the like.

The principles and strategy are closely analogous to those employed for phage, namely, that nucleic acids encoding peptides to be displayed are inserted into the genome of the genetic package to create and express the peptides to be screened. Expression of peptides immobilised by the genetic package results in display of peptides, e.g. at a cell or viral surface.

In an effort to increase diversity of a library though a secondary peptide structure some groups have produced conformationally-constrained peptides through chemical or enzymatic reactions.

Bosma et al (Applied and Environmental Microbiology 2011; 77(19) 6794-6801) describe the bacterial display and screening of posttranslationally thioether-stabilized peptides. The peptides are posttranslationally modified by thioether-bridge installing enzymes in Lactococcus lactis, followed by export and sortase-mediated covalent coupling to the lactococcal cell wall.

Lanthipeptide synthetases are enzymes known to produce ribosomally synthesised and posttranslationally modified peptides (also called RiPPs), i.e. lanthionine-containing peptides. Zhang et al. (PNAS 2012; 109(45) 18361-18366) describe the biosynthesis of lanthiopeptides and the four classes of synthetases: class I (LanB and LanC), class II (LanM), class III (LanKC), and class IV (LanL).

ProcM is known as a substrate-tolerant lanthipeptide synthetase produced by marine cyanobacteria of the genus Prochlorococcus. ProcM and its mode of action is described by Mukgherjee et al. (J. Am. Chem. Soc. 2014; 136 (29), pp 10450-10459). It dehydrates core peptides containing a variety of sequences with different residues flanking Ser and Thr and catalyzes cyclisation by forming thioether rings with Cys residues located on either side of the dehydrated residues. Thereby 29 different Prochlorosins with diverse ring topologies are produced by this ProcM.

The class II lanthipeptide synthetase ProcM is further described by Thibodeaux et al. (Chem. Commun. 2012; 48(86) 10615-10617). Rationally engineered ProcM mutants were described to phosphorylate Ser and Thr residues located in a variety of peptide sequences.

The substrate specificity of a lanthipeptide synthetase or other posttranslationally modifying enzymes is mainly determined by the presence of a leader peptide which is positioned at the N-terminus or adjacent to a core peptide to be modified. The leader acts as a recognition motif and optionally as a translocation motif to express the posttranslationally modified peptide.

WO2012/005578 A1 describes a fusion peptide comprising an N-terminal lantibiotic leader sequence and a core sequence to be post-translationally modified to a dehydroresidue or thioether-bridge containing polypeptide.

Oman et al. (J. Am. Chem. Soc. 2012; 134: 6952-6955) describe the engineered lantibiotic synthetase LctM, wherein a hexa-His-tagged LctA leader peptide is fused to the N-terminus of the LctM synthetase via a Gly/Ser linker sequence. The fusion enzyme was expressed in E. coli. Incubation of the core peptide of LctA obtained by solid-phase peptide synthesis with the fusion enzyme resulted in a dehydrated core peptide. Furthermore, thioether rings were formed in the dehydrated product.

WO2012/019928 A1 discloses the replicable genetic package displaying a peptide having at least one intramolecular cyclic bond between two heteroatoms of amino acid side chains. For example, a peptide and a leader is co-expressed and the peptide is posttranslationally modified. The modified peptides are displayed by an in vitro or biological display system.

### SUMMARY OF THE INVENTION

It is the objective of the present invention to provide for an increased variety of cyclic peptides to enable the selection of suitable active substances, e.g. for pharmaceutical, analytical, agricultural, pesticidal or industrial use.

The object is solved by the claimed subject matter.

According to the invention there is provided a method of biosynthesis of a cyclic peptide by enzymatic transforming a substrate peptide into the cyclic peptide, wherein the substrate peptide is expressed by a displaying genetic package and comprises at least one Ser or Thr residue and at least one Cys residue, and the enzyme is a post-translationally modifying enzyme (PTME) which is a bifunctional thioether bridge forming dehydratase and cyclase, thereby obtaining the displaying genetic package carrying the cyclic peptide comprising a thioether bridge crosslinking the at least one Ser or Thr to Cys. Specifically, the substrate peptide is a peptide to be modified for introducing an intramolecular cycle (loop), e.g. a linear substrate peptide.

Specifically, the PTME is originating from a cyanobacterium, preferably the genus Prochlorococcus or Synechococcus, which is preferably comprising

a) the catalytic sites of a ProcM enzyme which is any of ProcM9313 identified by the amino acid sequence SEQ ID 1, ProcM9303 identified by the amino acid sequence SEQ ID 2, and ProcM9916 identified by the amino acid sequence SEQ ID 3; or

b) the amino acid sequence SEQ ID 1, SEQ ID 2 or SEQ ID 3, or an amino acid sequence with at least 60% sequence identity to any of SEQ ID 1, SEQ ID 2, or SEQ ID 3; or

c) a functionally active variant of any of the foregoing.

More specifically,
the functionally active variant of embodiment a) comprises at least one modified catalytic site which is the dehydratase or the cyclase acting region and preferably comprises at least one dehydratase acting region and at least one cyclase acting region, wherein the dehydratase acting region is selected from the group consisting of the amino acid sequence SEQ ID 4, 6, and 8, and wherein the cyclase acting region is selected from the group consisting of the amino acid sequence SEQ ID 5, 7, and 9, and which is characterized by at least one point mutation and at least 60% sequence identity to any of SEQ ID 3 to SEQ ID 9; or
the functionally active variant of embodiment b) comprises at least one point mutation and/or is a size variant;
and wherein the functionally active variant is capable of dehydrating and cycling amino acid residues of the substrate peptide.

Preferably, the PTME comprises the combination of the dehydratase acting region and at least one cyclase acting region, which is the combination of SEQ ID 4 and 5, or 6 and 7, or 8 and 9, wherein in such combination one or both of the acting regions are naturally-occurring or functionally active variants.

Specifically, the PTME is ProcM selected from the group consisting of ProcM9313, ProcM9303, and ProcM9916.

ProcM9313 originating from Prochlorococcus marinus is specifically characterized by its amino acid sequence which comprises or consists of SEQ ID 1 and has bifunctional activity determined by a dehydratase active site comprising SEQ ID 4 and further by the cyclase active site comprising SEQ ID 5.

ProcM9303 originating from Prochlorococcus marinus is specifically characterized by its amino acid sequence which comprises or consists of SEQ ID 2 and has bifunctional activity determined by a dehydratase active site comprising SEQ ID 6 and further by the cyclase active site comprising SEQ ID 7.

ProcM9916 originating from Synechococcus sp. is specifically characterized by its amino acid sequence which comprises or consists of SEQ ID 3 and has bifunctional activity determined by a dehydratase active site comprising SEQ ID 8 and further by the cyclase active site comprising SEQ ID 9.

According to a specific aspect, the substrate peptide comprises the sequence Ser/Thr-Xaa₍ₙ₎-Cys or Cys-Xaa₍ₙ₎-Ser/Thr, wherein n=0-100 amino acids.

Specifically, the thioether bridge is formed between side-chains of the Ser or Thr to Cys, thereby forming a loop of 2-102 amino acids.

According to a specific aspect, the cyclic peptide is displayed on the surface of the genetic package, preferably wherein the cyclic peptide is part of a surface display system comprising the nucleic acid sequence encoding the substrate peptide.

Specifically, the genetic package is selected from the group consisting of a bacteriophage, virus, bacterium, yeast, and ribosome, or which comprises a RNA/DNA-peptide fusion molecule.

The display by the genetic package is suitably a biological (or in vivo) display system, e.g. employing a replicable genetic package such as a cell or virus and in particular a bacteriophage, or an in vitro display system.

Specifically, the genetic package is a filamentous phage, preferably a bacteriophage, such as M13, and the cyclic peptide is immobilised onto the bacteriophage by fusion to a coat protein, preferably selected from the group consisting of gene III, gene VI, gene VII, gene VIII, and gene IX.

Specifically, the cyclic peptide is bound to the surface of the genetic package via a peptide linker and/or a disulfide bridge. In particular, the cyclic peptide can be immobilised onto the surface of a phage by Cys-display.

According to a specific aspect, the substrate peptide is expressed and transformed into the cyclic peptide in the inner compartment of the genetic package, e.g. in the inner part or the cytosol of a cell, and the transformed, i.e. the dehydrated and/or cycled peptide, is transported to the outer compartment of the genetic package, e.g. presented at the cell wall or the coat of a virus/phage.

Specifically, the substrate peptide is transformed in the soluble form, or in the immobilised form, such as upon display by the genetic package.

According to a specific aspect, a repertoire of variant substrate peptides is transformed in a one-step process, thereby producing a library of cyclic peptides.

Specifically, a library of substrate peptides are provided as a library of nucleic acid molecules or a library of peptide sequences, and transformed by incubating with the PTME. The one step process may be carried out by incubating a series of the individual substrate peptides or library members with only one PTME preparation, e.g. in a consecutive, parallel or simultaneous way. Particular one-step processes are carried out in only one container, such that a mixture of individual substrate peptides or library members are transformed at once.

Further process steps may precede or follow the one-step enzymatic transformation with the PTME, such as further enzymatic processes, selection, recovery or purification steps.

Specifically, the method may comprise a further process step wherein the cyclic peptide is cleaved off the genetic package.

According to a specific aspect, the variant substrate peptides comprise a randomised peptide sequence to produce the repertoire.

Specifically, the substrate peptide is partly or fully randomised.

According to a specific embodiment, the nucleic acid encoding the substrate peptide is randomised, wherein the proportion of at least one base at a specified position in the codon is varied to bias the codon towards coding for a desired amino acid.

A specifically desired amino acid is any of a serine, threonine or cysteine. Thereby, the number of potential loops formed by a thioether bridge may be increased.

According to a specific aspect, the substrate peptide is transformed by the PTME in close proximity to a cognate PTME-leader peptide.

Specifically, the PTME-leader peptide is ligated to at least one of the substrate peptide or the PTME, preferably wherein the PTME-leader peptide is fused C- or N-terminally to the substrate peptide and/or the PTME, and/or incorporated in the substrate peptide or the PTME.

In particular, the PTME-leader peptide may be conjugated to the PTME by recombinant fusion, chemical conjugation, or affinity binding.

Specifically, the PTME is provided as an enzyme-leader-fusion protein (ELF, or Elf) wherein the PTME is fused to a cognate PTME-leader peptide.

Specifically, the ELF comprises the PTME-leader peptide C- or N-terminally fused to the PTME, optionally with a linker of 1-30 amino acids, or incorporated in the PTME, optionally flanked by one or more amino acids of an inserted expression construct.

Specifically, the linker is a linear peptidic linker composed of one or more amino acids selected from the group consisting of glycine, serine, alanine, and threonine. Specifically, the linker is composed of a series of one or more of a first amino acid combined with a series of one or more of a second amino acid, such as to obtain a combination of two different amino acids in any order, wherein the first amino acid is any of glycine, alanine, or threonine, and the second amino acid is serine. For example the linker contains or is composed of a combination of glycine and serine, or a combination of alanine and serine, or a combination of threonine and serine.

Specifically, the ELF is recombinantly produced, and optionally co-expressed by the displaying genetic package, or produced by organic synthesis.

Specifically, the ELF is a fusion protein produced by a recombinant host cell which comprises a nucleic acid encoding the ELF, or the PTME-leader peptide and the PTME within the same open reading frame.

Specifically, the PTME-leader peptide is a co-substrate recognized by the PTME, preferably which is naturally-occurring or a functionally active derivative thereof comprising one or more point mutations, e.g. with a sequence identity of at least 60%, preferably at least 70%, or at least 80%, or at least 90%.

According to a specific aspect, the PTME-leader peptide and the PTME are of the same or different origin, preferably wherein the PTME-leader peptide and/or the PTME are of a Prochlorococcus or Synechococcus origin, or a functionally active derivative thereof, or artificial.

Functionally active derivatives may e.g. be provided as non-naturally occurring mutations, or from analogous sequences obtained from different species within the same genus, or even from a different genus. Typically, functionally active derivatives of a PTME-leader peptide or the PTME have a sequence identity of at least 60% as compared to naturally occurring PTME-leader peptide and PTME sequences, preferably at least 70%, or at least 80%, or at least 90%. Those mutants which are not considered functionally active derivatives are considered as being artificial. In particular, randomised sequences with a sequence identity of less than 60% as compared to naturally occurring PTME-leader peptide and PTME sequences are typically considered as being artificial.

According to a specific aspect, at least two different PTME-leader peptides are used which are recognized by one or more PTME. Typically, a cognate PTME-leader peptide is employed for one or more PTME, or for each of the PTME.

Specifically, at least two different PTME are used to transform the substrate peptide into the cyclic peptide, which are of the same or different class, preferably wherein the class is selected from the group consisting of a bifunctional dehydratase-cyclase, dehydratase, cyclase, carboxylate-amine ligase, decarboxylase, epimerase, hydroxylase, peptidase, dehydratase, transferase, esterase, oxygenase, isomerase and transglutaminase (e.g. microbial transglutaminase, mTG).

Specifically, a set of at least one ELF and a further enzyme, which is a PTME, with or without being fused to the cognate PTME-leader, is provided, wherein the ELF and the further enzyme differ in the PTME-leader and/or the PTME.

Specifically, at least two different ELFs is provided, which differ in the PTME-leader and/or the PTME.

Specifically, a combination of at least two different PTME is used, wherein one is the bifunctional thioether bridge forming dehydratase and cyclase, such as ProcM, and the at least one further PTME is selected from any of the following (database accession numbers in brackets):

a) MvdB (ACC54548), MvdC (ACC54549), and MvdD (ACC54550);

b) LanM (AAU25567), LanB (AFE10374) and LanC (AGY89934)

b) SpaB (AAB91586) and SpaC (AAB91588);

c) NisB (CAA48381) and NisC (CAA48383)

d) PatD (AAY21153) and PatG (AAY21156);

e) TpdB (ACS83782), TpdC(ACS83783), TpdD(ACS83784), TpdE(ACS83785) and TpdG(ACS83787); TpdJ1(ACS83778) and TpdJ2(ACS83779)

f) McbB(CAT00698), McbC(CAT00697) and McbD(CAT00696);

g) McjB(AAD28495) and McjC(AAD28496);

h) TrnC(AED99784) and TrnD(AED99785);

i) PoyC(AFS60638), PoyD(AFS60640), PoyE(AFS60641), PoyF(AFS60642) and PoyI(AFS60645);

j) BmbA(CCM09441), BmbB(CCM09442), BmbD(CCM09445),

BmbE(CCM09446), BmbF(CCM09447), BmbG(CCM09448), BmbH(CCM09449), BmbI(CCM09450), BmbJ(CCM09451);

k) AlbA(NP_391617), AlbE(NP_391621) and AlbF(NP_391622);

l) MceC (AAL08396), MceD (AAL08397), MceI(AAL08402) and MceJ(AAL08403);

m) CypH(ADR72963) and CypL(ADR72964);

and optionally one or more further PTME.

According to a specific aspect, the PTME is produced by the displaying package or provided as a separate enzyme, in particular as an enzyme produced by a recombinant host. Specifically, the PTME may be provided as a recombinant molecule expressed by a recombinant host cell, e.g. co-expressed with the PTME-leader and/or the substrate peptide. Alternatively, the PTME may be produced by organic synthesis. For co-expression, the nucleic acids encoding the PTME and any of the PTME-leader and/or the substrate peptide may be incorporated or comprised in one or more expression vectors. Host cells comprising the respective nucleic acids and the expression vectors may be provided to produce the cyclic peptides in vivo.

Recombinant host cells may e.g. be cultivated to express the nucleic acid encoding the PTME with or without co-expressing the PTME-leader and/or the substrate peptide, e.g. to obtain the PTME or the ELF or the cycled peptide as an expression product, and harvesting the expression product from the host cell culture.

According to a specific embodiment, the cyclic peptide is a polycyclic peptide comprising at least two heteroatom bridges, wherein a heteroatom bridge is linking an amino acid side chain to another amino acid residue of the substrate peptide thereby forming a loop, preferably at least two thioether bridges.

Specifically, a heteroatom bridge is a covalent bond linking two atoms selected from the group consisting of C, N, O and S. This specifically includes C—N, C—O, C—S, N—N, N—O, N—S, O—O, O—S and S—S bonding, in an appropriate chemical sense including double bonds. Preferably, the cyclic bond is linking two different atoms selected from the group consisting of C, N, O and S.

Specifically, the polycyclic peptide comprises overlapping loops and/or loops within loops.

The invention further provides for a library of immobilised cyclic peptides, each with a length of at least 10 amino acids, or at least 11, at least 12, at least 13, at least 14, at least 15, at least 16, at least 17, at least 18, at least 19, or at least 20 amino acids, obtainable by a method of the invention, comprising a variety of at least 10⁶ library members, preferably at least 10⁷, or at least 10⁸, or at least 10⁹, or more e.g. up to 10¹² individual library members, which variety comprises at least one of

a) a different position of the thioether bridge forming a loop within the substrate peptide; or

b) a different number of loops within the substrate peptide.

Typically, the library is obtained by mutating or randomising a parent substrate peptide, and transforming the variants produced by mutation or randomisation into the variety of cyclic structures. While there are many different possibilities to randomise a substrate peptide, a variety of random cyclic structures are obtained.

Upon randomising a parent substrate, a family of substrate peptides with sequence similarities may be produced, e.g. a sequence identity of at least 60%, thereby producing a family of cyclic peptides comprising similar structures and motifs.

Alternatively, the similar structures and motifs may be produced by random integration of specific peptide sequences within a substrate peptide, e.g. at different positions, again producing a family of peptide sequences.

Different positions are e.g. resulting from Ser/Thr and/or Cys residues in any of the distal regions or the centric region of the substrate peptide. For example, it is preferred that at least one of the Ser/Thr and/or Cys residues is positioned within a distal region and the counterpart for forming the thioether bridge is positioned in the centric region or at the other distal region. The distal region may include the C-terminal or N-terminal amino acids up to ⅓ of the total sequence. The centric region typically includes the inner ⅓ of the total amino acid sequence of the substrate peptide.

The variety of cyclic structures within a library may include monocyclic, dicyclic, tricyclic and tetracyclic structures or even more complex structures, i.e. with one, two, three or four loops, up to ten loops per substrate peptide. If a dimeric, oligomeric or polymeric substrate peptide is used, the number of loops may increase accordingly. In particular, loops may be overlapping or a loop within a loop structure may be obtained.

The library of the invention may be suitable used for selecting active substances for pharmaceutical use (including therapeutic or diagnostic use), analytical, industrial, agricultural, pesticidal use (including agents for plant protection or pesticides), food or feed use (including food or feed additives or food preservatives).

Once a library member is selected which has a desired structure and function, the cyclic peptide may be produced in vivo using a biological expression system and the PTME enzyme, e.g. a host cell expressing the substrate peptide and optionally co-expressing the PTME, and/or by enzymatic transformation in vitro, e.g. incubating the substrate peptide and the PTME so that the enzymatic transformation may occur, or by organic or chemical synthesis methods.

### FIGURES

FIG. 1: Amino acid sequences

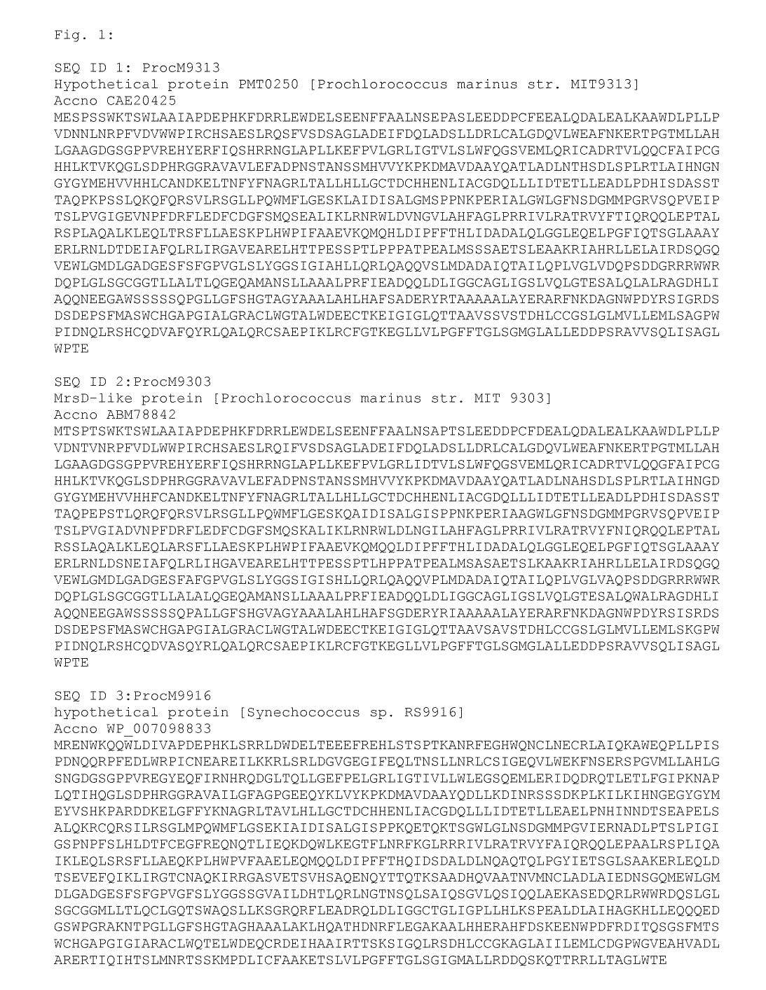

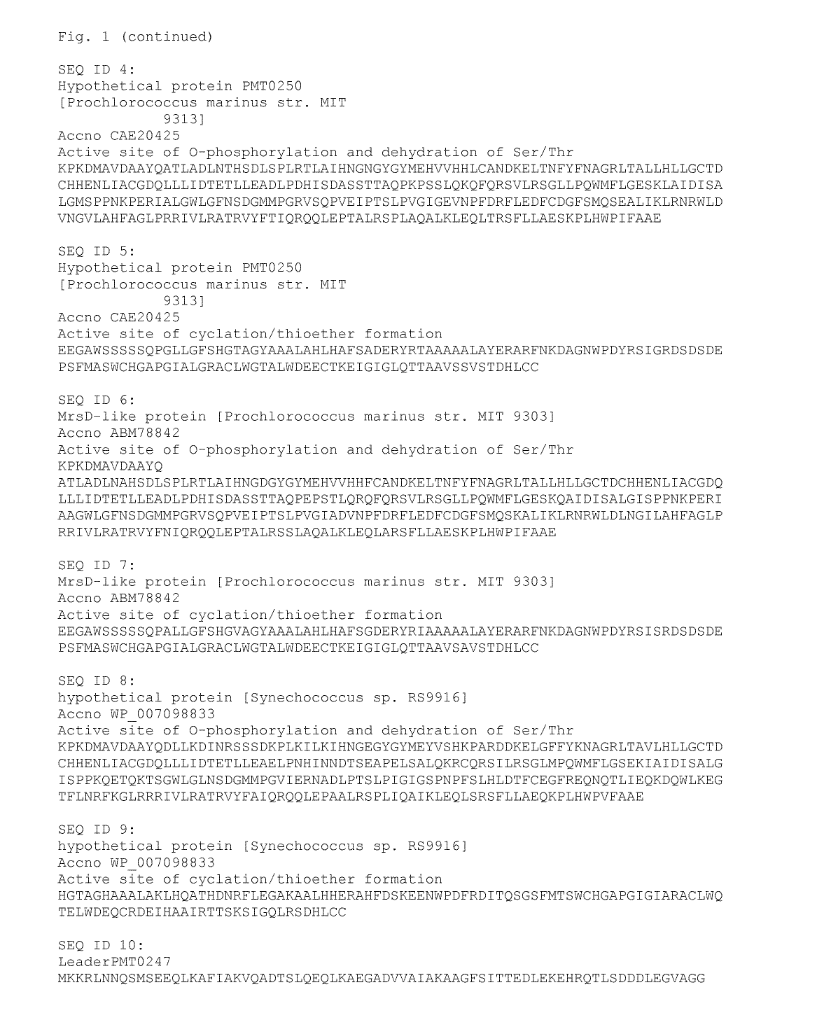

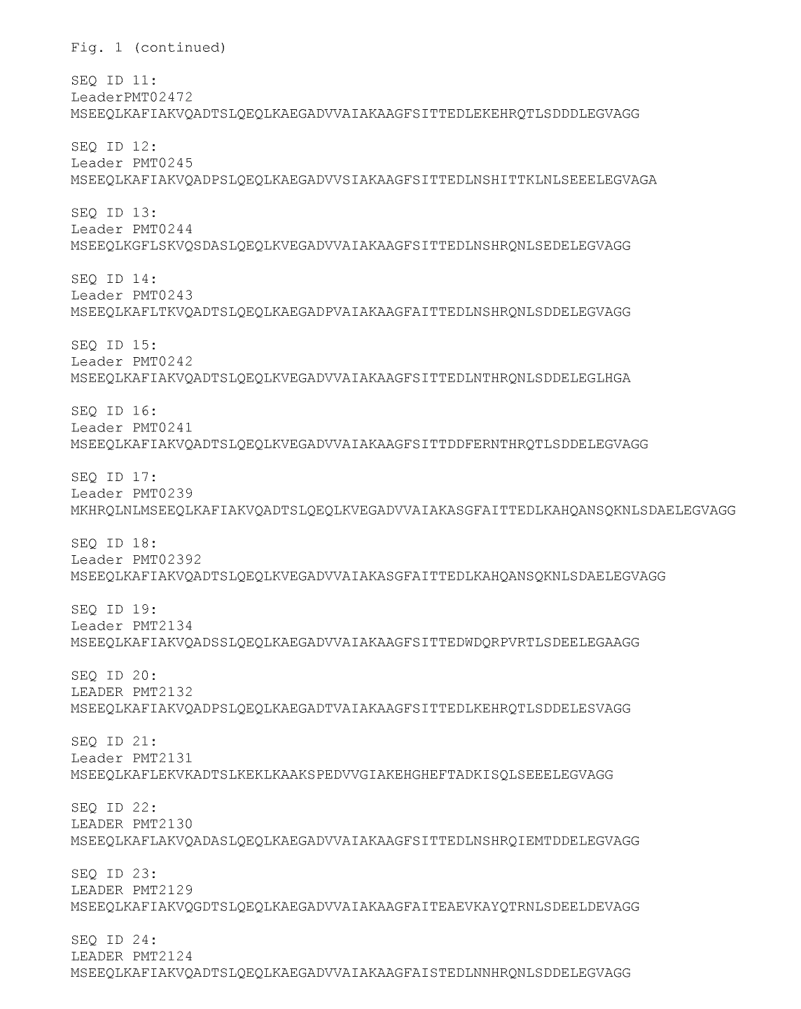

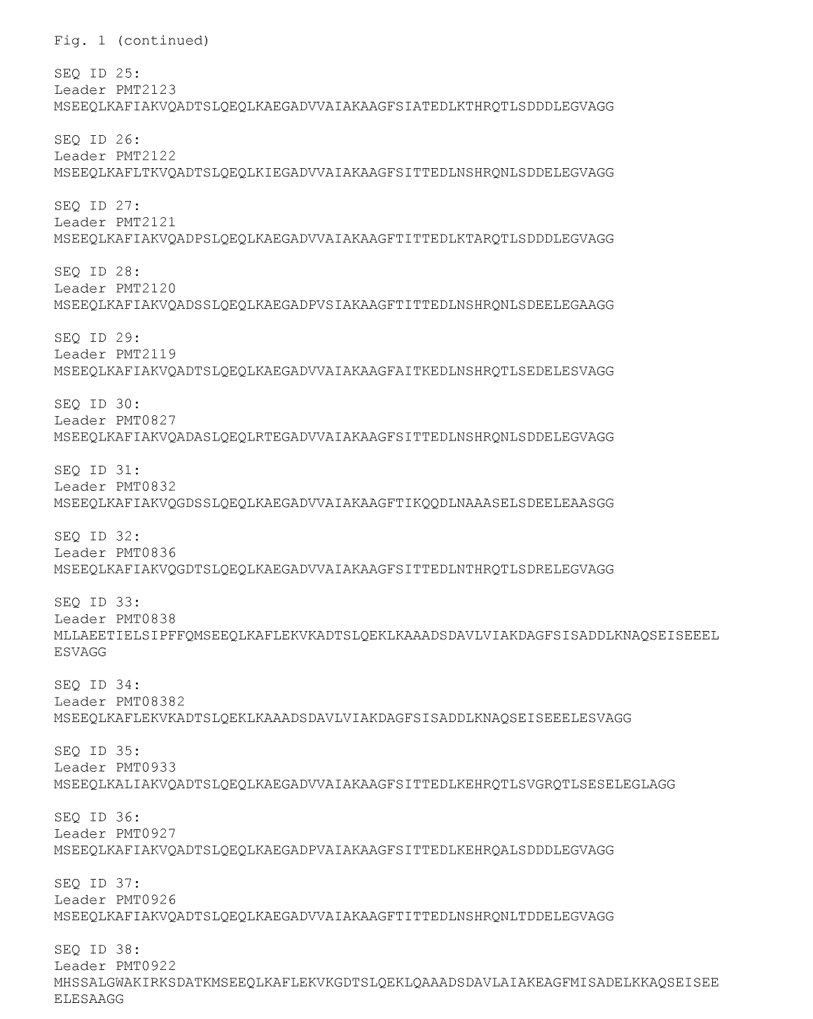

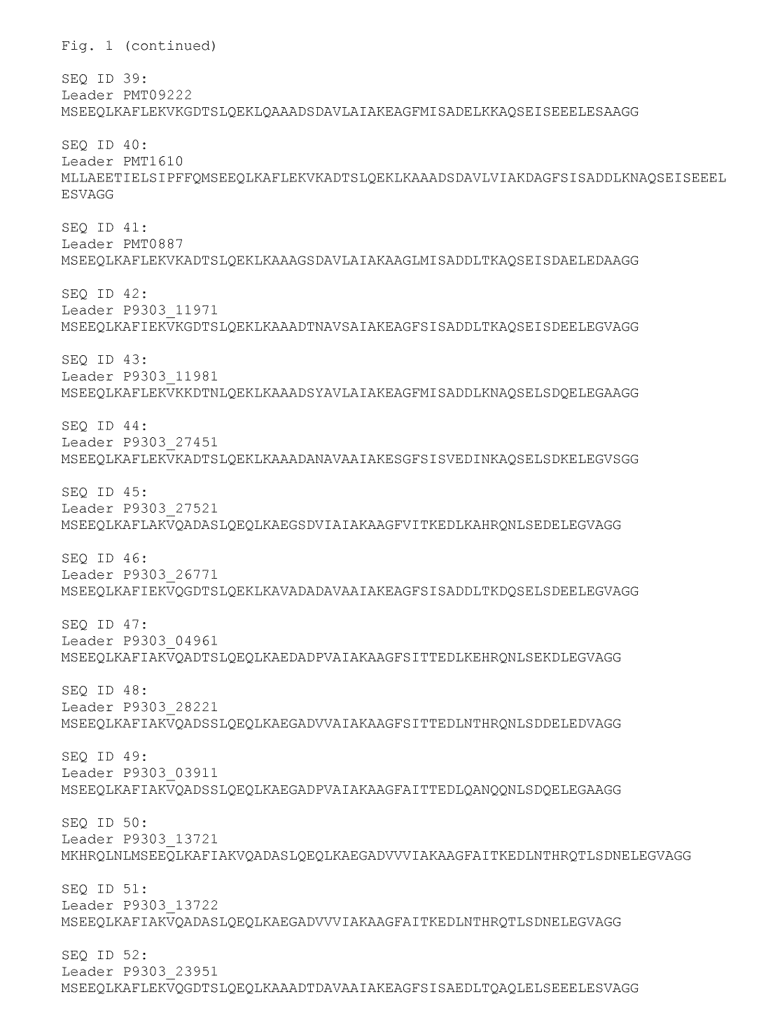

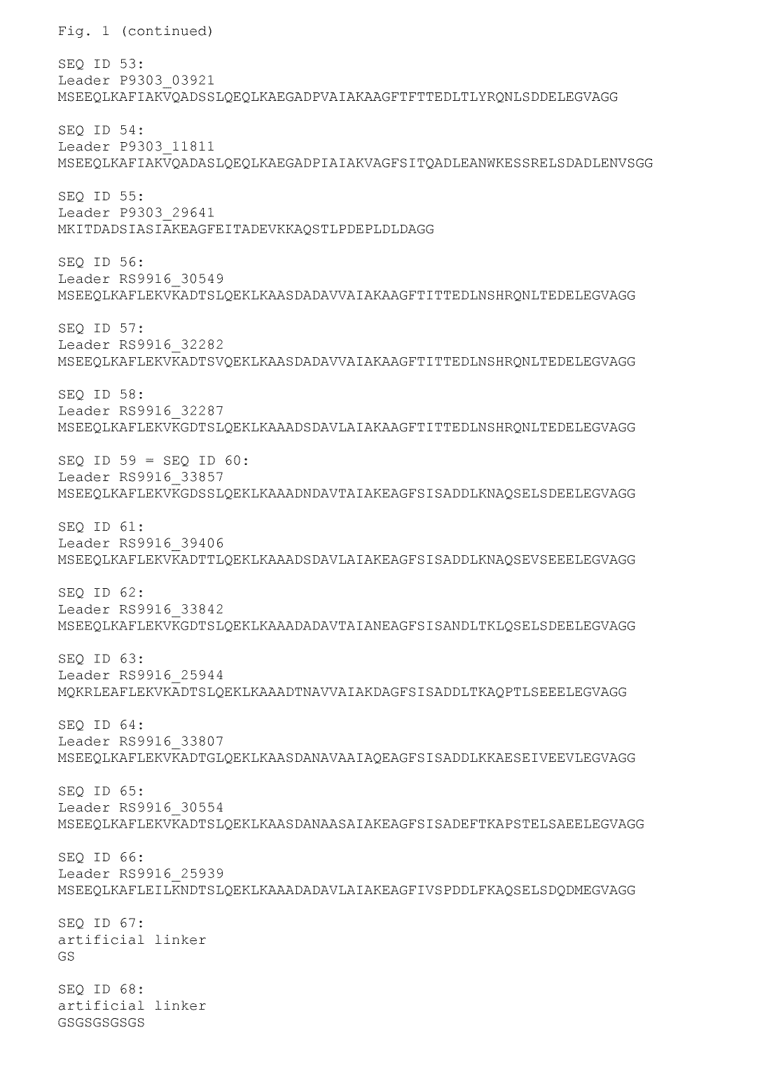

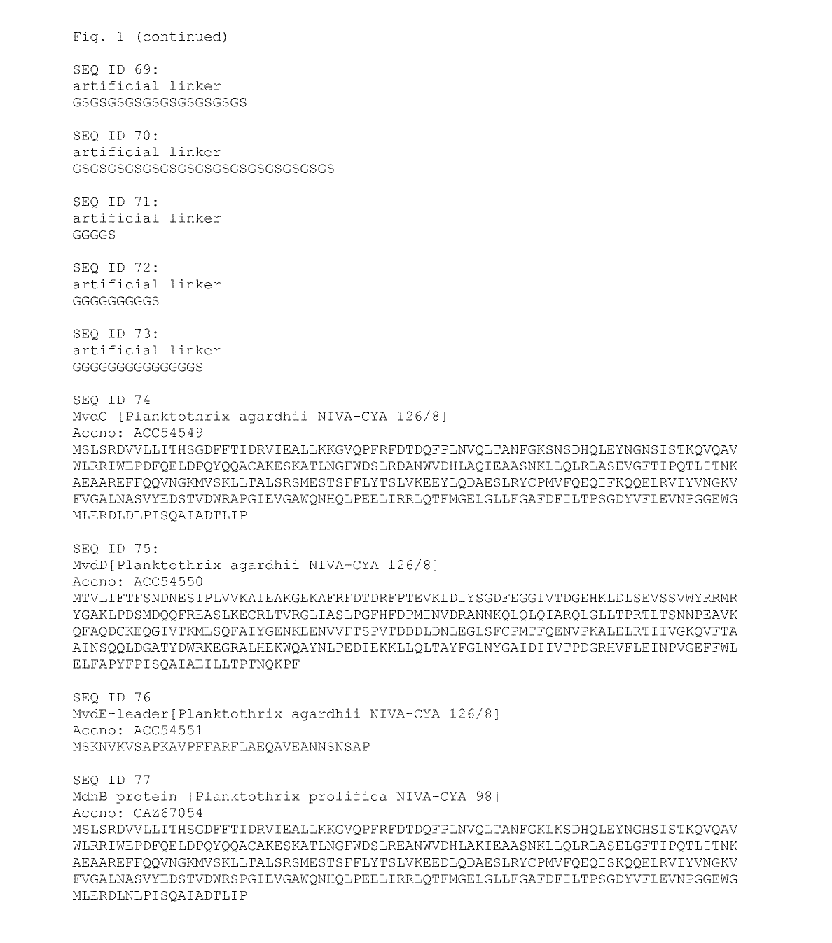

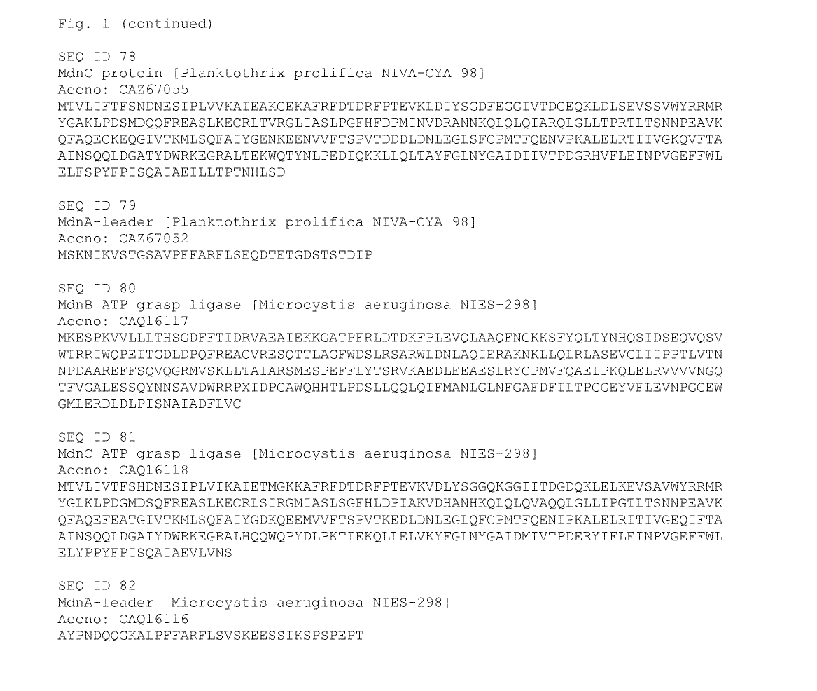

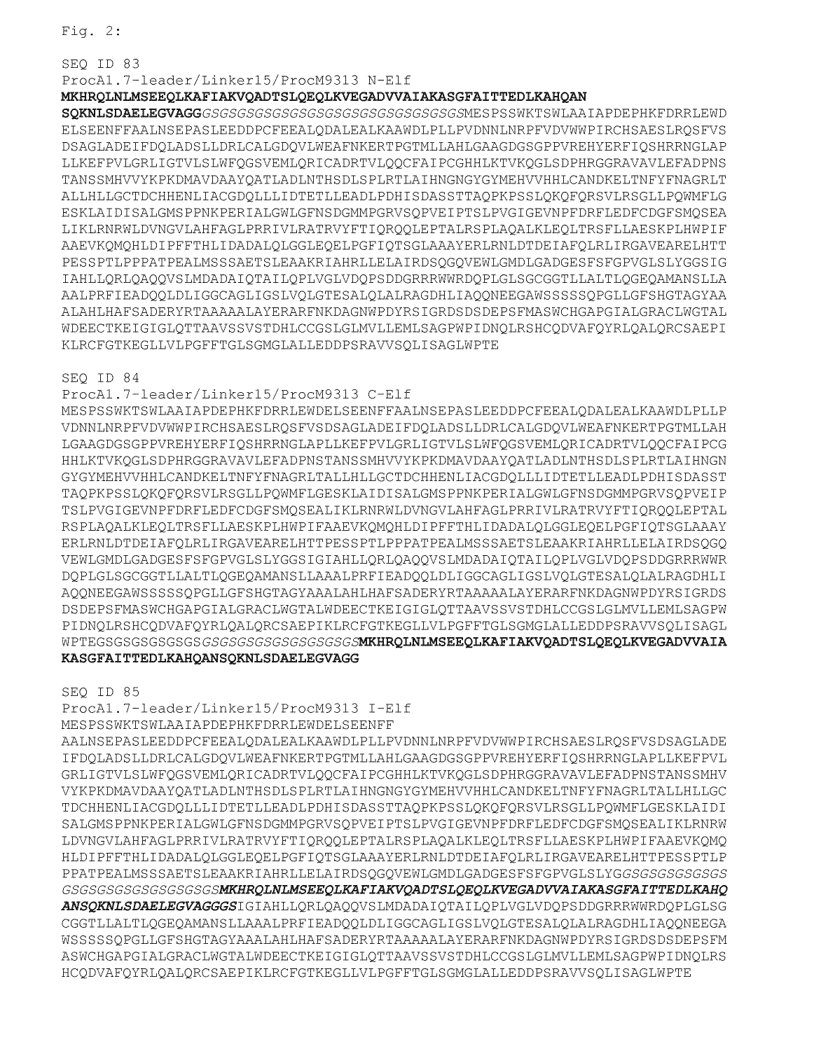

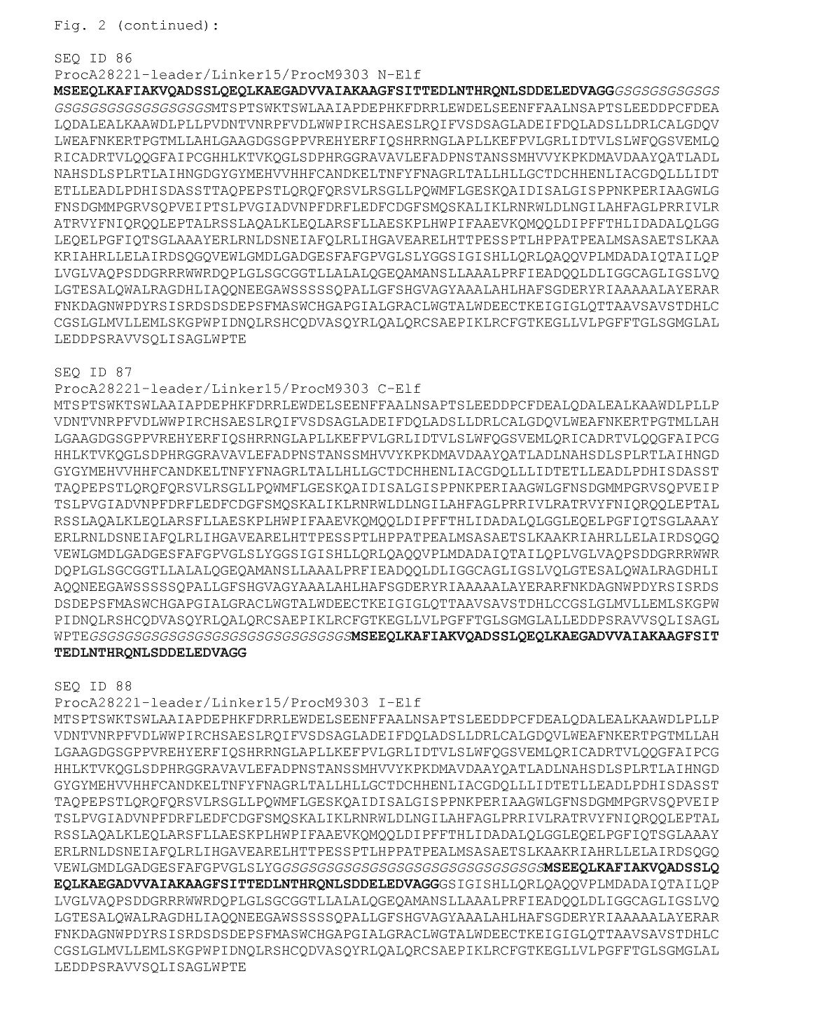

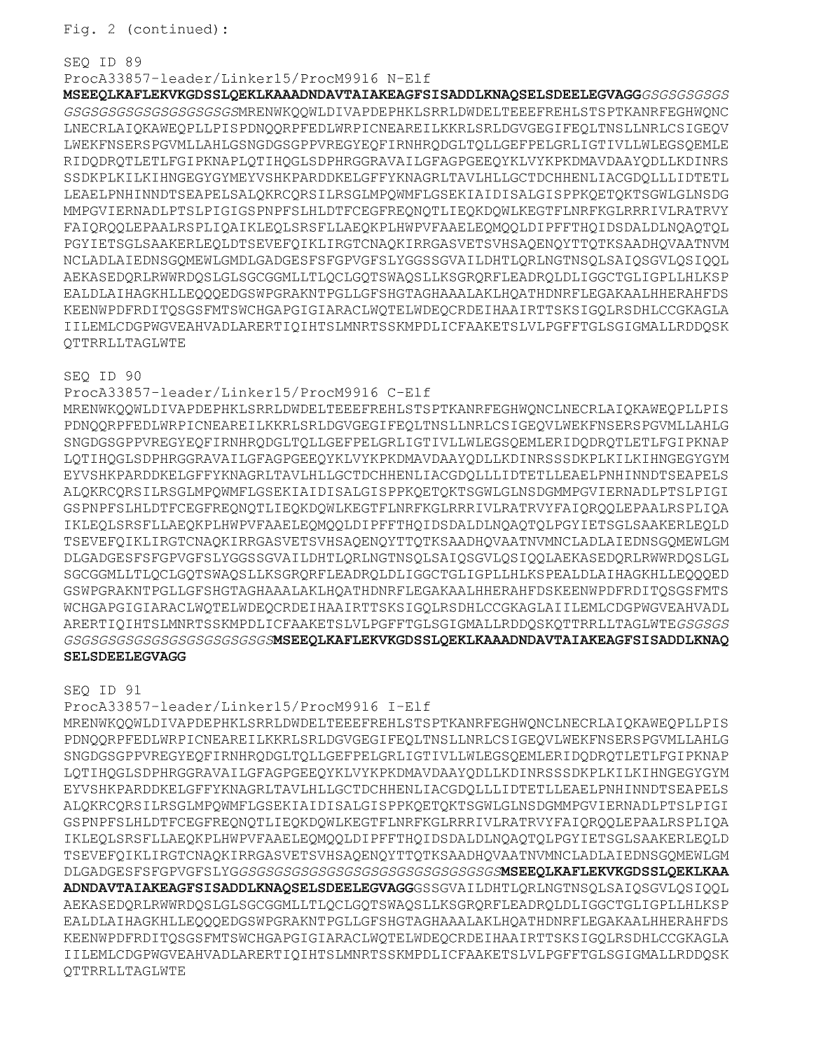

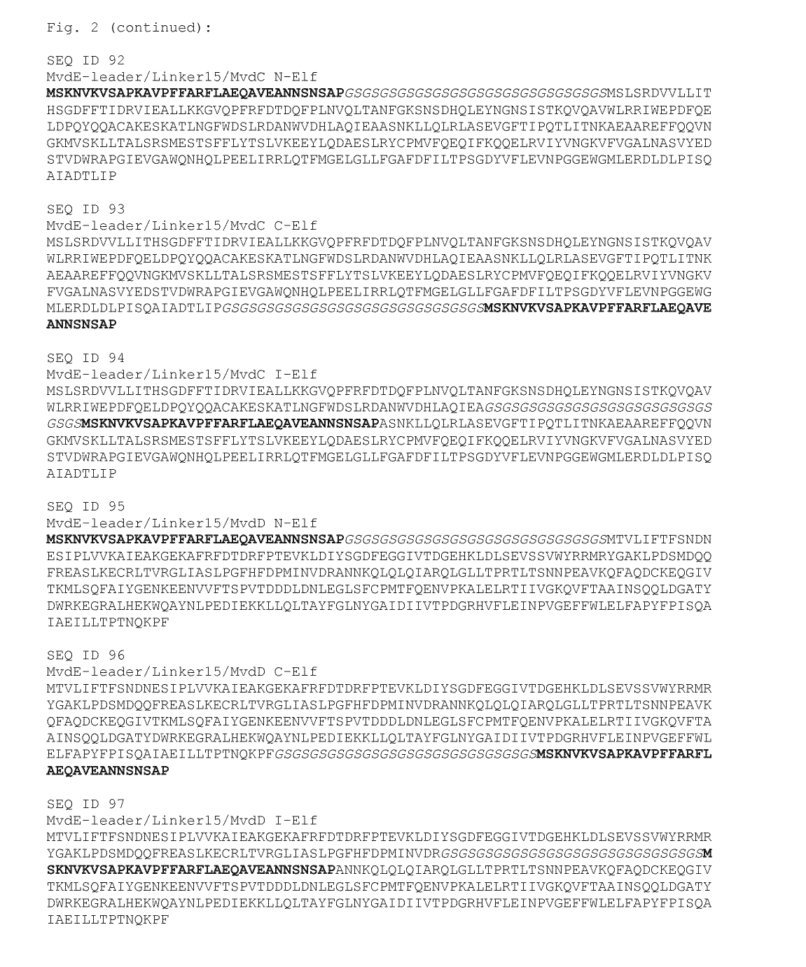

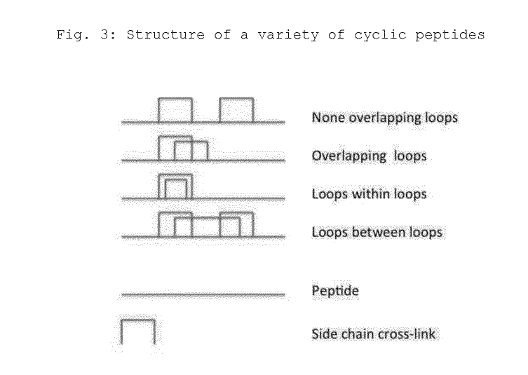

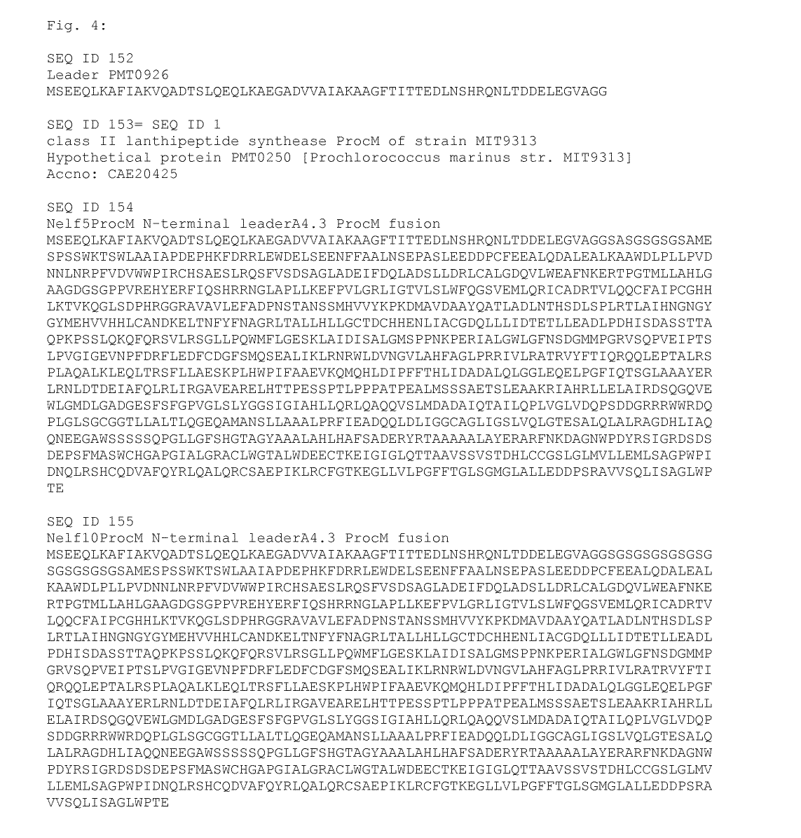

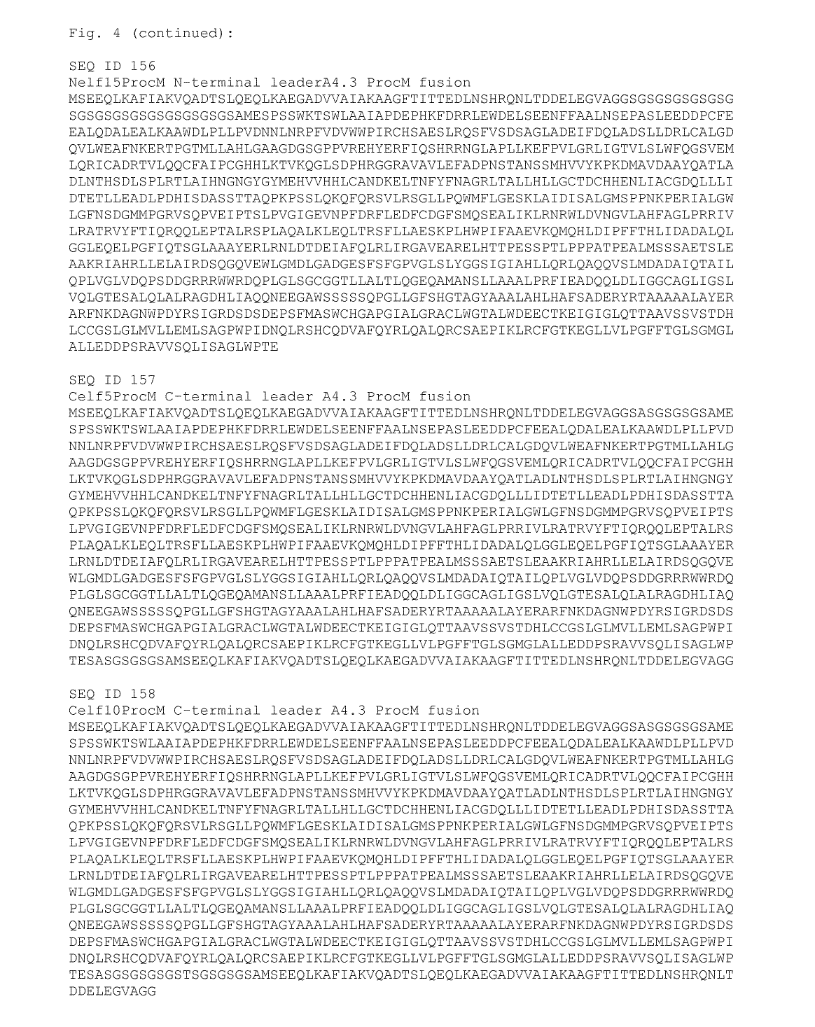

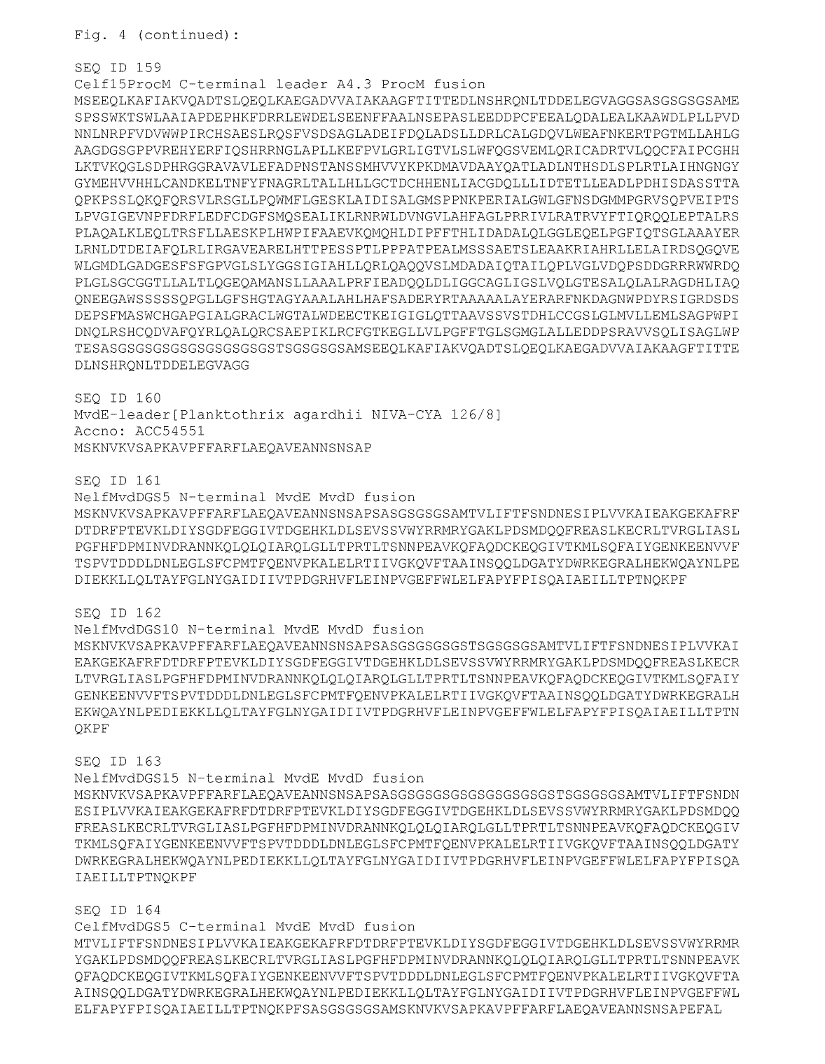

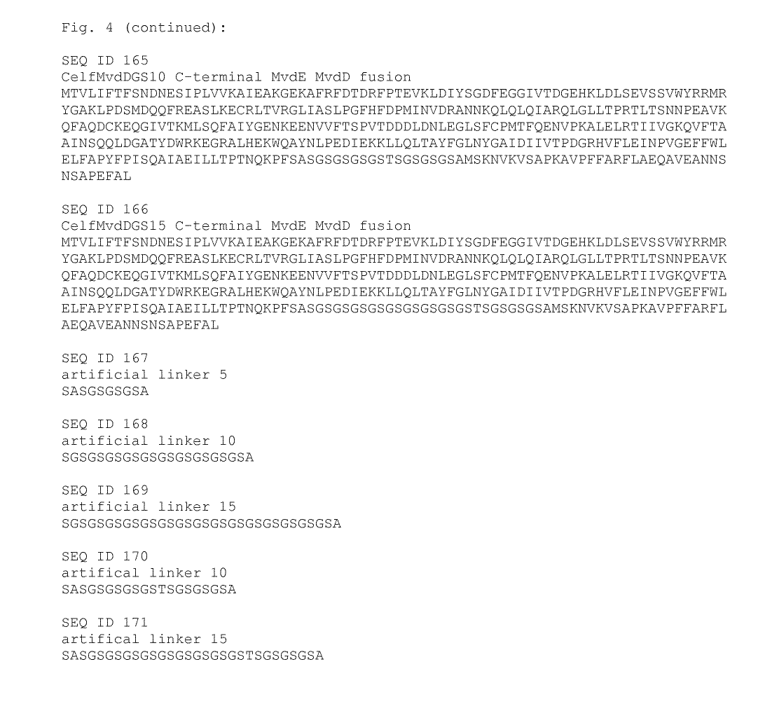

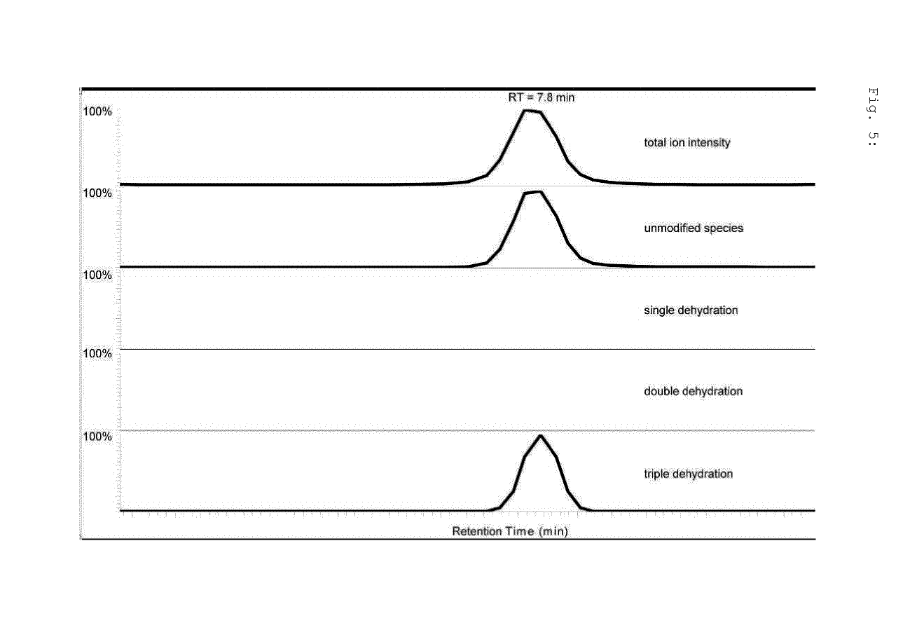

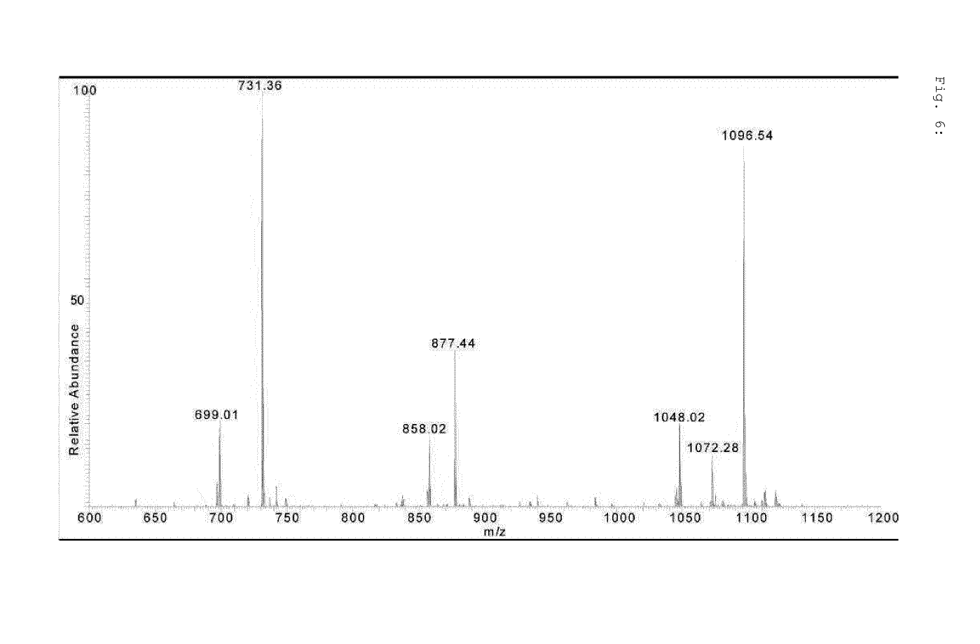

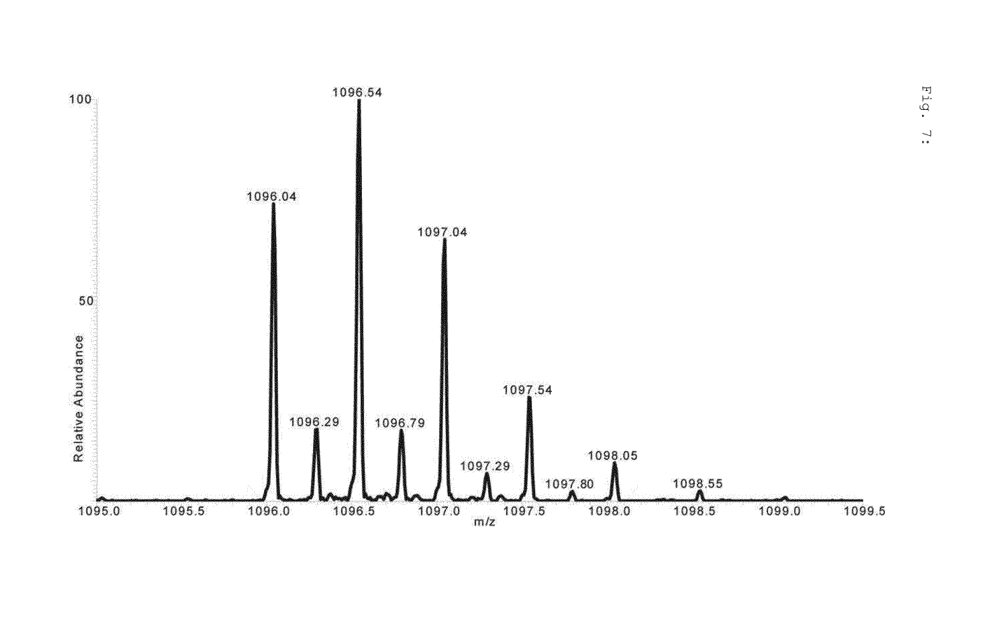

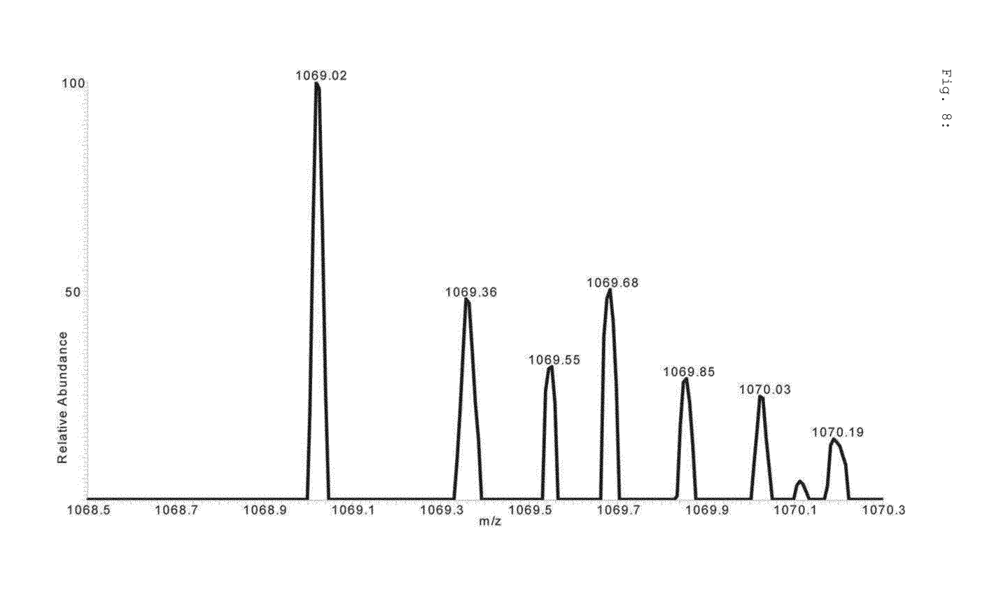

SEQ ID 1: ProcM9313 (Prochlorococcus marinus)

SEQ ID 2: ProcM9303 (Prochlorococcus marinus)

SEQ ID 3: ProcM9916 (Synechococcus sp.)

SEQ ID 4: active site dehyration ProcM9313

SEQ ID 5: active site cyclation ProcM9313

SEQ ID 6: active site dehyration ProcM9303

SEQ ID 7: active site cyclation ProcM9303

SEQ ID 8: active site dehyration ProcM9916

SEQ ID 9: active site cyclation ProcM9916

SEQ ID 10-41: cognate leader sequences of ProcM9313

SEQ ID 42-55: cognate leader sequences of ProcM9303

SEQ ID 48: leaderPMT0239, which is herein also referred to as ProcA1.7-leader

SEQ ID 56-66: cognate leader sequences of ProcM9916

SEQ ID 67-73: linker sequences: synthetic/artificial sequence

SEQ ID 74: MvdC (Planktothrix agardhii NIVA-CYA126/8)

SEQ ID 75: MvdD (Planktothrix agardhii NIVA-CYA126/8)

SEQ ID 76: LeaderMvdE(Planktothrix agardhii NIVA-CYA126/8)

SEQ ID 77: MdnB (Planktothrix prolifica NIVA-CYA 98)

SEQ ID 78: MdnC (Planktothrix prolifica NIVA-CYA 98)

SEQ ID 79: LeaderMdnA (Planktothrix prolifica NIVA-CYA 98)

SEQ ID 80: MdnB (Microcystis aeruginosa NIES-298)

SEQ ID 81: MdnC (Microcystis aeruginosa NIES-298)

SEQ ID 82: LeaderMdnA (Microcystis aeruginosa NIES-298)

FIG. 2: Amino acid sequences of exemplary ELFs:

bold: PTME-leader sequence;

Italic: linker sequence

other: PTME sequence

SEQ ID 83: ProcA1.7-leader/Linker15/ProcM9313 N-Elf

SEQ ID 84: ProcA1.7-leader/Linker15/ProcM9313 C-Elf

SEQ ID 85: ProcA1.7-leader/Linker15/ProcM9313 I-Elf

SEQ ID 86: ProcA28221-leader/Linker15/ProcM9303 N-Elf

SEQ ID 87: ProcA28221-leader/Linker15/ProcM9303 C-Elf

SEQ ID 88: ProcA28221-leader/Linker15/ProcM9303 I-Elf

SEQ ID 89: ProcA33857-leader/Linker15/ProcM9916 N-Elf

SEQ ID 90: ProcA33857-leader/Linker15/ProcM9916 C-Elf

SEQ ID 91: ProcA33857-leader/Linker15/ProcM9916 I-Elf

SEQ ID 92: MvdE-leader/Linker15/MvdC N-Elf

SEQ ID 93: MvdE-leader/Linker15/MvdC C-Elf

SEQ ID 94: MvdE-leader/Linker15/MvdC I-Elf

SEQ ID 95: MvdE-leader/Linker15/MvdD N-Elf

SEQ ID 96: MvdE-leader/Linker15/MvdD C-Elf

SEQ ID 97: MvdE-leader/Linker15/MvdD I-Elf

FIG. 3: Structure of a variety of cyclic peptides: polycyclic structures

First line: two serial loops, non-overlapping;

Second line: overlapping loops;

Third line: loop within a loop;

Fourth line: overlapping loop: two serial loops, one loop between loops;

Fifth line: symbol of linear peptide in this chart;

Sixth line: symbol of a loop (side chain cross-link).

FIG. 4: Sequences used in the Examples

SEQ ID 152 ProcA 4.3 leader sequence

SEQ ID 153 Class II lanthipeptide synthetase of ProcM

SEQ ID 154 Nelf5ProcM for N-terminal leaderA4.3 ProcM fusions

SEQ ID 155 Nelf10ProcM for N-terminal leaderA4.3 ProcM fusions

SEQ ID 156 Nelf15ProcM for N-terminal leaderA4.3 ProcM fusions

SEQ ID 157 Celf5ProcM for C-terminal leaderA4.3 ProcM fusions

SEQ ID 158 Celf10ProcM for C-terminal leaderA4.3 ProcM fusions

SEQ ID 159 Celf15ProcM for C-terminal leaderA4.3 ProcM fusions

SEQ ID 160 MvdE leader sequence

SEQ ID 161 NelfMvdDGS5 for N-terminal MvdE MvdD fusions

SEQ ID 162 NelfMvdDGS10 for N-terminal MvdE MvdD fusions

SEQ ID 163 NelfMvdDGS15 for N-terminal MvdE MvdD fusions

SEQ ID 164 CelfMvdDGS5 for C-terminal MvdE MvdD fusions

SEQ ID 165 CelfMvdDGS10 for C-terminal MvdE MvdD fusions

SEQ ID 166 CelfMvdDGS15 for C-terminal MvdE MvdD fusions

SEQ ID 167-171: artificial linker sequences

FIG. 5: Ion chromatograms of reduced, desalted sample. Top trace1: Total ion chromatogram (TIC), trace2: extracted ion chromatogram (EIC) of unmodified peptide P2, trace3: EIC of P2 with 1× dehydration, trace4: EIC of P2 with 2× dehydrations, trace5: EIC of P2 with 3× dehydrations.

FIG. 6: Mass spectrum sample at RT 7.5 min showing the different peptide species with unmodified peptide P2 at 1096.041 m/z, annotated with its second isotope signal 1096.542 m/z.

FIG. 7: Zoomed mass spectrum sample at RT 7.5 min with unmodified peptide P2 1096.041 m/z=[M+2H+]2+.

FIG. 8: Zoomed mass spectrum sample at RT 7.5 min with peptide P2 after 3× dehydrations 1069.022 m/z=[M+2H+]2+(nominal isotope m/z-values: 1069.025 m/z, 1069.526 m/z, 1070.028 m/z).

### DETAILED DESCRIPTION OF THE INVENTION

The term “biosynthesis” of a cyclic peptide, or “biotransformation” of a substrate peptide into a cyclic peptide is herein understood as the enzymatic reaction or transformation to catalyze the conversion of a substrate peptide into the peptide having the desired cyclic peptide structure. The enzymatic reaction may be carried out by an organism, in cell culture, e.g. by a host cell culture or by a lysate of said host cell (e.g. a cell-free lysate), wherein the host expresses either of the substrate or the enzyme, or co-expresses both, so that the enzymatic reaction occurs in vivo, namely in the host cell culture. Alternatively, the enzymatic reaction may be carried out in vitro, by incubating both, the substrate and the enzyme, in a suitable medium, without employing a host cell.

The term “cyclic peptide” as used herein shall mean a peptide which is characterized by a secondary structure formed through intramolecular covalent bonds, which employ covalent bonding between side chains of amino acids within a peptide sequence, or at least between a side chain of an amino acid residue and the peptide chain, e.g. an a amino group may be linked with a ω carboxyl group, e.g. without incorporating extramolecular (exogenous) structures. Such cyclic peptide comprises one or more intramolecular bonds or bridges cross-linking the amino acid residues in an amino acid sequence, thereby forming a cycle or loop. Those amino acids which are connected by their amino acid side chains are understood as bridge-piers. By such cross-linking, the peptide gets a three-dimensional, constrained structure, thereby providing new contact surfaces for interactions with reaction partners. Intramolecular bridges may be bridges connecting two carbon atoms (C—C), but also heteroatom bridges, e.g. by a bond between C and a heteroatom (other than C), or between two heteroatoms of the same kind (other than C—C) or different kinds, thereby forming heterocycles.

The length of the loops increases with the number of spanning amino acids between the bridge-piers of the bridge. Depending on the number of potential bridge piers in a peptide sequence, the number of loops may increase, e.g. to provide a polycyclic structure.

The term “cyclic peptide” shall specifically refer to those structures that have been obtained through post-translational enzymatic processing, which would preferably exclude chemical processing, such as disulfide bridge formation, e.g. through reduction reaction, cycloaddition or Staudinger reactions. Specifically the cycle is a heterocycle including at least two atoms herein called “heteroatoms”, which are either heteroatoms, such as N, O or S, or atoms forming a covalent bond between two different atoms selected from the group consisting of C, N, O and S. By cross-linking two heteroatmoms, a “heteroatom bridge” or “heterocycle” is formed, e.g. heterocycles produced by post-translational modification or metabolic processing. This specifically includes C—N, C—O, C—S, N—N, N—O, N—S, O—O, O—S and S—S bonding, in an appropriate chemical sense including double bonds. Specific examples of heterocycles are formed by a thioether bridge formation, linking C—S, thereby forming a C—S—C bridge.

The isolated and purified cyclic peptide can be identified by conventional methods such as Liquid chromatography-mass spectrometry (LC-MS or HPLC-MS), fourier transformed mass spectrometry (FT-MS), HPLC, activity assay, Western blot, or ELISA.

The term “polycycle” or “polycyclic structure” as used herein shall refer to at least a bicyclic structure, which comprises two loops, preferably a structure having at least three, more preferred at least four, even more preferred at least five loops within the amino acid sequence of a peptide. Depending on the length of the peptide as used according to the invention a more complex secondary peptide structure can be achieved. Such loops may be positioned sequentially and/or overlapping, e.g. wherein the amino acid sequence within a first loop comprises one bridge-pier of a second loop, while the other bridge-pier of the second loop is positioned outside the first loop. If the second loop is positioned within the first loop, the loops structure is called “loop in a loop”.

The term “displaying genetic package” as used herein shall mean a unit comprising an inner and an outer part, wherein the inner part comprises a nucleic acid sequence and the outer part the translated amino acid sequence.

The genetic package is typically a compartment, e.g. part of a biological translational system like a ribosome, polysome, emulsion compartment or a vesicle; an artificial construct like an RNA/DNA-fusion protein (covalent display), the display on a plasmid, or CIS display; or a prokaryotic, eukaryotic or viral genetic package, including cells, spores, yeasts, bacteria, viruses, or bacteriophages. A preferred genetic package is a phage. Phage display is usually based on DNA libraries fused to the N-terminal end of filamentous bacteriophage coat proteins and their expression in a bacterial host resulting in the display of foreign peptides on the surface of the phage particle with the DNA encoding the fusion protein packaged in the phage particle. While N-terminal fusions are commonly used, C-terminal fusions may be done as well.

While cellular or viral genetic packages are commonly understood as “in vivo display” or “biological display” systems, the artificial display systems which do not rely on in vivo expression, like partial biological translation systems (e.g. ribosomes) or artificial constructs are understood as “in vitro display” systems. Any of the genetic packages of in vivo or in vitro display systems is also understood as a “replicable genetic package”. Contrary to in vitro display systems, the biological systems typically employ a viral or cellular expression system, e.g. expressing a library of nucleic acids in transformed, infected, transfected or transduced cells and display of the encoded peptides on the surface of the genetic package.

The nucleic acid molecule of a genetic package usually is replicable either in vivo, e.g. as a vector, or in vitro, e.g. by PCR, transcription and translation. In vivo replication can be autonomous such as for a cell, with the assistance of host factors, such as for a virus, or with the assistance of both host and helper virus, such as for a phagemid. Replicable genetic packages displaying a variety of peptides are formed by introducing nucleic acid molecules encoding heterologous peptides to be displayed into the genomes of the replicable genetic packages to form fusion proteins with autologous proteins that are normally expressed at the outer surface of the replicable genetic packages. Expression of the fusion proteins, transport to the outer surface and assembly results in display of the peptides from the outer surface of the replicable genetic packages.

Genetic packages are typically immobilising the translated product, e.g. binding to a specific compartment comprising the nucleic acid. Binding to the genetic package may be through covalent binding, e.g. by fusion to a linking element, such as a coat protein or a peptide linker, or by linking to a side chain of an amino acid, or by disulfide bonds between two cysteins (Cys-display).

The genetic package as used herein can be a screenable unit comprising a peptide to be screened linked to a nucleic acid molecule encoding the peptide. The peptides can be immobilised and displayed by the genetic package carrying the peptide, i.e. they are attached to a group or molecule located at an outer surface of the genetic package. Such genetic packages presenting the peptides on its surface are commonly understood as “surface display system”.

The display system as used according to the invention usually refers to a collection of peptides that are accessible for selection based upon a desired characteristic, such as a physical, chemical or functional characteristic, whereupon a nucleic acid encoding the selected peptide can be readily isolated or recovered. The display system preferably provides for a suitable repertoire of peptides in a biological system, sometimes called a biological display system, which specifically refers to replicable genetic packages.

The term “functionally active variant” of a parent molecule as used herein means a sequence resulting from modification of this sequence by insertion, deletion or substitution of one or more amino acids or nucleotides within the sequence or at either or both of the distal ends of the sequence, and which modification does not affect (in particular impair) the activity of this sequence.

In the case of a target binding peptide having specificity to a selected ligand, the functionally active peptide variant as used according to the invention would still have the predetermined binding specificity, though this could be changed, e.g. to change the fine specificity to a specific epitope, the affinity, the avidity, the Kon or Koff rate, etc.

In the case of an enzyme with catalytic activity, the functionally active variant as used according to the invention would still have the predetermined enzymatic activity, though this could be changed to change or improve its function. In particular, one or more point mutations may be introduced besides the catalytic site, which are herein also understood as active sites. Those modifications aside from the active site are typically less critical than modifications within the active site, which could reduce the catalytic activity. Thus, a functionally active variant of an enzyme could comprise the catalytic site of a parent enzyme, e.g. with one or more point mutations and a sequence identity of at least 60%, preferably at least 70%, or at least 80% or at least 90% as compared to the catalytic site of the parent enzyme; and further regions which differ from the comparable regions of the parent molecule, such that the sequence identity of the full-length molecule is less than 60%, e.g. a total exchange of domains aside from the active site, or with a sequence identity of at least 60%, preferably at least 70%, or at least 80% or at least 90% as compared to the sequence of the parent molecule. If the parent enzyme is a bifunctional enzyme comprising two catalytic sites, a functionally active variant may be engineered by modifying one or both of the catalytic sites. If both catalytic sites are modified, the functionally active variant still maintains the catalytic activity of at least one of the active sites.

In a preferred embodiment the functionally active variant a) is a functionally active fragment of the parent molecule, the functionally active fragment comprising at least 50% of the sequence of the parent molecule, preferably at least 60% or 70%, more preferably at least 80%, still more preferably at least 90%, even more preferably at least 95% and most preferably at least 97%, 98% or 99%; b) is derived from the parent molecule by at least one amino acid substitution, addition and/or deletion, wherein the functionally active variant has a sequence identity to the peptide of at least 50%, preferably at least 60%, more preferably at least 70%, more preferably at least 80%, still more preferably at least 90%, even more preferably at least 95% and most preferably at least 97%, 98% or 99%. The functionally active variant may be a size variant, such as a fragment or elongated sequence, e.g. extended by either N-terminal or C-terminal or internal fusions, or a combination of such fusions.

The variant of the polypeptide or the nucleotide sequence is typically a non-naturally occurring variant of an original (naturally-occurring) parent molecule, and functionally active in the context of the present invention, e.g. if a substrate peptide is still eligible to loop formation, or if a PTME-leader peptide is still being recognized by a PTME-enzyme similar to the parent PTME-leader, i.e. the original leader without modification, e.g. a naturally-occurring PTME-leader, or if the activity of a PTME preparation still has enzymatic activity, such as to the extent of at least 10%, preferably at least 25%, more preferably at least 50%, even more preferably at least 70%, still more preferably at least 80%, especially at least 90%, particularly at least 95%, most preferably at least 99% of the activity of the parent PTME, i.e. the original enzyme without modification, e.g. a naturally-occurring PTME.

An enzyme linked to its recognition sequence, e.g. an enzyme-leader fusion protein is herein understood as a functionally active variant of the enzyme without such fusion to its leader sequence. An exemplary enzyme-leader fusion is herein understood as a PTME enzyme linked to its cognate PTME-leader peptide, thereby obtaining an enzyme-leader fusion molecule, abbreviated as ELF.

Functionally active variants may be obtained by sequence alterations in a parent sequence, e.g. the amino acid or the nucleotide sequence, wherein the altered sequence retains a function of the unaltered sequence, when used in combination of the invention. Such sequence alterations can include, but are not limited to, (conservative) substitutions, additions, deletions, mutations and insertions. Typical sequence alterations include point mutations.

Specific embodiments of a point mutation lead to any of an amino acid substitution, deletion and/or insertion of one or more amino acids. Such point mutations may increase the frequency of potential bridge piers that could form a heteroatom bridge and a loop to obtain a cyclic peptide. Alternatively, point mutations may lead to elongating or shortening the loop length by inserting or deleting amino acids between bridge piers, or by deleting possible bridge piers within a substrate peptide.

Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

In another embodiment of the invention a parent sequence as defined above may be modified by a variety of chemical techniques to produce derivatives having essentially the same activity (as defined above for fragments and variants) as the original molecule, and optionally having other desirable properties.

“Percent (%) amino acid sequence identity” with respect to sequences identified herein is defined as the percentage of amino acid residues in a candidate sequence that are identical with the amino acid residues in a specific comparable polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

The term “repertoire” as used herein refers to a collection of nucleic acid or amino acid sequences that are characterized by sequence diversity. The individual members of a repertoire may have common features, such as a common core structure within a scaffold, and/or a common function, e.g. a specific binding or biological activity. Within a repertoire there are usually “variants” of a nucleic acid or amino acid sequence, such as a variety of peptide sequences, which are derived from a parent sequence through mutagenesis methods, e.g. through randomisation techniques. The term “library” as used herein refers to a mixture of heterogeneous peptide or nucleic acid sequences. The library is composed of members, each of which has a single peptide or nucleic acid sequence. To this extent, “library” is synonymous with “repertoire.” Sequence differences and differences in the cyclic structure between library members are responsible for the diversity present in the library.

The term “randomisation” or “randomised sequence” shall refer to specific nucleotide or amino acid sequence modifications in a predetermined region, e.g. forming new bridge piers of heterocycles upon enzymatic reaction or metabolic processing, or between such bridge piers changing the three-dimensional structure of the heterocycle. Modification typically results in random insertion, exchange or deletion of amino acids, e.g. point mutations. For substituting or inserting amino acids a selection of amino acids or the whole range of natural or synthetic amino acids may be used randomly or semi-randomly by methods known in the art and as disclosed in the present patent application. Randomisation will result in a repertoire of nucleic acids encoding a variety of peptide sequences. The use of natural amino acids is preferred for randomisation purposes. In specific embodiments natural amino acids may be used, e.g. to produce non-natural amino acids like oxazole/oxazoline or thiazol/thiazoline structures.

Partial randomisation refers to randomisation of only a part of a molecule, e.g. aside from critical regions such as existing bridge piers of a heterocycle within a substrate peptide, or aside from the catalytic site of an enzyme, or aside from the recognition sequence of a leader sequence. Full randomisation refers to mutagenesis of any site in a molecule, e.g. a full randomisation of a peptide leads to variants, wherein any position could be modified, e.g. to place any of the natural amino acid residues in any position, with or without size variation. Randomisation techniques may consider strategies to increase the frequency of desired amino acids in a sequence to be randomised. For example, a proportion of at least one base at a specified position in a codon may be varied to bias the codon towards coding for a desired amino acid sequence. Such bias may lead to the increase or decrease of the number of bridge piers of a heterocycle in a substrate peptide, e.g. Ser or Thr or Cys residues to change the number of thioether bridges within the cyclic peptide obtained upon enzymatically processing of the randomised substrate peptide. By randomisation of a substrate peptide, “random” cyclic peptides may be obtained, i.e. a repertoire of cyclic peptides that are randomised to include the variety of sequence variants and variants with different cyclic structure.

The term “origin” with respect to PTME or PTME-leader or ribosomal (substrate) peptides is herein understood as the source of such substances or the respective nucleic acid encoding such substances. The origin may be a wild-type organism, producing the substance, and the wild-type nucleic acid sequence, respectively. In addition the originating substance may be a mutant of such wild-type, thereby producing a mutant or descent of the same origin. The PTME and the PTME-leader may be of the same or different origin, thus, be derived from the same organism and optionally including modifications to obtain a mutant PTME and/or a mutant PTME-leader. If the PTME and the PTME-leader is of a different origin, the substances originate from different species or genus, or at least one of the substances is artificial, e.g. with less than 50% sequence identity to a naturally-occurring substance, or rationally designed, thus, not originating from a wild-type organism.

For example, the cognate pair is of the same or different origin, preferably wherein at least one of the PTME-leader peptide or the PTME is naturally-occurring or a functionally active derivative thereof comprising one or more point mutations, preferably of a prokaryotic or eukaryotic origin, e.g. selected from the group consisting of Bacillus sp, Nonomuraea sp, Lactococcus sp, Escherichia sp, Streptomyces sp, Klebsiella sp, Clostridium sp, Actinoplanes sp, Staphylococcus sp, Actinomadura sp, Streptoverticillium sp, Leifsonia sp, Mycobacterium sp, Nitrospira sp, Solibacter sp, Haloterrigena sp, Streptococcus sp, Micrococcus sp, Micromonospora sp, Salinospora sp, Planobispora sp, Rhodococcus sp, Burkholderia sp, Asticcacaulis sp, Sorangium sp, Alteromonas sp, Enterococcus sp, Butyrivibrio sp, Lactobacillus sp, Carnobacterium sp, Leuconostoc sp, and Conus sp, Amanita sp, Galerina sp, Lepiota sp, Conocybe sp, Oldenlandia sp, Violaceae, Rubiaceae, Fabaceae, and Curcubitaceae.

Preferably, the cognate pair is of either prokaryotic or eukaryotic origin, wherein the pair is e.g. of the same genus or species.

The term “peptide” as used herein shall mean a peptide or polypeptide that contains 5 or more amino acids, typically at least 10, preferably at least 20, more preferred at least 30, more preferred at least 40, more preferred at least 50, more preferred at least 60, 70, 80, 90 or 100 amino acids. The term also refers to higher molecular weight polypeptides, such as proteins.

The term “post-translationally modifying enzyme” or “PTME” as used herein shall refer to enzymes involving structural changes of a translated peptide, e.g. specifically modifying natural ribosomal peptides in the biosynthesis of biologically active peptides as part of the processing machinery. This class includes multiple types of enzymes, including bifunctional dehydratase-cyclase, dehydratase, cyclase, carboxylate-amine ligase, decarboxylase, epimerase, hydroxylase, peptidase, dehydratase, transferase, esterase, oxygenase, isomerase and transglutaminase (e.g. microbial transglutaminase, mTG).

Specifically, a bifunctional dehydratase-cyclase would generate a thioether bridge between a Ser and a Cys or a Thr and Cys, thus form a S—C bond. Examples of such PTME are LanM type PTME, including any of the ProcM enzymes described herein, or LanM (AAU25567.3).

Specifically, a dehydratase would generate Dha (2,3-didehydroalanine) from Ser and Dhb ((Z)-2,3-didehydrobutyrine) from Thr. Examples of such PTME are NisB (CAA48381) and LanB (AFE10374).

Specifically, a cyclase catalyses the Michael-type addition of a cysteinyl thiol to a Dha or Dhb residue, thus forming an S—C bond. Examples of such PTMEs are NisC (CAA48383) and LanC (AGY89934).

Specifically, a carboxylate-amine ligase would generate a lactam bond between a Lys and an Asp or a Glu. An example of such a PTME is MvdC (ACC54549).

Specifically, a decarboxylase would remove the C-terminal carboxyl group of C-terminal cysteine through oxidative decarboxylation to a [Z]-enethiol structure, as part of the aminovinylcysteine formation. An example of such a PTME is LanD (ACD99095).

Specifically, an epimerase would generate D-amino acid residues from L-amino acid residues. An example of such a PTME is PoyD (AFS60640).

Specifically, a hydroxylase could generate erythro-3-hydroxy L-aspartic acid from aspartic acid. An example of such a PTME is CinX (CAD60522).

Specifically, a peptidase is a protease that would remove the N-terminal leader from a peptide. An example of such a PTME is McjB (AAD28495).

Specifically, a cyclodehydratase converts amino acids with a beta-necleophile (Cys, Ser or Thr) into thiazoline or (methyl)oxazoline rings. An example of such a PTME is McbB (CAT00698).

Specifically, a dehydrogenase oxidizes azoline to the aromatic azole heterocycle. An example of such a PTME is McbC (CAT00697).

Specifically, a transferase would generate an N-terminally acetyl capped peptide. An example of such a PTME is MvdB (ACC54548).

Specifically, a methyl transferase would generate N-methylated Asn from Asn. An example of an N-methyl transferase is PoyE (AFS60641).

Specifically, an esterase would hydrolyse the lactone scaffold of Microcin E492m. An example of such a PTME is MceD (AAL08397).

Specifically, a monooxygenase would be responsible for thiazoline methylation and phenylalanin β-hydroxylation during thiomuracin biosynthesis. An example of such PTME is TpdJ1 (ACS83778) and TpdJ2 (ACS83779).

Specifically, an isomerase (protein disulfide isomerase) would be involved in oxidative protein folding of conotoxins. An example of such a PTME is the protein disulfide isomerase MrPDI (ABF48564).

Specific examples are lanthionine bond forming enzymes, cytolysin forming enzymes, cyanobactin forming enzymes, thiopeptide forming enzymes, conopeptide forming enzymes, microviridin forming enzymes, cyclotide forming enzymes, bacteriocin forming enzymes, subtilosin forming enzymes, linearidin forming enzymes, proteusin forming enzymes, bottromycin forming enzymes, microcin forming enzymes, lasso peptide forming enzymes, amatoxin/phallotoxin forming enzymes, or sactipeptide forming enzymes.

Specifically, lanthionine bond forming enzymes catalyse the thioether bridge formation, thus, a C—S bond. Specific examples are Proc M (CAE20425) or LanM (AAU25567).

Specifically, sactipeptide forming enzyme catalyse the formation of a thioether bridge between cysteine sulphur and the α-carbon of another residue. A specific example is AlbA (NP_391617).

Specifically, cytolysin forming enzymes catalyse the formation of a thioether bond in the where the stereochemistry at the α and β carbons originating from Thr is inverted relative to other thioether within the peptide. Specific example is CyIM (AAA62650).

Cyanobactin Forming Enzymes:

Cyanobactins are small cyclic peptides that are produced by a diverse selection of cyanobacteria. Cyanobactins are produced through the proteolytic cleavage and cyclisation of precursor peptides coupled with further posttranslational modifications such as heterocyclisation, oxidation, or prenylation of amino acids.

Thiopeptide Forming Enzymes:

A defining feature of the thiopeptide macrocycle is a six-membered nitrogenous ring that can be present in one of three oxidation states: a piperidine, dehydropiperidine, or pyridine. Further architectural complexity is achieved in some thiopeptides by the addition of a second macrocycle to incorporate a tryptophan-derived quinaldic acid or an indolic acid residue, such as those found in thiostrepton A and nosiheptide.

Specifically, conopeptide forming enzymes catalyse the C-terminal amidation of glycine to form C-terminal —CO—NH2, N-terminal cyclisation of glutamine to form N-terminal pyroglutamate, hydroxylation of Pro, Val and Lys to form 4-hydroxyproline, D-γ hydroxyvaline and 5-hyroxylysine respectively, carboxylation of glutamate to form γ-carboxyglutamate, sulphation of tyrosine to form sulphotyrosine, bromination of tryptophan to form 6-bromotryptophan, oxidation of cysteine to form disulphide bridges, glycosylation of threonine to form various O-glycosyl amino acids, epimerization of Val, Leu, Phe and Trp to form D-valine, D-leucine, D-phenylalanine and D-tryptophan respectively.

Microviridin Forming Enzymes:

Microviridins represent a family of cyclic N-acetylated trideca and tetradecapeptides that contain intramolecular ω-ester and ω-amide bonds.

Bacteriocin Forming Enzymes:

Historically, ribosomally synthesized and post-translationally modified peptides have been subdivided based on either the producing organisms (e.g. microcins produced by Gram-negative bacteria) or their biological activities (e.g. bacteriocins). Bacteriocins are peptides that inhibit to growth of related bacterial strains.

Sulphur chemistry converts the thiols of cysteines to disulfides (cyclotides, conopeptides, lanthipeptides, cyanobactins, lasso peptides, sactipeptides, and glycocins), thioethers (lanthipeptides, sactipeptides, phalloidins, some thiopeptides), thiazol(in)es (thio-peptides, LAPs, cyanobactins, bottromycins), and sulfoxides (Ian-thipeptides, amatoxins).

Different classes of PTME may be used. While the bifunctional PTME, like ProcM are considered a LanM-type enzyme (class II), further PTME of different classes may be used in addition to the bifunctional dehydratase and cyclase.

A specific combination of PTME of different classes is the following: a lanthionine-bond forming enzyme and a microviridine forming (ester bond and amide bond forming) enzyme, e.g. ProcM combined with MvdD and MvdC.

Further description of the enzymes is provided below.

Specifically lanthionine bond forming enzymes are employed. Lantibiotics (Willey and van der Donk Annu. Rev. Microbiol. 2007. 61:477-501) are defined as lanthionine-containing antibiotics. Intramolecular bridges are termed lanthionine or methyllanthionine bonds, which are arising from the posttranslational modification of different amino acid side chains. Serine and threonine hydroxyl groups are dehydrated to yield 2,3-didehydroalanine (Dha) or (Z)-2,3-didehydrobutyrine (Dhb), respectively. This is followed by the stereospecific intramolecular addition of a cysteine residue onto Dha or Dhb to form the lanthionine or methyllantionine bond. Currently several posttranslationally modifying enzymes and their genes are known: LanB type dehydratases are shown to constitute the C-terminus of the enzyme proposed to catalyse the dehydration step of serine and threonine. LanC type cyclases catalyses the addition of cysteine thiols. LanC, the cyclase component, is a zinc metalloprotein, whose bound metal has been proposed to activate the thiol substrate for nucleophilic addition. LanM type fused dehydratases and cyclases: It is responsible for both the dehydration and the cyclisation of the precursor-peptide during lantibiotic synthesis.

Specific examples of LanM type enzymes are ProcM and functional variants, and homologs, as further described herein.

LanD oxidative decarboxylase type enzymes: This enzyme catalyses the removal of two reducing equivalents from the cysteine residue of the C-terminal meso-lanthionine of epidermin to form a —C══C— double bond. LanP type peptidases cleave the leader peptide from the lantibiotics. A LanT type peptidase is fused to an ABC transporter; the cleavage of the precursor peptide is mediated by the transporter as part of the secretion process. LtnM and LtnJ type of dehydratase and dehydratase are involved in the formation of D-alanine. CinX hydroxylates asparagines during cinnamycin biosynthesis.

Microcins: (Duquesne et al Nat Prod Rep. 2007 August; 24(4):708-34) are mostly produced by enterobacteria and classified in three groups: Class I, IIa and IIb.

Involved PTME and their genes are: McbB like serine and cysteine dehydratases (cyclodehyratases), McbC like flavine dependent dehydratase (oxidoreductase), TIdE protease involved in proteolytic processing of the antibiotic Microcin B17, PmbA (TIdD) microcin processing peptidase 2 type, MccB type modification enzyme involved in Microcin MccC7/C51 biosynthesis, MccD type transfer of n-aminopropanol groups (MccC7/C51), McjB and McjC involved in Microcin J25 processing and maturation, MceC type glycosyltransferase involved in Microcin E492 modification, MceD enterobactin esterase, MceI acetyltransferase.

The further group of cytolysin forming enzymes: Streptolysins (Mitchell et al, J Biol Chem. 2009 May 8; 284(19):13004-12) are posttranslationally modified peptides from Clostridium botulinum. Specific PTM enzymes are: SagB a dehydratase and SagC a serine and cysteine dehydratases (cyclodehydratase). Both enzymes are involved in the formation of thiazole and (methyl)-oxazole formation.

Cyanobactins (Schmidt et al Proc Natl Acad Sci USA. 2005 May 17; 102(20):7315-20) are cyclic peptides containing heterocycles isolated from different cyanobacteria) genera. According to a preferred embodiment cyanobactin forming enzymes may be used such as PatA a subtuilisin like serine protease peptidase which cleaves the precursor peptide, PatD a serine and cysteine dehydratases (cyclodehyratases) and PatG a dehydratase (oxidoreductase).

Thiopeptides (Morris et al J Am Chem Soc. 2009 Apr. 29; 131(16): 5946-552009) are a class of heterocycle-containing posttranslationally modified peptides which have a characteristic tri- and tetrasubstituted pyridine ring at the junction of the macrocycle. Involved PTM enzymes are TpdB dehydratase involved in the pyridine ring formation.

TpdC dehydratase is involved in the pyridine ring formation, TpdG cysteine dehydratase (cycloydehyratase) involved in thiazoline, TpdE dehydratase (oxidoreductase) converts thiazoline to thiazole formation, TpdH peptidase, Tpdl radical SAM protein, coproporphyrinogen III oxidase, TpdJ1 P450 monooxygenase, TpdJ2 P450 monooxygenase, TpdL radical SAM is protein involved in the C-methylation, TpdM O-methyltransferase, TpdN deamine reductase, TpdO cyclodehydratase, TpdP dehydratase, TpdQ P450 Monooxygenase, TpdT N-methyltransferase and TpdU radical SAM protein.

Conopeptide forming enzymes: Conopeptides (Buczek et al Cell. Mol. Life Sci. 62 (2005) 3067-3079) are a class of postranslationally modified peptides produced by cone snails. It is estimated that the class of conopeptides constitute a group of 100.000 different peptides. Specific PTM enzymes are Tex31, a substrate-specific endoprotease, MrPDI a specific protein disulfide isomerase, and a vitamin K-dependent carboxylase (Accno: AF382823). From biochemical experiments it could be shown that the following enzyme reactions are involved in conotoxin formation: a proline, valine, lysine hydroxylation, a protein amidating reaction by a specific monooxygenase, the tryptophan bromination to 6-bromotryptophan by a specific bromo peroxidase, and epimerization of Trp, Leu, Phe, Val by an epimerase. Further there are a glutaminyl cyclase, a tyrosyl sulfo transferase, and an O-glycosyltransferase involved in conotoxin biosynthesis.

Amatoxins/Phallotoxins (Walton et. al., Biopolymers. 2010; 94(5):659-64. doi: 10.1002/bip.21416, Hallen et al Proc Natl Acad Sci USA. 2007 Nov. 27; 104(48):19097-101) are posttranslationally modified peptides isolated from Amanita basidiomycetes.

A specifically known PTME is Pop1, a serine protease. From biochemical experiments it is deduced that a cyclase, a hydroxylase, and an enzyme involved in tryptophan-cysteine tryptathione cross linking is involved in the formation of amatoxin/phallotoxin production.

Microviridin forming enzymes: Microviridins (Philmus et al., 2008 see above, Ziemert et al., Angew Chem Int Ed Engl. 2008; 47(40):7756-9): Microviridins are a class of tricyclic peptides, which have been isolated from different cyanobacterial genera. The PTME involved in the maturation of the Microviridins have been biochemically characterized. Specific PTME are MvdB an acetyltransferase, MvdC a cyclisation protein involved in the amide bond formation, similar to RimK ATP-binding proteins, ATP grasp ligase, MvdD cyclisation protein involved in the formation of the two ester bonds, similar to RimK ATP-binding proteins and ATP grasp ligases.

Cyclotides (Saska et al J Biol Chem. 2007 Oct. 5; 282(40):29721-8) are a group of posttransaltionally modified peptides isolated from plants of the Violaceae, Rubiaceae and Curcurbitaceae families. Among the characterized PTM enzymes are two peptidases involved in cyclotide formation: NbVpe1a and NbVpe1b.

Circular bacteriocins (Maqueda et al FEMS Microbiol Rev. 2008 January; 32(1):2-22) belong to a group of posttranslationally modified peptides isolated from Gram-positive bacteria. Although the gene operons which are coding for the enzymes responsible for the formation of circular (cyclic) bacteriocins have been isolated and described, the identification of biochemical steps catalyzed by the candidate enzymes has not been completed. From biochemical experiments it has been deduced that a peptidase, which catalyses a head-to tail circularization is involved in circular bacteriocin formation. The epimerization of an L-alanine to a D-alanine is catalyzed by a specific epimerase. Also preferred are subtilosin forming enzymes, like AlbA a Fe—S Oxidoreductase, AlbF a Zn-dependent peptidase and AlbE a second Zn-dependent peptidase.

Subtilosin forming enzymes: AlbA AP011541.1Fe—S Oxidoreductase, AlbF AP011541.1Zn-dependent peptidase and AlbE AP011541.1Zn-dependent peptidase.

Among the further PTME described herein are proteases that cleave the cyclic peptide product from a signal and/or leader peptide. According to a specific embodiment, the PTME modifies a substrate peptide that is fused to a PTME-leader sequence which is at least partly responsible for recognition by the modifying enzymes and/or by export machinery. However, the leader may as well be provided as a separate entity, such as an additive or by co-expression employing a co-expression vector that contains the leader sequence. The substrate peptide and the leader may then be co-expressed in the same recombinant host and the peptide is post-translationally modified. Alternatively, the leader and the PTME may be provided as an additive to the recombinant host cell culture, which host is then capable of expressing the mature cyclic peptide. Specific PTME modify cysteine, serine and threonine residues, others modify carboxyl groups of asparagine or glutamine, e.g. to form heterocyclic moieties, such as thiazole and oxazole moieties by post-translational processing. Therefore the PTME may act as a single protein or a single-subunit enzyme, as well as a protein-enzyme complex comprising at least two, three or four different enzymes to support the heterocyclisation. Specifically, genes or gene clusters involved in the biosynthesis of heterocyclic ribosomal peptides, including functionally active variants, may be employed in accordance with the present invention.

The term “PTME” as used herein shall include the naturally-occurring enzyme, e.g. derived from an origin without modification by sequencing the original molecule and recombinant production of the PTME, or by using a cell lysate of a naturally occurring cell producing the original PTME, optionally upon purification. The term shall further include the functionally active variants of the PTME. Natural enzymes may be preferably used, e.g. such as from lysates of organisms. Specifically, enzymes of bacterial origin are used, such as derived from a bacterial lysate. Alternatively, recombinant enzymes may be used, which have the same sequence as the wild-type (naturally-occurring) enzyme, or enzyme variants which are functionally active, preferably comprising the catalytic region or fragment of a naturally-occurring enzyme, e.g. to engineer a variant with altered substrate specificity or enzymatic activity.

Specifically, the PTME termed “ProcM” is understood as the naturally occurring enzyme of Prochlorococcus marinus or Synechococcus sp. origin, such as ProcM9313 originating from Prochlorococcus marinus which is specifically characterized by its amino acid sequence SEQ ID 1, ProcM9303 originating from Prochlorococcus marinus which is specifically characterized by its amino acid sequence SEQ ID 2, and ProcM9916 originating from Synechococcus sp. which is specifically characterized by its amino acid sequence SEQ ID 3, and functionally active variants of any of the foregoing, e.g. comprising at least one functional catalytic site identified by the amino acid sequence SEQ ID 4, 6, 8 (dehydratase activity) or SEQ ID 5, 7, 9 (cyclase activity), or the combination of one of the SEQ ID 4, 6, 8 with one of the SEQ ID 5, 7, 9 to obtain the bifunctional molecule comprising two active sites, of the same or different origin, including functionally active variant comprising a modified catalytic site with functional activity.

The term “leader” or “PTME-leader peptide” as used herein shall refer to a peptide comprising at least a recognition motif for a PTME. Substrate specificity of a PTME is typically determined by the presence of the cognate PTME-leader as a co-substrate in close proximity to a substrate peptide that contains amino acid residues to be modified to form bridge piers of a heterocycle.

The co-substrate function of the PTME-leader is understood in the following way: The PTME is capable of recognizing a peptide as a substrate in the presence of the PTME-leader, i.e. the co-substrate. Thus, the PMTE-leader is a co-factor that turns an arbitrary peptide into a substrate, provide that the arbitrary peptide comprises target amino acid residues that are capable of being a bridge pier of a heteroatom bridge. While it is understood that the PMTE-leader is a separate entity and differentiated from a core/substrate peptide, the PTME-leader itself may be subject to enzymatic transformation, if it is placed in conjunction (or fused to) the core peptide.

According to a preferred aspect the PTME-leader peptide is an integrated leader sequence on the genetic package, e.g. N- or C-terminally fused to a substrate peptide, so to provide a preceding (leading) sequence or a succeeding (following) sequence, or the leader may be provided as a separate entity, e.g. as a separate peptide separate from or independent of the substrate peptide or displayed by the same genetic package aside from the position where the substrate peptide is displayed, e.g. through fusion to different anchor proteins, or provided independent of the genetic package displaying the substrate peptide, such as an additive or by means of a helper display system displaying a leader sequence or a variety of leader sequence mutants to act in support of the PTME. According to a specific embodiment, the leader is provided in conjunction with the PTME, e.g. as an ELF.

The term “substrate peptide” as used herein shall refer to a peptide including elements supporting the post-translational enzymatic processing (maturation) of the peptide. The substrate peptide sometimes called “core” peptide is recognized by a PTME in the presence of a PTME-leader, e.g. providing a precursor element, i.e. a signal and/or leader sequence, operationally linked to a core peptide. In a PTME-leader fused to a core peptide resulting in a fusion peptide, typically the core sequence is enzyme-modified. Precursor elements or extensions are usually cleaved from the mature core peptide following modification (e.g. upon cyclisation), resulting in a short peptide product.

For enzymatically processing of naturally-occurring ribosomal peptides by wild-type organisms, different segments of a precursor peptide are as follows: Signal peptides direct the transport to specific subcellular compartments; signal peptides are recognition motifs for cellular proteins. The core peptide (herein understood as substrate peptide) is a segment which is to be processed to become a mature secondary metabolite, and in some examples, the cyclisation recognition sequence is at the C-terminus following the core peptide segment. Generally there are five steps involved in the production of a secondary metabolite. First the messenger mRNA coding for the precursor peptide is translated using ribosomes and transport tRNAs. The precursor peptide, which can be consisting of the above mentioned segments, is then recognized by dedicated PTME which post-translationally modify the core segment resulting in molecular crosslinks leading to a cyclic architecture. A protease cleaves off the leader peptide and thus separates the leader peptide from the core peptide segments. Finally, a transport protein moves the mature secondary metabolite over a membrane. Specific mutations blocking the cleavage of the leader may be advantageous for displaying a precursor peptide according to the invention.

According to a different mechanism, translocation may as well precede the cyclisation.

When a display system displaying the substrate peptide is used, it is preferred that the cyclised peptide still includes a precursor element, such as the signal or leader sequence, even after maturation of the core peptide into the cyclic peptide. Thus, the preferred display system or construct is engineered to block the cleavage of the core peptide either before and/or after the maturation process. This may be effected by a mutation to prevent cleavage or through establishing suitable bridges crossing the cleavage site.

When a bifunctional PTME and/or a combination of at least two PTME is used, the enzymatic reactions may occur as a series of reactions in a predefined order, thus, the substrate peptide for a first enzymatic reaction differs from the substrate peptide for a second enzymatic reaction in at least one of the amino acid sequence, e.g. including one or more dehydrated amino acid residues, or the cyclic structure. Alternatively, the enzymes may recognize the substrate peptide irrespective of prior enzymatic modification, thus, a more heterogeneous variety of cyclic peptides may be produced.

The substrate peptide may be a naturally-occurring ribosomal peptide, herein also referred to as a ribosomal peptide, e.g. originating from a wild-type organism, or a functionally active variant thereof, such as comprising hypervariable or randomised sequences, or a fully randomised peptide or artificial peptide sequence, including peptides of rational design.

The term “ribosomal peptide” as described herein shall mean biologically active, ribosomally synthesized peptides of structural diversity, most commonly up to about 100 amino acids long, which are post-translationally modified by various enzymes that catalyze the formation of a large number of different chemical motifs. Within this class, there are numerous substrate peptides with hypervariable sequences. The primary peptides can act as substrates for the processing pathways, and so each pathway leads to numerous different mature peptides. Members of this class have a high potential possibly important in microbiology, the environment, medicine and technology. Commonly, the substrate peptide which is a natural ribosomal peptide is combined with or may contain a relatively conserved leader sequence that is at least partly responsible for recognition by the modifying enzymes and/or by export machinery. These biosynthetic mechanisms are nearly universal for the bacterial ribosomal peptide natural products and are also commonly found in the biosynthesis of similar peptides from other organisms, such as archea, fungi, plants and animals.

Specific ribosomal peptides as described herein include microviridins, lacticins, thiopeptides, conopeptides, microcins, cytolysins, lantibiotics, cyanobactins, amatoxins/phallotoxins, cyclotides and (cyclic) bacteriocins, which are naturally-occurring or or functionally active variants thereof.

According to the invention it is possible for the first time to provide for complex metabolic processing of substrate peptides, such as ribosomal peptides or functionally active variants or synthetic substrate peptides, in particular by using a bifunctional PTME, thereby forming a cyclic peptide comprising a thioether bridge which is displayed by a displaying genetic package system.

It turned out that the modifying processes of metabolic, enzymatic processing can be applied to genetic packages displaying substrate peptides and to peptide libraries in general. Bifunctional PTME, such as ProcM, which have both, a dehydratase and cyclase activity, were used in the prior art only with respect to enzymatic conversion in solution (in vitro). There was no indication that such bifunctional ProcM enzyme could be successfully used to produce cyclic peptides immobilised by a displaying genetic package.

The present invention particularly relates to genetic packages that display a cyclic peptide and methods of producing such packages, e.g. comprising

a) providing a nucleic acid sequence encoding a substrate peptide comprising a specific number of Ser/Thr and Cys, preferably at least one Ser or Thr and at least one Cys;

b) ligating said nucleic acid sequence into the genome of a genetic package;

c) displaying the corresponding peptide sequence by the genetic package; and

d) enzymatically processing the substrate peptide with a bifunctional PTME, which has dehydratase and cyclase activity, to produce a mature cyclic peptide comprising a thioether bridge connecting the Ser/Thr and Cys residues, which is displayed by the genetic package.

Process steps c) and d) may be carried out in a consecutive way, or else in a single process step, so that the enzymatic processing occurs while the peptide is expressed to be displayed by the genetic package. Thus, the enzymatic processing may be carried out in situ, while translating, transporting and displaying the peptide, so that the mature cyclic peptide is obtained in the immobilised form.

The genetic package as used according to the invention preferably is provided as a member of a library, which library members display a diversity of peptides, also called peptide library. Thus, the present invention also provides for a process of preparing a respective peptide library comprising a repertoire of genetic packages displaying a variety of cyclic peptide structures.

A specific method of producing such a library comprises

a) providing a repertoire of nucleic acid sequences encoding substrate peptide variants, wherein each of the variants comprise a specific number of Ser/Thr and Cys, preferably at least one Ser or Thr and at least one Cys;

b) ligating said repertoire into the genome of genetic packages;

c) displaying the corresponding peptide sequences by said genetic packages to obtain a peptide library, and

d) enzymatically processing said peptide library with a bifunctional PTME, which has dehydratase and cyclase activity, to produce a library of mature cyclic peptides that is displayed by the genetic packages, which library comprises a variety of peptides, each comprising a thioether bridge connecting the Ser/Thr and Cys residues, which differ in the cyclic structure, which is displayed by the genetic package.

Again, process steps c) and d) may be carried out in a consecutive way, or else in a single process step, so that the enzymatic processing occurs while the peptides are expressed to be displayed by the genetic packages. Thus, the enzymatic processing may be carried out in situ, while translating, transporting and displaying the peptides, so that the mature cyclic peptides are obtained in the immobilised form.

Therefore a repertoire of nucleic acid sequences encoding the variety of cyclic peptides can be ligated into the gene of the replicable genetic package.

To anchor the peptide to a filamentous bacteriophage surface, genetic fusions to phage coat proteins can be employed. Preferred are fusions to gene III, gene VIII, gene VI, gene VII and gene IX, and fragments thereof. Furthermore, phage display has also been achieved on phage lambda. In that case, gene V protein, gene J protein and gene D protein are well suitable for the purpose of the invention. Besides using genetic fusions, foreign peptides or proteins have been attached to phage surfaces via association domains, including a tag displayed on phage and a tag binding ligand fused to the peptide to be displayed to achieve a noncovalent display, but also display systems including connector compounds for covalent display.

Natural ribosomal peptides are preferably used as a scaffold to prepare a repertoire of variants with different modifications at specific sites. Variants of a parent structure, such as the ribosomal peptide scaffold, are preferably grouped to form peptide libraries, which can be used for selecting members of the library with predetermined functions. In accordance therewith, a scaffold sequence is preferably randomised, e.g. through mutagenesis methods. According to preferred strategies specific positions within the peptide sequence are mutated, which provide for new bridge piers of heteroatom bridges of the heterocycle. Alternatively the mutated positions are aside from the existing bridge piers, so to generate diversity while maintaining the cyclic peptide structure.

According to a preferred embodiment a loop region or terminal region of a substrate peptide sequence comprising positions within one or more loops or at a terminal site, potentially contributing to a target binding site, is preferably mutated or modified to produce libraries. Mutagenesis methods preferably employ random, semi-random or, in particular, by site-directed random mutagenesis, thus, resulting in a randomised sequence, in particular to delete, exchange or introduce randomly generated inserts. Alternatively preferred is the use of combinatorial approaches. Any of the known mutagenesis methods may be employed, among them cassette mutagenesis. In some cases positions and amino acids are chosen randomly, e.g. with any of the possible amino acids or a selection of preferred amino acids to randomise a sequence, or amino acid changes are made using simplistic rules. For example all residues may be mutated preferably to specific amino acids, such as alanine, referred to as amino acid or alanine scanning. Such methods may be coupled with more sophisticated engineering approaches that employ selection methods to screen higher levels of sequence diversity.

Any kind of prior art peptide library may be subject to metabolic processing and maturation employing the PTME according to the invention, because random peptide libraries typically contain a variety of peptides that qualify as a substrate, i.e. which comprise a specific number of Ser or Thr and Cys residues. Thereby the peptide library can be improved through three-dimensional, constrained structures of the peptides. This increases the chance for high affinity and/or high specificity binders.

Usually peptide libraries according to the invention comprise at least 10⁶ library members, more preferred at least 10⁷, more preferred at least 10⁸, more preferred at least 10⁹, more preferred at least 10¹⁰, more preferred at least 10¹¹, up to 10¹², even higher number are feasible.

To avoid experimental limitation in the construction of a complete library, either a restricted set of nucleotides/amino acids or the complete set of nucleotides/amino acids with an increased proportion of specific ones is preferably used for randomisation or randomisation within a structural scaffold may be used. Loop structures typically play an important role in the molecular recognition of protein—protein or protein—peptide interactions. Specific library designs provide for randomisation besides one or more of predetermined bridge piers of enzymatic cyclisation.

Prochlorosin libraries are based on known Prochlorosins found in nature where the existing bridging residues remain unmutated. The dehydrated residues, which are not involved in bridge formation may be specifically mutated. For example, the N-terminus is randomly mutated, including the increasing or decreasing the number of residues before the first bridging residue extending the N-terminus between zero and 100 residues. The residues in the loops are randomly mutated including the increasing or decreasing the number of residues in each loop either individually for each loop or in combination in two or three of the loops. A C-terminal extension of random amino acid residues of between zero and 100 may be added. A C-terminal extension which deliberately includes cysteine residues at each position can be added to allow for extra ring formation with the dehydrated residue in the N-terminus or with residues that have been introduced as part of the randomisation process.

The following libraries can be made based on Prochlorosin 1.7

TIGGTIVSITCETCDLLVGKMC (SEQ ID 98)

where the residues are randomised producing the following sequence

TXaa₍ₙ₌₃₎TXaa₍ₙ₌₂₎SXaaTCXaaTCXaa₍ₙ₌₇₎C (SEQ ID 99)

wherein Xaa is any of the 20 naturally-occurring amino acids.

TXaa₍ₙ₎TXaa₍ₙ₎SXaa₍ₙ₎TCXaa₍ₙ₎TCXaa₍ₙ₎CXaa₍ₙ₎ (SEQ ID 100)

wherein Xaa is any of the 20 naturally-occurring amino acids; and n=1-100.

A library based on Prochlorosin 2.8

AACHNHAPSMPPSYWEGEC (SEQ ID 101), which has two non-overlapping rings, can be made with the following features

Xaa₍ₙ₌₀₋₁₀₀₎CXaa_{(N=1-10)}SXaa_{(N0-10)}SXaa_{(N1-10)}CXaa₍ₙ₌₀₋₁₀₀₎(SEQ ID 102)

wherein X is any of the 20 naturally-occurring amino acids.

Conventional peptide libraries, which are libraries that are not derived from naturally-occurring ribosomal peptides as scaffold i.e. random libraries can be constructed using codons encoded by oligonucleotides where the codon is synthesised as NNN, NNS, or preferably NNB. To further increase the chances of obtaining a peptide with serine or threonine and cysteine residues which can be involved in the bridge formation a doped (focussed) library is made where the proportion of each base is varied to bias the codon towards coding for the desired residues. In this library N at the first position of each codon (synthesized with an equal proportion of each base), N at the second position of each codon where

C=30%, and T/A/G=23.3%;

C=40%, and T/A/G=20%; or

C=50%, and T/A/G=16.7%;

or an even higher proportion of C may be used (up to 90%) with the corresponding decrease in the other bases: According to an alternative embodiment K at the third (T55%, G45%) or T60% or T65% etc. with the corresponding decrease in G may be used.

Exemplary libraries for phage display may be designed so that the signal peptidase that normally cleaved the signal sequence from the N-terminus of PIII of phage generated the correct N-terminus for the desired peptide. The core peptide is cloned between the signal sequence and the beginning of domain 1 of gene 3 in a phage or phagemid vector. Mutagenesis is the preferred method for library generation as it removes the need for engineering in restriction sites for cloning.

Alternatively, full or partial random peptide sequences are used. The full random sequences may contain a bias towards particular bases at each position in each codon to increase the likelihood of obtaining the correct residues for bridge formation. Partly random libraries may contain particular residues at particular places to increase the likelihood that bridges can be formed. For instance, a library that is to be used with ProcM may have cysteine and/or serine and/or threonine residues deliberately added at particular positions.

An alternative is where the oligonucleotides used for the mutagenesis are synthesised using trinucleotide phosphoramitides where the proportion of each trinucleotide has been adjusted to achieve the desired level of serine or threonine and cysteine residues.

An alternative is where the library is made with the deliberate inclusion of a single or double cysteine residue at or near the N- and C-termini or at both termini of the peptide, as is seen in Prochlorosins 1.1, 2.1, 2.2, 2.4 and others. The rest of the residues are encoded randomly, which with the natural prevalence of codons that encode for serine and threonine creates library members with the correct number of residues for bridge formation.

The length of the peptides of random libraries is typically between 10 and 40 residues.

An exemplary library may also be made with a bias towards basic residues, which are often found in cell penetrating peptides. The bias is generated by increasing the proportion of the bases A and C at the first position of each codon and the proportion of A and G in the second position or each codon, when using NNS codons for randomised positions.

Another exemplary library may also be made with a bias towards hydrophobic residues as this can help generate peptides with cell penetrating properties and oral bioavailability. This library is made by increasing the proportion of G in the first position of each randomised codon and increasing the proportion of T whilst decreasing the proportion of A in the second position of each codon.

The library may be constructed so that there is a spacer or linker between the library and D1 of PIII of the phage. The linker can be peptidic and of a length between 0 and 20 residues. The linker increases the distance between the peptide being modified and D1 of PIII thereby minimising steric hindrance of the modifying enzyme. An alternative is to add a protein between the linker and the phage. This aids subcloning of the hits from selections and can help with expression of the peptides after subcloning.

The libraries may be constructed using mutagenesis of a phage vector containing the leader sequence for a ribosomal peptide or a related family member downstream and adjacent to the signal sequence for PIII in phage. The phage vector may be mutated to include restriction enzyme recognition site for the cloning in of the library and/or the leader sequence plus the library and the subcloning of hit sequences. The leader sequence may also be altered to allow for the cleavage between the leader and the core peptide by a commercially available protease after the cyclisation reaction.

Alternatively, the vector can be mutated to include the PTME-leader sequence and the library is then mutated into the vector.

Alternatively, the substrate (core) peptide may be cloned into a phage vector between the phage signal sequence and the leader peptide. This reverse orientation uses the leader peptide as a spacer for the core peptide and may allow for transactivation of the modifying enzyme.

Alternatively, the core peptide library may be mutated into the phage vector directly after the PIII signal sequence for where the leader sequence peptide is added to the modification buffer.

Alternatively, the core peptide library may be mutated into the phage vector directly after the PIII signal sequence in a phage vector where the leader sequence is fused to P6 of the phage. The co-display of the leader and the core peptide on the same phage enables transactivation of the modifying enzyme. The display of peptides on P6 of phage is C-terminal. The orientation of the leader peptide by the C-terminal display is favourable for a bipartite display of the leader and core peptide as it orientates the enzyme so as to avoid steric hindrance to the access of the core peptide by the phage.

Alternatively, a phagemid system may be used. For example, the core peptide is directly after the phage signal sequence of gene 3. A helper phage is used which has the leader sequence for the peptide cloned between the phage signal sequence and the start of domain 1 of gene 3. The co-display of the leader and the core peptide on the same phage enables transactivation of the modifying enzyme.

The library or repertoire of peptides typically is combined with one or more PTME under conditions suitable for enzymatic activity. Conditions that are suitable for the enzymatic activity of PTME are well-known in the art or can be readily determined by a person of ordinary skill in the art. If desired, suitable conditions can be identified or optimized, for example, by assessing the enzymatic activity under a range of pH conditions, enzyme concentrations, temperatures and/or by varying the amount of time the library or repertoire and the enzyme are permitted to react.

For example, a single enzyme, any desired combination of different enzymes, or any biological preparation, biological extract, or biological homogenate that contains enzymatic activity can be used. Suitable biological extracts, homogenates and preparations that contain enzymatic activity include extracts with aqueous organic solvents, lysates and the like.

For example the bifunctional PTME as described herein may be combined with any of the following:

MvdB (accession number: ACC54548), MvdC (accession number: ACC54549) and MvdD (accession number: ACC54550) or their homologues like MdnB (accession number CAQ16122, CAZ67054) and MdnC (accession number CAQ16123, CAZ67055).

In particular, a combination of MvdD and MvdC, or a combination of MdnB and MdnC, or a combination of MvdD and MdnC, or a combination of MvdC and MdnB may be used.

Any of MvdD and MvdC would be used with a cognate leader, e.g. the MvdE-Leader.

Any of MdnB or MdnC would be used with a cognate leader, e.g. the MdnA-Leader.

Upon metabolic processing the library members with the appropriate peptide structures may be analysed. It may be preferred to amplify or increase the copy number of the nucleic acids that encode the selected peptides to obtain sufficient quantities of nucleic acids or peptides for additional rounds of selection or for preparing additional repertoires, e.g. for further enzymatic processing to further specific randomisation, e.g. for refining the specificity or affinity maturation purposes. For example, phage amplification, cell growth or PCR techniques may be employed. In a preferred embodiment, the display system is bacteriophage display and the amplification is through expression in E. coli.

A target binding peptide can be selected from a peptide library according to the invention using a desired binding or biological activity selection method, which allows peptides that have the desired activity to be distinguished from and selected over peptides that do not have the desired activity. Generally, one or more selection rounds are required to separate the replicable genetic package of interest from the large excess of non-binding packages. For example, peptides that bind a target ligand can be selected and recovered by panning. Panning may be accomplished by techniques well-known in the art. Suitable assays for peptide activity can be used to select the library members for further characterization. For example, a common target binding function can be assessed using a suitable binding assay, e.g. ELISA.

Preferably screening a phage-displayed peptide library is accomplished through an affinity-selection process referred to as panning or biopanning. Biopanning typically comprises incubating the peptide library with the target, washing away unbound phage, eluting the remaining bound phage, and amplifying the eluted phage for subsequent screening rounds. After multiple rounds of biopanning, the target-binding phage may be enriched and individual phage are isolated and sequenced to reveal any enriched binding motif.

When a phage display system is used, binding is preferably tested in a phage ELISA. Phage ELISA may be performed according to any suitable procedure. In one example, populations of phage produced at each round of selection can be screened for binding by ELISA to the selected target to identify phage that display target binding peptides. According to a commonly used procedure soluble peptides may be tested for binding to the ligand, for example by ELISA using reagents, for example, against a C- or N-terminal tag. The diversity of the selected phage may also be assessed by gel electrophoresis of PCR products or by sequencing of the vector DNA.

Depending on the target application specific peptides can also be selected based on catalytic or enzyme inhibitory activity, which can be measured using an enzyme activity assay. Further biological tests for screening suitable peptides are based on the desired antibiotic, antifungal, or otherwise bioactivity, such as inhibitor or cofactoractivity, e.g. enzyme inhibitor or enhancer activity employing the suitable cell based assays.

Suitable ligand targets are preferably selected from structures or epitopes of microbes, such as bacterial, fungal, parasitic or viral, but also of human or animal or plant or insect cells, including proteins, specifically enzymes, co-factors for enzymes, receptors, growth factors, DNA binding proteins, nucleic acids, lipids and carbohydrates.

Binding peptides or the DNA encoding the peptides can be isolated from the replicable genetic package and characterised. Depending on the application form, the lead peptide may then be synthesized or combined with standard molecular biological techniques to make constructs encoding peptide fusions. Suitable methods of preparing the peptides or peptide fusion constructs e.g. employ recombinant expression techniques, such as expression by recombinant bacterial or yeast cells.

The peptides identified and provided according to the invention may serve as leads for development into therapeutics or diagnostic reagent, or may be manipulated to target a unique molecular entity for specific and discriminatory drug delivery. Particularly preferred applications are in the field of mimotopes of biological targets, e.g. for use as an inhibitor, such as antibacterial, antifungal, antiparasitic, antiviral, enzyme inhibitors and antibiotics, or for developing a vaccine. Further applications are feasible for industrial, agricultural, pesticidal use (including agents for plant protection or pesticides), food or feed use (including food or feed additives or food preservatives), analytical or environmental applications, which employ a target binding moiety.

A pharmaceutical composition comprising the peptide obtained according to the invention typically further comprises at least one pharmaceutically acceptable excipient well known to the skilled person. The pharmaceutical composition may further comprise at least one other biologically active agent. Suitable agents are also well-known to the skilled artisan. A preferred peptide composition as obtained according to the invention may comprise stabilising molecules, such as albumin or polyethylene glycol, or salts. Preferably, the additives used are those that retain the desired biological activity of the peptide and do not impart any undesired toxicological effects.

The examples described herein are illustrative of the present invention and are not intended to be limitations thereon. Different embodiments of the present invention have been described according to the present invention. Many modifications and variations may be made to the techniques described and illustrated herein without departing from the spirit and scope of the invention. Accordingly, it should be understood that the examples are illustrative only and are not limiting upon the scope of the invention.

### EXAMPLES

### Example 1: Production of a ProcM Library

The library is constructed using mutagenesis using the method of Sidhu et al (Methods Enzymol, 2000; 328: 333-363). After the mutagenesis the library DNA is purified using Qiagen PCR purification kit, or similar, eluting into pure water or 10 mM Tris pH 8.0. The DNA is mixed with E. coli TG1 strain (BioCat GmbH) that has been prepared to be electrocompetent. Electroporation is performed using a BioRad electroporator using an appropriate setting for E. coli using either 1 mm or 2 mm gap electroporation cuvettes. Immediately after electroporation 1 ml of SOB media at 37° C. is added to the cuvette and the liquid transferred to a 250 ml Erlenmeyer flask. A second 1 ml of SOB is used to wash the cuvette and this liquid is also added to the Erlenmeyer flask. After further electroporations, if required, the volume in the flask is made up to 25 ml with SOB media. The flask is placed at 37° C. with shaking. After 1 hour, a sample of the culture is taken titre the number of transformants by plating dilutions of the culture on LB+tetracycline agar plates. The plates are kept at 37° C. overnight and the colonies are counted in the morning. From this the size of the library is calculated. The remainder of the 25 ml culture is added to one to four 21 Erlenmeyer flasks containing 500 ml of 2TY+tetracycline media. This is grown overnight at 37° C. with shaking. The cells are removed by centrifugation, 10000 rpm in a Beckman JA-10 rotor for 25 minutes at 4° C., and the supernatant has ⅕^{th} volume of PEG 8000 (20% W/V)/NaCl(2.5 M) solution added and placed on ice for 30 minutes to precipitate the phage. The precipitated phage are harvested by centrifugation, 10000 rpm in a Beckman JA-10 rotor for 10 minutes at 4° C., resuspended in 20 ml of 10 mM Tris pH 8.0/0.1 mM EDTA and recentrifuged, 9400 g for 10 minutes at room temperature, to remove any remaining bacteria. The resultant phage solution is subjected to a CsCl gradient centrifugation by making the volume up to 28 ml with 10 mM Tris pH 8.0/0.1 mM EDTA and adding 12.8 g of CsCl. This is mixed until the CsCl had dissolved. The solution is divided between 4 centrifuge tubes and then this is spun at 48 000 rpm in an S80-AT3 rotor in a Sorvall MTX 150 Micro-Ultracentrifuge for between 20 and 96 hours until a band of phage is visible. The phage band is harvested and the phage solution is injected into a 10KDa cut off dialysis cassette and dialysed at 4° C. into 10 mM Tris pH8.0, 0.1 mM EDTA overnight and then again into fresh buffer to remove the remaining CsCl. The phage are collected from the cassette and a portion is used to calculate the titre of the phage solution by infecting a dilution of the phage into E. coli TG1 which have been grown to an A₆₀₀ of around 0.6.

The ProcM produced by a recombinant host cell comprising the synthethised gene and expressing the enzyme is used to add thioether bonds to the library. The library is cyclised by adding 50 mM HEPES pH 7.5 to 20 ml and TCEP to a final concentration of 1 mM. Following incubation at room temperature for 30 minutes ⅕^{th} volume of PEG/NaCl solution is added for 5 minutes at room temperature to precipitate the phage. The phages are collected by centrifugation and the supernatant is decanted off. The phages are resuspended in 10 ml of modification buffer 50 mM HEPES pH 7.5, 0-1000 mM NaCl, 1-10 mM MgCl₂, 0.5-10 mM ATP, 0.1 mM TCEP. ProcM is then added at 0.5 μM to 100 μM final concentration and the solution incubated, with gentle mixing, at between 10° C. and 37° C., preferably 25° C. for between 1 and 24 hours. The phage are precipitated as before and resuspended in 10 mM Tris pH 8.0, 0.1 mM EDTA. After titrating the phage the library is diluted to 2e¹² phage per ml and ⅓ volume of ethylene glycol added before storage at −20° C.

An alternative modification procedure uses lysate from Prochlorococcus marinus. The Prochlorosin library is cyclised by adding 50 mM HEPES pH 7.5 to 20 ml and TCEP to a final concentration of 1 mM. Following incubation at room temperature for 30 minutes ⅕^{th} volume of PEG/NaCl solution is added for 5 minutes at room temperature to precipitate the phage. The phages are collected by centrifugation and the supernatant is decanted off. The phage are resuspended at 1e¹² phage ml⁻¹, 1e¹¹ phage ml⁻¹ or 1e¹² phage ml⁻¹ in 50 mM HEPES pH 7.5, 1-10 mM MgCl₂, 0.5-10 mM ATP, 0.1 mM TCEP. Lysate is added in the ratio of 1:1 phage:lysate, 1:2 phage:lysate, up to 1:10 phage:lysate and the solution incubated, with gentle mixing, at between 10° C. and 37° C., preferably 25° C. for between 1 and 24 hours. The phages are precipitated as before and resuspended in 10 mM Tris pH 8.0, 0.1 mM EDTA and are used immediately in a selection or stored in ethylene glycol at −20° C.

Cyclisation conditions are optimised using an ELISA assay. Conditions with a higher signal “are assumed” to have a higher proportion of correctly modified cyclic peptide, because the phage titre does not vary significantly between isolated clones. A comparison is made with non-modified peptide to show that the non-cycled (e.g. linear) peptide is not responsible for the binding.

Between rounds of selection, the library output is modified on a small scale. After overnight growth of the phage infected E. coli TG1 cells in 2TY+tetracycline media, 1 ml of media has 1 μl of 1M TCEP added with mixing for 20 minutes. The cells are removed by centrifugation and the supernatant is added to 200 μl of PEG/NaCl solution at RT for 5 minutes. The phages are harvested by centrifugation and are resuspended in 500 μl of modification buffer from above. ProcM is added and the mixture is at between 10° C. and 37° C., preferably 25° C. for between 1 and 24 hours with gently mixing.

Alternatively, 1 ml of TG1 culture has 50 μl of 1M NaHCO₃ solution containing 1 mM TCEP and 10 μl of magnetic anion exchange resin added for 20 minutes at RT with mixing. The resin is harvested magnetically and washed in 20 mM NaHCO₃, 0.1 mM TCEP. The resin is transferred to modification solution as above. After the incubation, the resin is washed as before and the phages are eluted from the resin in buffer containing 50 mM citrate at pH between 3.5 and 5.0, NaCl between 1 and 2M. The solution is neutralised with 1M Tris pH 9.

Selections are performed using panning on plastic microtitre plates or preferably using biotinylated target in solution. Biotinylated target at a concentration 20 nM to 500 nM is mixed with library in 500 μl of buffer for 1 hr. Magnetic streptavidin beads are added and mixed for 5 minutes. The beads are magnetically captured and washed 3 to 8 times in buffer. The phage are eluted from the beads using 50 mM glycine pH 2.2, or using other methods such protease digestion of the target/peptide complex, ultrasonic elusion, direct infection of E. coli with bead bound phage, disulphide reduction of the linkage between the peptide the phage or within the streptavidin conjugating reagent. Eluted phage are neutralised with 1M Tris pH 8.0 and are used to infect E. coli.

The quality of the library is judged initially by the difference in titre between selections performed with target and those without target at round three and round four of the selection. Where there is a difference in output number, individual colonies are picked and grown for phage production and for sequencing. The phages are modified and screened for binding in the homogeneous phage binding assay, or in ELISA or DELFIA binding assays. The library is of high quality if more than one family of peptide sequence and structure (motifs) is isolated to a particular target.

Selections are also performed with non-modified library. Positive binders identified from these selections are treated under the conditions for cyclisation of the peptide. The binding signal with the cyclised peptide is very low compared to the non-cyclised peptide, showing that there is a high level of modification of the peptide on the phage. In both cases the leader was removed with trypsin, or TEV, or another suitable protease, showing that binding was not due to the leader sequence.

### Example 2: Production of the Fd Bacteriophage Vector Fd-SAN

FD-TET (LGC Standards, Austria) containing E. coli was grown in 2TY media (Melford Biolaboratories Ltd, UK)+12.5 μg/ml tetracycline (Melford Biolaboratories Ltd, UK) (2TY/TET) overnight at 37° C., 250 rpm. 2 ml of the overnight culture was centrifuged at 13000 rpm for 2 minutes. The supernatant was filtered through a 0.2 μm filter (Sartorius, Austria) into a culture of CJ236 E. coli (New England Biolabs, Austria), which had been grown in 5 ml of 2TY medium to an optical density absorption at 600 nm (A600) of 0.6. After 1 hour at 37° C., 250 rpm, the culture was plated onto LB agar (Melford Biolaboratories Ltd, UK) containing 12.5 μg/ml tetracycline. After overnight growth at 37° C., a single colony was picked and grown in 2 ml of 2TY/TET. When the A600 had reached 1.0, 0.1 ml of the culture was added to 30 ml of 2TY/TET containing 0.25 μg/ml uridine (Sigma, Austria). After overnight growth at 37° C., 250 rpm, the cells were removed by centrifugation at 9400 g. The supernatant was collected and 6 ml of 2.5 M NaCl, 20% w/v PEG 8000 (Sigma, Austria) was added at room temperature (RT) for five minutes to precipitate the phage. The precipitated phages were collected by centrifugation at 9400 g for 10 minutes at 4° C. The phage were resuspended in 0.5 ml of PBS (Formedia, UK) and centrifuged at 9400 g for 10 minutes at 4° C. to remove any particulates. The uridine is incorporated by CJ236s into the DNA as uracil and the uracil containing single stranded DNA (dU-ssDNA) from the phage was purified using a column from the QIAprep Spin M13 Kit (Qiagen, UK) according to the manufacturer's instructions. The concentration of the DNA was determined, by measuring the absorbance of light at 260 nm (A260), and then used in a Kunkel mutagenesis (see below) using the following phosphorylated oligo (Integrated DNA Technologies, USA): CTAACAACTTTCAACAGTTTCTGCGGCCGCCCCGTGCACCGCCATGGCCGGCT GGGCCGCATAGAAAGGAACAACTAAAGG (SEQ ID 103) to introduce a Sfil, an ApaLI and a NotI restriction site between the DNA coding for leader sequence of gene 3 protein and domain one of gene 3 protein of the phage, generating a vector called FD-SAN.

To produce the fd-SAN vector a Kunkel mutagenesis approach was performed as follows: 10 μg of the dU-ssDNA, 0.263 μg phosphorylated oligo (1:3 molar ratio), 25 μl TM buffer (0.5 M Tris-HCl pH 7.5 (Sigma, Austria), 0.1 M MgCl₂ (Melford Biolaboratories Ltd, UK) and water to 250 μl final volume), were mixed then split between two PCR tubes. The oligo was annealed to the vector by heating to 90° C. 2 min, 50° C. for 3 min and 20° C. for 5 min in a PCR machine.

The following mix, 10 μl 10 mM ATP (Melford Biolaboratories Ltd, UK), 10 μl 25 mM dNTPs (New England Biolabs, Austria), 15 μl 100 mM DTT (Melford Biolaboratories Ltd, UK), 1 μl T4 DNA ligase (New England Biolabs, Austria), 3 μl T7 polymerase (New England Biolabs, Austria) was divided between the PCR tubes followed by incubation at 20° C. for three hours. The contents of the tubes were pooled and the DNA was purified using a QIAquick PCR Purification Kit column according to the manufacturer's instructions.

TG1 E. coli (Lucigen, Germany) were grown to an A600 of 0.6 in 25 ml of 2TY media. The media was then chilled on ice for 30 minutes. The cells were collected by centrifugation at 2000 g for 10 minutes. The supernatant was carefully decanted off and 50 ml of ice-cold HyClone HyPure WFI quality water (Fisher, Austria) was used to resuspend the cells. The cells were centrifuged and resuspended in water as before twice more. After the final centrifugation, the supernatant was carefully decanted off and the cells were resuspended in 400 μl of the same water. 50 μl of the cell suspension was mixed with 1 μl of the Kunkel mutated DNA in a 1 mm gap electroporation cuvette and electroporated using a BioRad micropulser (Biorad, Austria) on setting EC1. Immediately after the electroporation, 1 ml of SOB media (Melford Biolaboratories Ltd, UK), plus 2% v/v 1 M glucose (ThermoFisher, Austria) was added and the contents transferred to a 14 ml culture tube. After 1 hour at 37° C., 250 rpm the culture was plated onto LB agar/tetracycline plates before overnight growth at 37° C.

After the overnight growth, four colonies were picked into 20 μl of water each. 9 μl of each of these samples was added to four tubes containing 10 μl each of OneTaq Quick-Load PCR mix (New England Biolabs, Austria) and 1 μl of a mix of the primers SeqF GCGATGGTTGTTGTCATTGT (SEQ ID 104) and SeqR ATTCCACAGACAGCCCTCAT (SEQ ID 105) at 10 μM each (Integrated DNA Technologies, USA). The following PCR program was used to amplify the DNA: 3 minutes at 94° C. followed by 35 cycles of 94° C. for 15 seconds, 65° C. for 15 seconds, 68° C. for 15 seconds, followed by 5 minutes at 68° C. The samples were purified using QIAquick PCR Purification Kit column according to the manufactures instructions and were sequenced by MicroSynth (Austria) using the SeqF primer. Sequencing confirmed that FD-SAN had been correctly made.

### Example 3: Production of Bacteriophage Vector Fd ProcLeader

ProcA 4.3 leader sequence (Accession no WP 011130304) with a 5′ Sfil and a 3′ NotI cloning site for in-frame cloning into gene 3 in FD-SAN was ordered from Genewiz (Genewiz, USA). The vector containing the ProcA 4.3 leader sequence and FD-SAN was prepared as double stranded DNA using a HiSpeed Plasmid Midi Kit (Qiagen, Austria). 1 μg of each vector was digested with 1 μl of Sfil and NotI (Fermentas, Austria) according the manufacturer's instructions in a 50 μl volume. FD-SAN was purified using a QIAprep PCR purification kit column and the ProcA 4.3 leader fragment was isolated from an agarose gel after electrophoresis using a QIAprep Gel purification kit column (Qiagen, Austria). The fragments were ligated together using Quick Ligase kit (New England Biolabs, Austria) according to the manufacturer's instructions and were transformed into TG1 cells by electroporation as above. Colonies that resulted from the overnight growth of the transformed cells on LB agar+tetracycline plates were sequenced to identify clones where the insert was correctly in frame in the vector. The new vector was called FD-ProcA4.3.

A phosphorylated, PAGE purified oligo (Sigma-Genosys, UK) CTGCGGCCGCGCCTGCGCASNNSNNSN NGGWCGGAGWSN NSNNSNNGCAGGC CGCGCCGCCCGCC (SEQ ID 106) was made to mutate FD-ProcA4.3 into a vector that would display downstream of the ProcA4.3 leader a novel prochlorosin library with the sequence AACXXXDha/DhbPDha/DhbXXXC (SEQ ID 107), after cleavage of the gene 3 leader sequence on the phage and modification by ProcM enzyme, called SynLib1. X is any amino acid, Dha is dehydrated serine and Dhb is dehydrated threonine, which would be made by the ProcM.

dU-ssDNA of FD-ProcA4.3 was prepared as described above for the preparation of FD-TET, using 600 ml of culture (20× the scale). All steps for the preparation of the library were performed as above on a 2× scale except for the electroporation being performed in 2 mm cuvettes using 370 μl of cells and 30 μl of DNA on setting EC2 and 2 ml of SOC being used to collect all of the cells from the cuvette. After 10 electroporations, the volume of the cells was made up to 50 ml with SOC and the cells were allowed to recover at 37° C., 250 rpm for an hour. The number of transformants in the library was determined by making serial dilution of the cells, which were then plated on LB agar/tetracycline plates. The remainder of the culture was made up to 2 l with 2TY+tetracycline media (in four 2 l flasks) and grown overnight at 37° C., 250 rpm. From the titre plate the library was determined to contain 1.5e10 members.

The 2 l of culture from the overnight growth was chilled on ice for 30 minutes, centrifuged at 9400 g for 10 minutes at 4° C. and the supernatant was collected. 400 ml of 2.5 M NaCl, 20% w/v PEG 8,000 was added on ice for 30 minutes to precipitate the phage before centrifugation at 9400 g for 10 minutes at 4° C. The supernatant was decanted off and the precipitated phage were resuspended in 40 ml of PBS. This was centrifuged at 9400 g for 10 minutes at 4° C. to remove any remaining particulate matter. The supernatant was transferred to a clean tube and 8 ml of 2.5 M NaCl, 20% w/v PEG 8000 was added to precipitate the phage. After 5 minutes at RT the precipitated phage were collected by centrifugation at 9400 g for 5 minutes at 4° C. The supernatant was decanted off and the phage were resuspended in 20 ml of TE buffer (10 mM Tris pH 8.0 (Sigma, Austria), 0.1 mM EDTA (Melford Biolaboratories, UK)) followed by the addition of 20 ml of ethylene glycol (Melford Biolaboratories, UK) and storage at −20° C. The phage titre was determined by infecting a dilution of phage into TG1 cells that had been grown to A600 of 0.6, incubated at 37° C., 250 rpm for an hour and then plated on LB agar+tetracycline plates in a range of dilutions.

### Example 4: Heterologous Production of Class II Lanthipeptide Synthetase ProcM

The amino acid sequence of class II lanthipeptide synthetase of ProcM (Accno CAE20425) was used as a template to produce a synthethic gene coding for ProcM (Genewhiz, USA). The synthetic gene coding for ProcM was cloned into expression plasmid pET28b (Novagen, USA) using restriction endonucleases NdeI and EcoRV (NEB, Austria) following the recommendations of the supplier. DNA sequencing (Microsynth, Austria) was used to confirm correct recombinant DNA plasmids named construct ProcM-pET28b.

10 ng of plasmid ProcM-pET28b was used to genetically transform electro-competent Escherichia coli BL21 (DE3) (Novagen, USA) using the electroporator Micropulser (BioRad, Austria) with standard setting for E. coli and standard 2 mm width cuvettes (Sigma Aldrich, Austria). Resulting single clones were selected on Kanamycin (Melford Biolaboratories UK) containing LB (Sigma Aldrich) petri dishes (GreinerBioOne, Austria) and one single clone was used to inoculate 11 of Kanamycin contaning LB broth. Cultures were incubated in 21 Erlenmayer flasks (Schott, Germany) each containing 500 ml suspension at 37° C. with constant shaking at 220 rpm on a shaker (Innova, Switzerland). Optical density was monitored by spectrophotometrical measurements at 600 nm with a U-2800 spectrophotometer (Hitachi, Austria). After reaching an optical density of 0.6 the suspension was cooled on ice for 5 minutes and supplemented with 0.1 mM IPTG (Isopropyl-β-D-thiogalactopyranosid) (Melford Biolaboratories, UK) and incubated further at 16° C. with shaking at 220 rpm for additional 16 h.

After 16 h cells were sedimented by centrifugation (Sorvall Instruments, USA) for 10 min at 6000 g. The resulting cell paste was frozen at −20° C. in a standard freezer (Liebherr, Austria). Frozen cell paste was weighed and resuspended in 10× (weight per volume) of Lysis buffer. Lysis buffer contained 50 mM Tris (Isopropyl-β-D-thiogalactopyranosid) (Sigma Aldrich, Austria), 300 mM NaCl (Sigma Aldrich, Austria), 5 mM Imidazole (Amresco, USA) pH 8. Cells were disrupted in a M-110P cell disruptor (Microfluidics, USA) until suspension became translucent (one to two cycles). Cell debris and the unsoluble protein fraction were separeted by centrifugation (Sorvall Instruments, USA) for 30 min at 4° C. with 15.000 g. The supernatant was then transferred into a new centrifugation tube (Thermo Scientific, Austria) and centrifuged again under identical conditions. After second centrifugation the supernatant was mixed with 500 μl Nickel-Agarose resin (Qiagen, Austria) that was previously washed with 2.5 ml (5 vol) of 50 mM Tris pH7.5 (Sigma Aldrich). The resin-supernatant mixture was incubated in a over-head shaker (Stuart Instruments, Austria) at 4° C. for 2 h at 30 rpm. After 2 h the suspension was loaded on a polyprep column (Biorad, Austria) and allowed to drain by gravity force. The resin was washed with 5 ml lysis buffer with 5 mM beta-mercaptoethanol (BME) (Sigma Aldrich, Austria), followed by 3×1 ml wash buffer (50 mM TRIS, 300 mM NaCl, 25 mM imidazole, 5 mM BME pH 8). The protein was eluted with 3×1 ml of elution buffer (50 mM TRIS, 300 mM NaCl, 250 mM Imidazole, 5 mM BME, pH 8).

The elution fractions were combined and desalted using a desalting column (Econopac, Biorad, Aistria) equilibrated with storage buffer (25 mM TRIS, 500 mM NaCl, 10% (w/v) glycerol (Sigma Aldrich, Austria). The protein was aliquoted into 100 μl portions in 0.2 ml tubes (Starlab, Germany) and flash frozen in liquid nitrogen and stored at −80° C. until use.

An aliquot of the protein was checked by SDS-PAGE gel electrophoresis to confirm the purity of the produced class II lanthipeptide synthease ProcM.

### Example 5: Preparation of Human Plasma Kallikrein as Target for Phage Selection

Human Plasma Kallikrein (Molecular Innovations, Inc, USA) at 1.15 mg/ml (13 μM) solution in 4 mM acetate, 150 mM NaCl, pH 5.3 was biotinylated using NHS-PEG4-Biotin from the EZ-link micro-PEO4-biotinylation kit (Pierce Biotechnology, USA). 680 μl of water was added to the contents of the pre-weighed vial of biotinylation reagent to make a 5 mM solution. 1 μl of this solution was added to 100 μl of the kallikrein solution. This was incubated at RT for 1 hour prior to the separation of the biotinylated protein from the unreacted biotin, using a 2 ml Spin Desalting column (Pierce Biotechnology, USA). The volume of the kallikrein was made up to 350 μl with PBS (Formedium Ltd, UK) and was loaded onto the spin column which had been equilibrated with PBS (as per the manufacturer's instructions). When this solution had been absorbed into the column matrix, a further 50 μl of PBS was added on top and the column was spun in a centrifuge at 1000 g for 2 min. 395 μl of solution was collected, making the concentration of the biotinylated kallikrein about 3.25 μM.

### Example 6: Modified Phage Selection Procedure

150 μl of stock SynLib1 library (2.5e11 phage per μl) was added to 1850 μl of TE buffer, 2 μl 1 M TCEP (Melford Biolaboratories Ltd, UK) and incubated at RT for 20 minutes to reduce the peptides on the phage. 400 μl of 2.5 M NaCl, 20% w/v PEG 8000 was added, incubated at RT for five minutes and the precipitated phage were collected by centrifugation at 15000 g for five minutes. The supernatant was discarded and the phage were resuspended in 150 μl of 25 mM Tris pH 7.5 (Sigma Aldrich, Austria), 1 μM TCEP. 10 μl of phage-containing solution, 40 μl 10 mM ATP (Melford Biolaboratories Ltd, UK), 1 μl 2 M MgCl₂ (Melford Biolaboratories Ltd, UK), 1 μl 200 μM TCEP and 150 μl of WT ProcM were mixed and incubated at 25° C. overnight to allow the enzyme to modify the peptide displayed on the phage.

Selections are performed by removing streptavidin-binding clones by pre-exposing the library to streptavidin resin, which is then discarded, mixing the library with decreasing concentrations of biotinylated target for an hour at each round of selection, capture of the biotinylated target and thereby any phage binding the target with magnetic streptavidin resin followed by extensive washing of the resin. Finally, the washed resin has the phage eluted from the target by exposure to a low pH solution, neutralisation of the solution and infection of E. coli by the eluted phage.

Round 1 (R1) of the selection was performed by adding 50 μl of LodeStars streptavidin resin (Agilent Technologies, USA), from which the liquid had been removed by magnetic capture, to the modified phage solution for 30 minutes to capture the peptide sequence that bind to streptavidin (deselection). The resin was captured magnetically and the phage-containing supernatant was collected and made up to 500 μl with 25 mM Tris pH 7.5, 0.05% Tween 20 (Melford Biolaboratories Ltd, UK). Biotinylated kallikrein was added to 100 nM, and mixed at RT for 1 hour prior to processing on a KingFisher mL (Thermo Fisher, Austria) magnetic particle washer. The processing involved the KingFisher transferring 25 μl of streptavidin resin to the phage solution and mixing for five minutes. The resin/phage were then collected and washed in 8×1 ml of PBS+0.05% Tween 20 (PBST). The resin was then mixed in 50 μl of 50 mM glycine pH 2.2 (Melford Biolaboratories Ltd, UK) for five minutes to elute the phage and the resin was removed. The phage-containing supernatant was neutralised with 10 μl of 1 M Tris pH 8.0 and added to 1 ml of TG1 cells at A600 of 0.6 followed by incubation at 37° C., 250 rpm for 1 hour. The phage titre was determination by adding 4 μl of culture to 96 μl of water, which was used to make five, 10-fold dilutions; 10 μl from each of these was spotted onto an LB+tetracycline agar plate. The remainder of the culture was made up to 11 ml with 2TY plus tetracycline and incubated at 37° C., 250 rpm overnight.

10 ml of overnight culture was reduced with 10 μl of 1 M TCEP for 20 min at RT followed by removal of the cells by centrifugation at 15000 g for five minutes. The supernatant was transferred into a new tube containing 2.5 ml of PEG/NaCl and mixed, incubated at RT for five minutes and centrifuged at 15000 g for five minutes to collect the precipitated phage. The supernatant was carefully decanted off and the phage were resuspended in 500 μl of 25 mM Tris pH 7.5, 1 μM TCEP. The enzymatic modification of the phage was set up as before, except that 20 μl of phage solution was used. For the round 2 (R2) input, the phage and enzyme mix were incubated at 25° C. overnight and at round 3 (R3) the incubation was for 4 hours at 25° C.

R2 selection was performed as for R1 at 50 nM kallikrein. For R3 the modified phage was made up to 1000 μl with 25 mM Tris pH 7.5, 0.05% Tween 20 after the deselection and split into two lots of 500 μl, which were used for selections using either 6.25 or 0 nM kallikrein.

The number of phages which are eluted at the end of each round of selection is known as the output titre. The output titre at each round of selection was 2.1e6 R1, 1.6e5 R2 and 5.3e5 R3 plus kallikrein and 5.3e4 R3 minus kallikrein. The increase in titre between R2 and R3 combined with the 10-fold higher titre 6.25 nM kallikrein over the R3 0 nM kallikrein indicate that the selections have successfully isolated peptides that bind to kallikrein.

24 clones from the R3 6.25 nM kallikrein output were sequenced as above giving the following sequences for the displayed peptide.

| | | | | | | | | | | | | | | SEQ |
|---|---|---|---|---|---|---|---|---|---|---|---|---|---|---|
| clone | | | | | | | | | | | | | | ID |
| # | | | | | | | | | | | | | | NO |
| 24 | A | A | C | K | G | P | S | P | T | P | N | N | C | 108 |
| 2 | A | A | C | K | G | P | S | P | T | P | N | N | C | 108 |
| 21 | A | A | C | K | G | P | S | P | T | P | N | N | C | 108 |
| 15 | A | A | C | S | R | S | S | P | S | R | T | Y | C | 109 |
| 23 | A | A | C | S | R | S | S | P | S | R | T | Y | C | 109 |
| 6 | A | A | C | T | N | S | S | P | T | Q | V | T | C | 110 |
| 17 | A | A | C | N | D | A | S | P | S | P | N | M | C | 111 |
| 1 | A | A | C | K | L | R | T | P | S | N | Q | Q | C | 112 |
| 16 | A | A | C | H | I | G | S | P | S | I | N | Q | C | 113 |
| 12 | A | A | C | G | P | T | T | P | T | S | P | P | C | 114 |
| 18 | A | A | C | P | G | P | T | P | T | N | M | P | C | 115 |
| 13 | A | A | C | M | Y | L | T | P | T | R | S | L | C | 116 |
| 20 | A | A | C | T | I | F | T | P | S | P | F | F | C | 117 |
| 8 | A | A | C | T | A | P | S | P | S | V | T | Q | C | 118 |
| 5 | A | A | C | L | K | P | T | P | T | Q | S | T | C | 119 |
| 10 | A | A | C | S | K | G | S | P | S | P | S | H | C | 120 |
| 14 | A | A | C | T | S | H | T | P | T | H | K | N | C | 121 |
| 9 | A | A | C | V | Q | T | T | P | T | L | I | G | C | 122 |
| 3 | A | A | C | P | V | S | T | P | T | L | T | S | C | 123 |
| 22 | A | A | C | S | F | Q | S | P | S | L | T | H | C | 124 |
| 7 | A | A | C | S | N | T | T | P | T | P | D | Y | C | 125 |
| 4 | A | A | C | Q | S | N | T | P | T | R | T | P | C | 126 |
| 11 | A | A | C | L | S | T | S | P | S | K | I | P | C | 127 |
| 19 | A | A | C | T | M | A | T | P | S | K | N | G | C | 128 |

As can be seen from the sequencing there was a strong selection pressure for the sequence AACKGPSPTPNNC (SEQ ID 108), which was found in clones 24, 2 and 21. This clone represented 12.5% of the clones that were sequenced. Clones 15 and 23 are identical, AACSRSSPSRTYC (SEQ ID 109) representing another clone to be seen multiple times in the selection.

No clones containing the well-known HPQ streptavidin-binding motif were seen, suggesting that the deselection using the streptavidin beads before the selection was successful.

The multiple times that two clones were seen, combined with the enrichment between rounds 2 and 3, the 10-fold difference in titre at R3 between the 6.25 nM containing selection and the 0 nM containing selection show that this is the first successful phage selection using the prochlorosin system.

### Example 7: Heterologous Production of Class II Lanthipeptide Synthetase ProcM Fused to the N-Terminal Peptide of Prochlorosin ProcA4.3 (Enzyme-Leader-Fusions, ELFs)

Heterologous production of the class II lanthipeptide synthetase ProcM fused to the N-terminal peptide of Prochlorosin ProcA4.3 (Enzyme-leader-fusions, ELFs) were made as follows. ELFs were made in a three step procedure where, first the enzyme and leader sequence were cloned, second, variants of each were made with N- and C-terminal extensions of 5 (SEQ ID 167), 10 (SEQ ID 168, SEQ ID 170) or 15 (SEQ ID 169, SEQ ID 171) Glycine/Serine, Alanine/Serine, Threonine/Serine amino acid pairs, third, overlapping PCR was used to join these together to make the constructs leader-G/S(5)-ProcM, leader-G/S(10)-ProcM, leader-G/S(5)-ProcM, and ProcM-G/S(5)-leader, ProcM-G/S(10)-leader, ProcM-G/S(15)-leaderas follows.

The amino acid sequence of class II lanthipeptide synthetase of ProcM (Accession no CAE20425) was used as a template to produce a synthetic gene coding for ProcM (Genewiz, USA). The synthetic gene coding for ProcM was cloned into plasmid pUC57 and named ProcM-pUC57. Two DNA oligonucleotides phosphorylated at their 5′ ends and complementary to each other coding for the N-terminal leader portion of ProcA4.3 (Accession no WP 011130304) were synthesized (Mircosynth, Austria) and hybridized with each other following standard procedures (Sambrock et al). The resulting double stranded DNA was cloned into pJET1.2 (ThermoFisher, Austria) and DNA sequencing was used to confirm correct DNA sequences. The resulting construct was named LeaderA4.3-pJET and used as DNA template for subsequent PCR reactions. Six different PCR reactions each using 10 ng plasmid DNA LeaderA4.3-pJET were made with differing oligonucleotide primer combinations. Three PCR reactions containing the forward primer (NdeINelfLeader+) which has a 5′NdeI restriction site and complementary to the 5′ end of the DNA of plasmid LeaderA4.3-pJET and specific reverse primers, each being complementary to the 3′ end of LeaderA4.3 DNA sequence and differing by individual 5′ extensions coding for an extra Glycine/Serine, Alanine/Serine, Threonine/Serine (NelfLeaderGS5−) or 10 Glycine/Serine, Alanine/Serine, Threonine/Serine (NelfLeaderGS10−) or 15 Glycine/Serine, Alanine/Serine, Threonine/Serine (NelfLeaderGS15−) amino acid pairs were performed using Phusion DNA polymerase according to the manufacturer's instructions (ThermoFisher, Austria). Resulting PCR products were isolated using agarose gel electrophoresis following standard procedures (Sambrock et al), purified using QiaQuick gel extraction kit (Qiagen, Austria) and stored at −20° C. The second set of three PCR reactions containing the reverse primer (EcoRICelfLeader−) which has a 5′EcoRI restriction site and complementary to the 5′ end of DNA LeaderA4.3-pJET and specific forward primers, each being complementary to the 3′ end of LeaderA4.3 DNA sequence and differing by individual 5″extensions coding for an extra 5 Glycine/Serine, Alanine/Serine, Threonine/Serine(CelfLeaderGS5+) or 10 Glycine/Serine, Alanine/Serine, Threonine/Serine (CelfLeaderGS10+) or 15 Glycine/Serine, Alanine/Serine, Threonine/Serine (CelfLeaderGS15+) amino acid pairs were performed using Phusion DNA polymerase according to the manufacturer's instructions (ThermoFisher, Austria). Resulting PCR products were isolated using agarose gelchromatography (Lonza, USA) and stained with PeqGreen dye (Peglab, Germany) and visualized under UV light (UVP, Germany). Bands representing the correct size were cut out from the gel, purified using QiaQuick gel extraction kit (Qiagen, Austria) and stored at −20° C.

60 ng of plasmid DNA ProcM-pUC57 was used for six PCR amplifications using different primer combinations. A subset of three PCR reactions contained the same reverse primer (EcoRINelfProcM−) complementary to the 3′ end of synthetic gene ProcM from plasmid ProcM-pUC57. To each reaction a specific forward primer was added with each being complementary to the 5′ end of ProcM DNA and differing by individual 5″extensions coding for an 5 Glycine/Serine, Alanine/Serine, Threonine/Serine (NelfProcMGS5+), or 10 Glycine/Serine, Alanine/Serine, Threonine/Serine (NelfProcMGS10+) or 15 Glycine/Serine, Alanine/Serine, Threonine/Serine (NelfProcMGS15+) amino acid pairs. PCR reactions were performed using Phusion DNA polymerase according to the manufacturer's instructions (ThermoFisher, Austria). Resulting PCR products were isolated using agarose gelchromatography (Lonza, USA) and stained with PeqGreen dye (Peqlab, Germany) and visualized under UV light (UVP, Germany). Bands representing the correct size were cut out from the gel, purified using QiaQuick gel extraction kit (Qiagen, Austria) and stored at −20° C. The second subset of three PCRs were containing the same forward primer (NdeICelfProcM+) complementary to the 5′ of the synthetic gene ProcM from plasmid ProcM-pUC57. To each reaction a specific reverse primer was added with each being complementary to the 5′ end of ProcM DNA and differing by individual 5″extensions coding for an extra 5 Glycine/Serine, Alanine/Serine, Threonine/Serine (CelfProcMGS5−), or 10 Glycine/Serine, Alanine/Serine, Threonine/Serine (CelfProcMGS10−) or 15 Gly-cine/Serine, Alanine/Serine, Threonine/Serine (CelfProcMGS15−) amino acid pairs. PCR reactions were performed using Phusion DNA polymerase according to the guidelines of the supplier (ThermoFisher, Austria). Resulting PCR products were isolated using agarose gelchromatography (Lonza, USA) and stained with PeqGreen dye (Peqlab, Germany) and visualized under UV light (UVP, Germany). Bands representing the correct size were cut out from the gel, purified using QiaQuick gel extraction kit (Qiagen, Austria) and stored at −20° C.

The amplicons from above were used as DNA templates for overlapping PCR reactions. PCR products NelfLeaderGS5 was used together with NdeINelfProcMGS5, NelfLeaderGS10 together with NdeINelfProcMGS10 and NelfLeaderGS15 in combination with NdeINelfProcMGS15. For each DNA template combination the outside primers of NdeINelfLeader+ and EcoRINelfProcM− were used and DNA polymerase Q5 (New England Biolabs, Austria) following the manufacturer's instructions. Resulting PCR products were isolated using agarose gelchromatography (Lonza, USA) and stained with PeqGreen dye (Peqlab, Germany) and visualized under UV light (UVP, Germany). Bands representing the correct size were cut out from the gel, purified using QiaQuick gel extraction kit (Qiagen, Austria) and cloned into pJET1.2. Resulting recombinant plasmids were used for DNA sequencing to confirm correct DNA sequences. Resulting plasmids were named Nelf5ProcM, Nelf10ProcM, Nelf15ProcM for N-terminal leaderA4.3 ProcM fusions.

The remaining amplicons from above were used as DNA templates for overlapping PCR reactions. PCR products CelfLeaderGS5 was used together with EcoRICelfProcMGS5, CelfLeaderGS10 and EcoRICelfProcMGS10 and CelfLeaderGS15 in combination with EcoRICelfProcMGS15. For each DNA template combination the outside primers of EcoRICelfLeader− and NdeICelfProcM+ were used and DNA polymerase Q5 (New England Biolabs, Austria) following the manufacturer's instructions. Resulting PCR products were isolated using agarose gelchromatography (Lanza, USA) and stained with PeqGreen dye (Peqlab, Germany) and visualized under UV light (UVP, Germany). Bands representing the correct size were cut out from the gel, purified using QiaQuick gel extraction kit (Qiagen, Austria) and cloned into pJET1.2. Resulting recombinant plasmids were used for DNA sequencing to confirm correct DNA sequences. Resulting plasmids were named Celf5ProcM, Celf10ProcM, Celf15ProcM for C-terminal leaderA4.3 ProcM fusions.

3 μg of each of the above produced plasmid DNAs were digested with NdeI and EcoRI and resulting fragments were separated by agarose gelchromatography. DNA bands of the expected sizes were isolated using QiaQuick gel extraction kit and cloned into expression plasmid pET28b (Novagen, USA), which was digested with NdeI and EcoRI. DNA sequencing (Microsynth, Austria) was used to confirm correct recombinant DNA plasmids named Nelf5ProcM-pET28b, Nelf10ProcM-pET28b, Nelf15ProcM-pET28b and Celf5ProcM-pET28b, Celf10ProcM-pET28b and Celf15ProcM-pET28b.

10 ng of each of the above described plasmids were used to genetically transform electro-competent Escherichia coli BL21 (DE3) (Novagen, USA) using the electroporator Micropulser (BioRad, Austria) with standard setting for E. coli and standard 2 mm width cuvettes (Sigma Aldrich, Austria). Resulting single clones were selected on Kanamycin (Melford Biolaboratories Ltd, UK) containing LB (Sigma Aldrich) petri dishes (GreinerBioOne, Austria) and one single clone was used to inoculate 11 of Kanamycin containing LB broth. Cultures were incubated in 21 Erlenmayer flasks (Schott, Germany) each containing 500 ml suspension at 37° C. with constant shaking at 220 rpm on a shaker (Innova, Switzerland). Optical density was monitored by spectrophotometrical measurements at 600 nm with a U-2800 spectrophotometer (Hitachi, Austria). After reaching an optical density of 0.6 the suspension was cooled on ice for 5 minutes and supplemented with 0.1 mM IPTG (Isopropyl-β-D-thiogalactopyranosid) (Melford Biolaboratories, UK) and incubated further at 16° C. with shaking at 220 rpm for additional 16 h.

After 16 h cells were sedimented by centrifugation (Sorvall Instruments, USA) for 10 min at 6000 g. The resulting cell paste was frozen at −20° C. Frozen cell paste was weighed and resuspended in 10× (weight per volume) of Lysis buffer 50 mM Tris (Sigma Aldrich, Austria), 300 mM NaCl (Sigma Aldrich, Austria), 5 mM Imidazole (Amresco, USA) pH 8. Cells were disrupted in a M-110P cell disruptor (Microfluidics, USA) until suspension became translucent (one to two cycles). Cell debris and the insoluble protein fraction were separated by centrifugation (Sorvall Instruments, USA) for 30 min at 4° C. with 15000 g. The supernatant was then transferred into a new centrifugation tube (Thermo Scientific, Austria) and centrifuged again under identical conditions. After second centrifugation the supernatant was mixed with 500 μl Nickel-Agarose resin (Qiagen, Austria) that was previously washed with 2.5 ml (5 vol) of 50 mM Tris pH7.5 (Sigma Aldrich). The resin-supernatant mixture was incubated in a tube rotator (Stuart Instruments, Austria) at 4° C. for 2 h at 30 rpm. After 2 h the suspension was loaded on a polyprep column (Biorad, Austria) and allowed to drain by gravity force. The resin was washed with 5 ml lysis buffer containing 5 mM beta-mercaptoethanol (BME) (Sigma Aldrich, Austria), followed by 3×1 ml wash buffer (50 mM Tris, 300 mM NaCl, 25 mM imidazole, 5 mM BME pH 8). The protein was eluted with 3×1 ml of elution buffer (50 mM Tris, 300 mM NaCl, 250 mM Imidazole, 5 mM BME, pH 8).

The elution fractions were combined and desalted using a desalting column (Econopac, Biorad, Austria) equilibrated with storage buffer (25 mM Tris, 500 mM NaCl, 10% (w/v) glycerol (Sigma Aldrich, Austria). The protein was aliquoted into 100 μl portions in 0.2 ml tubes (Starlab, Germany) and flash frozen in liquid nitrogen and stored at −80° C. until use.

An aliquot of each of the proteins was checked by SDS-PAGE gel electrophoresis to confirm the purity of the produced class II lanthipeptide synthease ProcM leader fusions.

### Example 8: Preparation of Human Plasma Kallikrein as Target for Phage Selection

Human Plasma Kallikrein (Molecular Innovations, Inc, USA) at 1.15 mg/ml (13 μM) solution in 4 mM acetate, 150 mM NaCl, pH 5.3 was biotinylated using NHS-PEG4-Biotin from the EZ-link micro-PEO4-biotinylation kit (Pierce Biotechnology, USA). 680 μl of water was added to the contents of the pre-weighed vial of biotinylation reagent to make a 5 mM solution. 1 μl of this solution was added to 100 μl of the kallikrein solution. This was incubated at RT for 1 hour prior to the separation of the biotinylated protein from the unreacted biotin, using a 2 ml Spin Desalting column (Pierce Biotechnology, USA). The volume of the kallikrein was made up to 350 μl with PBS (Formedium Ltd, UK) and was loaded onto the spin column which had been equilibrated with PBS (as per the manufacturer's instructions). When this solution had been absorbed into the column matrix, a further 50 μl of PBS was added on top and the column was spun in a centrifuge at 1000 g for 2 min. 395 μl of solution was collected, making the concentration of the biotinylated kallikrein about 3.25 μM.

### Example 9: Modified Phage Selection Procedure

150 μl of stock leader-free SynLib1 library (4.5e11 phage per μl) was added to 1850 μl of TE buffer, 2 μl 1 M TCEP (Melford Biolaboratories Ltd, UK) and incubated at RT for 20 minutes to reduce the peptides on the phage. 400 μl of 2.5 M NaCl, 20% w/v PEG 8000 was added, incubated at RT for five minutes and the precipitated phages were collected by centrifugation at 15000 g for five minutes. The supernatant was discarded and the phages were resuspended in 150 μl of 25 mM Tris pH 7.5 (Sigma Aldrich, Austria), 1 μM TCEP. 10 μl of phage-containing solution, 40 μl 10 mM ATP (Melford Biolaboratories Ltd, UK), 1 μl 2 M MgCl₂ (Melford Biolaboratories Ltd, UK), 1 μl 200 μM TCEP and 150 μl of either Celf5ProcM, Celf10ProcM and Celf15ProcM were mixed and incubated at 25° C. overnight to allow the enzyme to modify the peptide displayed on the phage.

Selections are performed by removing streptavidin-binding clones by pre-exposing the library to streptavidin resin, which is then discarded, mixing the library with decreasing concentrations of biotinylated target for an hour at each round of selection, capture of the biotinylated target and thereby any phage binding the target with magnetic streptavidin resin followed by extensive washing of the resin. Finally, the washed resin has the phage eluted from the target by exposure to a low pH solution, neutralisation of the solution and infection of E. coli by the eluted phage.

Round 1 (R1) of the selection was performed by adding 50 μl of LodeStars streptavidin resin (Agilent Technologies, USA), from which the liquid had been removed by magnetic capture, to the modified phage solution for 30 minutes to capture the peptide sequence that bind to streptavidin (deselection). The resin was captured magnetically and the phage-containing supernatant was collected and made up to 500 μl with 25 mM Tris pH 7.5, 0.05% Tween 20 (Melford Biolaboratories Ltd, UK). Biotinylated kallikrein was added to 100 nM, and mixed at RT for 1 hour prior to processing on a KingFisher mL (Thermo Fisher, Austria) magnetic particle washer. The processing involved the KingFisher transferring 25 μl of streptavidin resin to the phage solution and mixing for five minutes. The resin/phage were then collected and washed in 8×1 ml of PBS+0.05% Tween 20 (PBST). The resin was then mixed in 50 μl of 50 mM glycine pH 2.2 (Melford Biolaboratories Ltd, UK) for five minutes to elute the phage and the resin was removed. The phage-containing supernatant was neutralised with 10 μl of 1 M Tris pH 8.0 and added to 1 ml of TG1 cells at A600 of 0.6 followed by incubation at 37° C., 250 rpm for 1 hour. The phage titre was determination by adding 4 μl of culture to 96 μl of water, which was used to make five, 10-fold dilutions; 10 μl from each of these was spotted onto an LB+tetracycline agar plate. The remainder of the culture was made up to 11 ml with 2TY plus tetracycline and incubated at 37° C., 250 rpm overnight.

10 ml of overnight culture was reduced with 10 μl of 1 M TCEP for 20 min at RT followed by removal of the cells by centrifugation at 15000 g for five minutes. The supernatant was transferred into a new tube containing 2.5 ml of PEG/NaCl and mixed, incubated at RT for five minutes and centrifuged at 15000 g for five minutes to collect the precipitated phage. The supernatant was carefully decanted off and the phage were resuspended in 500 μl of 25 mM Tris pH 7.5, 1 μM TCEP. The enzymatic modification of the phage was set up as before, except that 20 μl of phage solution was used. For both the round 2 (R2) and round 3 (R3) input, the phage and enzyme mix were incubated at 25° C. overnight.

R2 selection was performed as for R1 at 33 nM kallikrein. For R3 the modified phage was made up to 1000 μl with 25 mM Tris pH 7.5, 0.05% Tween 20 after the deselection and split into two lots of 500 μl, which were used for selections using either 9.75 or 0 nM kallikrein.

The number of phages which are eluted at the end of each round of selection is known as the output titre. The output titre at each round of selection were for CelfProcM5, CelfProcM10 and CelfProcM15 respectively at R1, 6.4e5, 4.2e6, 2.7e6, at R2, 5.3e5, 2.9e5, 2.7e5, at R3 using 9.75 nM kallikrein 5.0e5, 3.4e5 and 9.5e5 and with 0 nM kallikrein 5.0e4, 5.3e4 and 2.7e4. Although the output titres generally dropped a little between R1 and R2, and remained similar between R2 and R3, there is a significant difference in titre using 9.75 nM kallikrein and using 0 nM kallikrein, for CelfProcM5, 10 fold and for CelfProcM15, 36 fold. With CelfProcM10 there was a 6.5 fold difference the significance of which will be determined by the sequences of the clones.

20 clones from each 9.75 nM R3 selection were sequenced.

The clones from the CelfProcM5 selection which contained a peptide sequence from the mutagenesis used to make the library had the following sequences.

| Clone # | | | | | | | | | | | | | | SEQ ID |
|---|---|---|---|---|---|---|---|---|---|---|---|---|---|---|
| Clone 9 | A | A | C | Q | P | N | S | P | S | K | K | I | C | 129 |
| Clone 18 | A | A | C | M | P | W | T | P | S | L | F | R | C | 130 |
| Clone 17 | A | A | C | T | F | L | S | P | S | T | P | I | C | 131 |
| Clone 15 | A | A | C | T | S | N | T | P | T | T | R | T | C | 132 |
| Clone 20 | A | A | C | T | I | G | S | P | S | L | R | H | C | 133 |
| Clone 4 | A | A | C | L | F | R | T | P | T | P | F | S | C | 134 |

From the CelfProcM10 selections the following sequences were observed.

| Clone # | | | | | | | | | | | | | | SEQ ID |
|---|---|---|---|---|---|---|---|---|---|---|---|---|---|---|
| Clone 19 | A | A | C | K | D | M | T | P | S | P | K | T | C | 135 |
| Clone 12 | A | A | C | K | L | G | T | P | T | D | K | T | C | 136 |
| Clone 14 | A | A | C | M | D | T | S | P | T | P | P | S | C | 137 |
| Clone 6 | A | A | C | A | F | W | S | P | S | I | M | S | C | 138 |
| Clone 18 | A | A | C | P | P | V | S | P | S | N | D | R | C | 139 |

From the CelfProcM15 selections the following sequences were observed.

| Clone # | | | | | | | | | | | | | | SEQ ID |
|---|---|---|---|---|---|---|---|---|---|---|---|---|---|---|
| Clone 2 | A | A | C | T | M | G | T | P | T | Q | S | G | C | 140 |
| Clone 12 | A | A | C | T | N | L | S | P | T | Q | D | Q | C | 141 |
| Clone 13 | A | A | C | H | K | S | S | P | T | Q | V | Y | C | 142 |
| Clone 10 | A | A | C | F | I | R | T | P | S | F | Q | R | C | 143 |
| Clone 1 | A | A | C | P | K | P | T | P | S | A | L | K | C | 144 |
| Clone 20 | A | A | C | L | F | R | S | P | T | P | F | S | C | 145 |
| Clone 15 | A | A | C | H | T | P | T | P | T | T | L | S | C | 146 |
| Clone 11 | A | A | C | S | S | R | T | P | T | T | T | E | C | 147 |
| Clone 17 | A | A | C | N | S | S | T | P | T | P | D | T | C | 148 |
| Clone 7 | A | A | C | N | Y | W | S | P | T | Y | E | D | C | 149 |
| Clone 5 | A | A | C | K | G | A | S | P | S | L | P | K | C | 150 |

Although there are no clones that are seen multiple times, the difference in output titre between the R3 9.75 nM and the R3 0 nM selections suggests that some of these clones may be kallikrein binding peptides.

### Example 10: Heterologous Production of the ATP-Grasp Ribosomal Peptide Maturase MvdD Fused to the N-Terminal Peptide (Leaderpeptide) of the Microviridin Precursorpeptide MvdE (Enzyme-Leader-Fusions, ELFs)

Heterologous production of the ATP-grasp ribosomal peptide maturase MvdD (SEQ ID 75) fused to the N-terminal peptide (Leaderpeptide) of MvdE (SEQ ID 76) (Enzyme-leader-fusions, ELFs) were made as follows. ELFs were made in a three step procedure where, first the enzyme and leader sequence were cloned, second, variants of each were made with N- and C-terminal extensions of 5 (SEQ ID 167), 10 (SEQ ID 170) or 15 (SEQ ID 171) Glycine/Serine, Alanine/Serine, Threonine/Serine amino acid pairs, overlapping PCR was used to join these together to make the constructs leader-G/S(5)-MvdD, leader-G/S(10)-MvdD, leader-G/S(5)-MvdD, and MvdD-G/S(5)-leader, MvdD-G/S(10)-leader, MvdD-G/S(15)-leader as follows.

Specific oligonucleotide primers (Microsynth, Austria) binding to the gene coding for the ATP-grasp ribosomal peptide maturase MvdD (Accno ACC54550) of Planktothrix agardhii NIVA-CYA126/8 (DSMZ GmbH, Germany) were used to amplify the mvdD gene during PCR amplification with Phusion DNA Polymerase (Thermo, Austria) according to the guidelines of the supplier. The resulting amplicon was cloned into pJET1.2 (Thermo, Austria) and resulting recombinant DNA molecules were used for DNA sequencing (Microsynth, Austria) to confirm correct DNA sequences. The resulting plasmid was named MvdD-pJet and stored at −20° C. Specific oligonucleotide primers (Microsynth, Austria) binding to the 5′ beginning of the Microviridin precursor gene mvdE of Planktothrix agardhii NIVA-CYA126/8 were used to amplify the mvdE gene during PCR amplification with Phusion DNA Polymerase (Thermo, Austria) according to the guidelines of the supplier. The resulting amplicon was cloned into pJET1.2 (Thermo, Austria) and resulting recombinant DNA molecules were used for DNA sequencing (Microsynth, Austria) to confirm correct DNA sequences. The resulting plasmid was named LeaderE-pJet and stored at −20° C.

Plasmid Mvd-pJet was used as DNA template for in total six different PCR reactions differing in their individual Primer combinations. A subset of three PCR reactions contained the same reverse primer (EcoRIMvdNelf−) complementary to the Tend of mvdD gene sequence with an EcoRI restriction sequence at its 5′ end. To 3′ each reaction a specific forward primer was added with each of the primers being complementary to the 5′ end of mvdD gene sequence and differing by individual 5″extensions coding for an extra 5 Glycine/Serine, Alanine/Serine, Threonine/Serine (NelfMvdDGS5+) or 10 Glycine/Serine, Alanine/Serine, Threonine/Serine (NelfMvdDGS10+) or 15 Gly-cine/Serine, Alanine/Serine, Threonine/Serine (NelfMvdDGS15+) amino acid pairs. PCR reactions were performed using Phusion DNA polymerase according to the guidelines of the supplier (ThermoFisher, Austria). Resulting PCR products were applied on agarose gelchromatography (Lonza, USA) and stained with PeqGreen dye (Peqlab, Germany) and visualized under UV light (UVP, Germany). Bands representing the correct size were cut out from the gel and used for isolation of the containing DNA amplicons using QiaQuick gel extraction kit (Qiagen, Austria) and stored at −20° C. The second subset of three PCR reactions shared the same forward primer (NdeIMvdCelf+) complementary to the 5′ end of mvdD gene sequence with an NdeI restriction sequence at its 5′ end. To each reaction a specific reverse primer was added with each of the primers being complementary to the 3′ end of mvdD gene sequence and differing by individual 5″extensions coding for an extra 5 Glycine/Serine, Alanine/Serine, Threonine/Serine (CelfMvdDGS5−) or 10 Glycine/Serine, Alanine/Serine, Threonine/Serine (CelfMvdDGS10−) or 15 Glycine/Serine, Alanine/Serine, Threonine/Serine (CelfMvdDGS15−) amino acid pairs. PCR reactions were performed using Phusion DNA polymerase according to the guidelines of the supplier (ThermoFisher, Austria). Resulting PCR products were applied on agarose gelchromatography (Lanza, USA) and stained with PeqGreen dye (Peqlab, Germany) and visualized under UV light (UVP, Germany). Bands representing the correct size were cut out from the gel and used for isolation of the containing DNA amplicons using QiaQuick gel extraction kit (Qiagen, Austria) and stored at −20° C.

Plasmid LeaderE-pJet was used as DNA template for in total six different PCR reactions differing in their individual Primer combinations. A subset of three PCR reactions contained the same forward primer (NdeILeaderENelf+) complementary to the 5′ send of mvdE gene sequence with an NdeI restriction sequence at its 5′ end. To each reaction a specific reverse primer was added with each of the primers being complementary to the 3′ end of mvdE gene sequence and differing by individual 5″extensions coding for an extra 5 Glycine/Serine, Alanine/Serine, Threonine/Serine (NelfLeaderEGS5−) or 10 Glycine/Serine, Alanine/Serine, Threonine/Serine (NelfLeaderEGS10−) or 15 Glycine/Serine, Alanine/Serine, Threonine/Serine (NelfLeaderEGS15−) amino acid pairs. The second subset of three PCR reactions contained the same reverse primer (EcoRILeaderECelf−) complementary to the 3′ end of mvdE gene sequence with an EcoRI restriction sequence at its 5′ end. To each reaction a specific forward primer was added with each of the primers being complementary to the 5′ end of mvdE gene sequence and differing by individual 5″extensions coding for an extra 5 Glycine/Serine, Alanine/Serine, Threonine/Serine (CelfLeaderEGS5+) or 10 Glycine/Serine, Alanine/Serine, Threonine/Serine (CelfLeaderEGS10+) or 15 Glycine/Serine, Alanine/Serine, Threonine/Serine (CelfLeaderEGS15+) amino acid pairs. PCR reactions were performed using Phusion DNA polymerase according to the guidelines of the supplier (ThermoFisher, Austria). Resulting PCR products were applied on agarose gelchromatography (Lonza, USA) and stained with PeqGreen dye (Peqlab, Germany) and visualized under UV light (UVP, Germany). Bands representing the correct size were cut out from the gel and used for isolation of the containing DNA amplicons using QiaQuick gel extraction kit (Qiagen, Austria) and stored at −20° C.

The amplicons from above were used as DNA templates for overlapping PCR reactions. PCR amplicon NelfLeaderEGS5 was used together with amlicon NelfMvdDGS5; NelfLeaderEGS10 together with NelfMvdDGS10 and NelfLeaderEGS15 in combination with NelfMvdDGS15. In all three PCR reactions the forward primer NdeINelfLeader+ and the reverse primer EcoRINelfMvdD− were used and DNA polymerase Q5 (New England Biolabs, Austria) following the instructions of the supplier. Resulting PCR products were applied on agarose gelchromatography (Lonza, USA) and stained with PeqGreen dye (Peglab, Germany) and visualized under UV light (UVP, Germany). Bands representing the correct size were cut out from the gel and used for isolation of the containing DNA amplicons using QiaQuick gel extraction kit (Qiagen, Austria) and cloned into pJET1.2. Resulting recombinant plasmids were used for DNA sequencing to confirm correct DNA sequences. Resulting plasmids were named NelfMvdDGS5, NelfMvdDGS10 and NelfMvdDGS15 indicating N-terminal enzyme leader fusions (Nelf).

The remaining PCR amplicons were used in the following combinations: CelfLeaderEGS5 was used together with CelfMvdDGS5, CelfLeaderEGS10 and CelfMvdDGS10 and amplicons CelfLeaderEGS15 together CelfMvdDGS15. PCR amplification was performed using Q5 DNA polymerase as described above. Resulting PCR products were applied on agarose gelchromatography (Lonza, USA) and stained with PeqGreen dye (Peqlab, Germany) and visualized under UV light (UVP, Germany). Bands representing the correct size were cut out from the gel and used for isolation of the containing DNA amplicons using QiaQuick gel extraction kit (Qiagen, Austria) and cloned into pJET1.2. Resulting recombinant plasmids were used for DNA sequencing to confirm correct DNA sequences. Resulting plasmids were named CelfMvdDGS5, CelfMvdDGS10 and CelfMvdDGS15 indicative for C-terminal enzyme leader fusions. 3 μg of each of the above produced plasmid DNAs were digested with NdeI and EcoRI and resulting fragments were separated by agarose gelchromatography. DNA bands of the expected sizes were isolated using QiaQuick gel extraction kit and cloned into expression plasmid pET28b (Novagen, USA), which was identically digested with NdeI and EcoRI. DNA sequencing (Microsynth, Austria) was used to confirm correct recombinant DNA plasmids named NelfMvdDGS5-pET, NelfMvdDGS10-pET, NelfMvdDGS15-pET and CelfMvdDGS5-pET, CelfMvdDGS10-pET, CelfMvdDGS15-pET.

10 ng of each of the above described plasmids were used to genetically transform electro-competent Escherichia coli BL21 (DE3) (Novagen, USA) using the electroporator Micropulser (BioRad, Austria) with standard setting for E. coli and standard 2 mm width cuvettes (Sigma Aldrich, Austria). Resulting single clones were selected on Kanamycin (Melford Biolaboratories UK) containing LB (Sigma Aldrich) petri dishes (GreinerBioOne, Austria) and one single clone was used to inoculate 11 of Kanamycin containing LB broth. Cultures were incubated in 21 Erlenmayer flasks (Schott, Germany) each containing 500 ml suspension at 37° C. with constant shaking at 220 rpm on a shaker (Innova, Switzerland). Optical density was monitored by spectrophotometrical measurements at 600 nm with a U-2800 spectrophotometer (Hitachi, Austria). After reaching an optical density of 0.6 the suspension was cooled on ice for 5 minutes and supplemented with 0.1 mM IPTG (Isopropyl-β-D-thiogalactopyranosid) (Melford Biolaboratories, UK) and incubated further at 16° C. with shaking at 220 rpm for additional 16 h.

After 16 h cells were sedimented by centrifugation (Sorvall Instruments, USA) for 10 min at 6000 g. The resulting cell paste was frozen at −20° C. in a standard freezer (Liebherr, Austria). Frozen cell paste was weighed and resuspended in 10× (weight per volume) of Lysis buffer. Lysis buffer contained 50 mM Tris (Isopropyl-β-D-thiogalactopyranosid) (Sigma Aldrich, Austria), 300 mM NaCl (Sigma Aldrich, Austria), 5 mM Imidazole (Amresco, USA) pH 8. Cells were disrupted in a M-110P cell disruptor (Microfluidics, USA) until suspension became translucent (one to two cycles). Cell debris and the insoluble protein fraction were separated by centrifugation (Sorvall Instruments, USA) for 30 min at 4° C. with 15.000 g. The supernatant was then transferred into a new centrifugation tube (Thermo Scientific, Austria) and centrifuged again under identical conditions. After second centrifugation the supernatant was mixed with 500 μl Nickel-Agarose resin (Qiagen, Austria) that was previously washed with 2.5 ml (5 vol) of 50 mM Tris pH7.5 (Sigma Aldrich). The resin-supernatant mixture was incubated in a over-head shaker (Stuart Instruments, Austria) at 4° C. for 2 h at 30 rpm. After 2 h the suspension was loaded on a polyprep column (Biorad, Austria) and allowed to drain by gravity force. The resin was washed with 5 ml lysis buffer with 5 mM beta-mercaptoethanol (BME) (Sigma Aldrich, Austria), followed by 3×1 ml wash buffer (50 mM TRIS, 300 mM NaCl, 25 mM imidazole, 5 mM BME pH 8). The protein was eluted with 3×1 ml of elution buffer (50 mM TRIS, 300 mM NaCl, 250 mM Imidazole, 5 mM BME, pH 8).

The elution fractions were combined and desalted using a desalting colum (Econopac, Biorad, Austria) equilibrated with storage buffer (25 mM TRIS, 500 mM NaCl, 10% (w/v) glycerol (Sigma Aldrich, Austria). The protein was aliquoted into 100 μl portions in 0.2 ml tubes (Starlab, Germany) and flash frozen in liquid nitrogen and stored at −80° C. until use.

An aliquot of each of the proteins was checked by SDS-PAGE gel electrophoresis to confirm the purity of the produced ATP-grasp ribosomal peptide maturase MvdD fused to the N-terminal peptide (Leaderpeptide) of the Microviridin precursorpeptide MvdE.

### Example 11: Combined Use of Two Enzyme-Leader-Fusions (Elfs) from Differing Families Acting on the Same Substrate

The above produced Elfs originating from the Prochlorosin and Microviridin families were used together acting on the same substrate. As Elfs are post-translational modifying enzymes that are fused to their cognate leaders it is feasible to combine several of them in order to introduce different ring structures on a shared substrate. Therefore, a substrate peptide was synthesized by solid phase chemistry (JPT, Germany) which comprised of Microviridin and Prochlorosin features. In particular the peptide with the sequence NH2-KPTTVKPPSDFEEWTINVC-COOH (SEQ ID 151) was used as substrate for the Elfs NelfMvdDGS10 in combination with Celf5ProcM.

A reaction mixture of 47 μl composed of 50 mM HEPES at pH7.5, 10 mM MgCl, 0.5 mM TCEP, 2.5 mM ATP, 10 μg peptide and 3 μg of NelfMvdDGS10 was incubated at 37° C. for 6 h. After the incubation time 3 μg of Celf5ProcM was added and incubation time was prolonged for another 12 h at 37° C. After the incubation the reactions were stored at −20° C. 1 μl of the reaction mixture (approx. 400 pmol) was diluted 1:10 with 0.5% formic acid (FA), 0.5% acetonitrile. The dilution was desalted using ZipTip pipette tips (RP-C18, Millipore) according to manufacturer's recommendations for peptide samples. The final eluate was dried in a speedvac and resolubilized in 50 μl 0.5% formic acid (FA), 0.5% acetonitrile. 5 μl of the desalted peptides were analyzed using LC-ESI-mass spectrometry (LTQ FT, Thermo Finnigan). The mass spectra were analyzed according the predicted peptide masses for the unmodified vs. the modified peptide species. The m/z value of the unmodified peptide P2 was 1096.041 (monoisotopical [M+2H-F]2+) as predicted. After enzyme incubation one portion of the used peptide P2 was found showing triple dehydration with a m/z value of 1069.022 (predicted 1069.024 with a deviation of 2 mmu). This proves the loss of three water molecules due to enzyme activities catalyzed by Elf NelfMvdDGS10 and Celf5ProcM.

Results are shown in FIGS. 5-8.

1. A method of biosynthesis of a cyclic peptide by enzymatically transforming a substrate peptide into the cyclic peptide, wherein the substrate peptide is expressed by a displaying genetic package and comprises at least one serine (Ser) or threonine (Thr) residue and at least one cysteine (Cys) residue, and wherein the enzyme is a post-translationally modifying enzyme (PTME) which is a bifunctional thioether bridge-forming dehydratase and cyclase, thereby obtaining the displaying genetic package carrying the cyclic peptide comprising a thioether bridge crosslinking the at least one Ser or Thr to Cys.

2. The method of claim 1, wherein the PTME is from a cyanobacterium from the genus Prochlorococcus or Synechococcus, and comprises:
a) the catalytic sites of a ProcM enzyme selected from the group consisting of ProcM9313 identified by the amino acid sequence SEQ ID NO:1, ProcM9303 identified by the amino acid sequence SEQ ID NO:2, and ProcM9916 identified by the amino acid sequence SEQ ID NO:3; or
b) the amino acid sequence SEQ ID NO:1, SEQ ID NO:2 or SEQ ID NO:3, or an amino acid sequence with at least 60% sequence identity to any of SEQ ID NO:1, SEQ ID NO:2, or SEQ ID NO:3; or
c) a functionally active variant of any of the foregoing.

3. The method of claim 2, wherein:
the functionally active variant of embodiment a) comprises at least one modified catalytic site which is the dehydratase or the cyclase acting region and at least one dehydratase acting region and at least one cyclase acting region, wherein the dehydratase acting region has an amino acid sequence selected from the group consisting of SEQ ID NO:4, SEQ ID NO:6, and SEQ ID NO:8, and wherein the cyclase acting region has an amino acid sequence selected from the group consisting of SEQ ID NO:5, SEQ ID NO:7, and SEQ ID NO:9, and which is characterized by at least one point mutation and at least 60% sequence identity to any of SEQ ID NO:3 to SEQ ID NO:9; or
the functionally active variant of embodiment b) comprises at least one point mutation and/or is a size variant;
and wherein the functionally active variant is capable of dehydrating and cycling amino acid residues of the substrate peptide.

4. The method of claim 1, wherein the PTME is a ProcM enzyme selected from the group consisting of ProcM9313, ProcM9303, and ProcM9916.

5. The method of claim 1, wherein the substrate peptide comprises the sequence Ser/Thr-Xaa(n)-Cys or Cys-Xaa(n)-Ser/Thr, and wherein n=0-100 amino acids.

6. The method of claim 5, wherein the thioether bridge is formed between side-chains of the Ser or Thr to Cys, thereby forming a loop of 2-102 amino acids.

7. (canceled)

8. The method of claim 1, wherein the genetic package is selected from the group consisting of a bacteriophage, a virus, a bacterium, a yeast, and a ribosome, or wherein the genetic package comprises a RNA/DNA-peptide fusion molecule.

9. The method of claim 1, wherein the genetic package is a filamentous phage, wherein the cyclic peptide is immobilised onto the bacteriophage by fusion to a coat protein, and wherein the coat protein is selected from the group consisting of gene III, gene VI, gene VII, gene VIII, and gene IX.

10. The method of claim 1, wherein the cyclic peptide is bound to the surface of the genetic package via a peptide linker and/or a disulfide bridge.

11. The method of claim 1, wherein the substrate peptide is transformed into the cyclic peptide in the soluble form, or upon display by the genetic package.

12. The method of claim 1, wherein a repertoire of variant substrate peptides is transformed in a one-step process, thereby producing a library of cyclic peptides.

13. The method of claim 1, further comprising the step of cleaving the cyclic peptide is from the genetic package.

14. The method of claim 12, wherein the variant substrate peptides comprise randomised peptide sequences to produce the repertoire.

15. (canceled)

16. The method of claim 14, wherein the nucleic acid encoding the substrate peptide is randomised, and wherein the proportion of at least one base at a specified position in the codon of the substrate peptide is varied to bias the codon towards coding for an amino acid selected from the group consisting of serine, threonine, and cysteine.

17-18. (canceled)

19. The method of claim 14, wherein a PTME-leader peptide is ligated to at least one of the substrate peptide or the PTME.

20. (canceled)

21. The method of claim 19, wherein the PTME-leader peptide and/or the PTME are of a Prochlorococcus or Synechococcus origin, or a functionally active derivative thereof.

22. The method of claim 19, wherein at least two different PTME-leader peptides are used which are recognized by one or more PTME.

23. The method of claim 1, wherein at least two different PTMEs are used to transform the substrate peptide into the cyclic peptide, and wherein the PTMEs are from a class selected from the group consisting of a bifunctional dehydratase-cyclase, a dehydratase, a cyclase, a carboxylate-amine ligase, a decarboxylase, a epimerase, a hydroxylase, a peptidase, a dehydratase, a transferase, a esterase, a oxygenase, a isomerase and a transglutaminase.

24. The method of claim 1, wherein the cyclic peptide is a polycyclic peptide comprising at least two heteroatom bridges, and wherein a heteroatom bridge is linking an amino acid side chain to another amino acid residue of the substrate peptide thereby forming a loop.

25. The method of claim 24, wherein the polycyclic peptide comprises overlapping loops and/or loops within loops.

26. A library of immobilised cyclic peptides obtained by the method of claim 1 and comprising at least 106 library members, wherein each peptide has a length of at least 10 amino acids and comprises a thioether bridge forming a loop within the peptide, and wherein the substrate peptides used in the method of claim 1 to obtain the cyclic peptides of the library each further comprise at least one of:
a) a different position of the thioether bridge forming a loop within the substrate peptide; or
b) a different number of loops within the substrate peptide.
